(19) United States
(12) Patent Application Publication
(10) Pub. No.: US 2001/0041673 A1
(43) Pub. Date: Nov. 15, 2001

(54) Combinations of corticotropin releasing factor antagonists and growth hormone secretagogues

(76) Inventor: Anthony A. Fossa, Mystic, CT (US)

Correspondence Address: Gregg C. Benson, Pfizer Inc., Patent Department, MS 4159, Eastern Point Road, Groton, CT, 06340, US

(21) Appl. No.: 09/834,477

(22) Filed: Apr. 13, 2001

Related U.S. Application Data
(60) Provisional application No. 60/196,698, filed on Apr. 13, 2000.

(51) Int. Cl. A61K 38/22; A61K 31/52; A61K 31/517; A61K 31/47

(52) U.S. Cl. 514/21; 514/258; 514/262; 514/261; 514/272; 514/300; 514/310

(57) ABSTRACT

This invention is directed to pharmaceutical compositions comprising corticotropin releasing factor antagonist and growth hormone or growth hormone secretagogues, prodrugs thereof, or pharmaceutically acceptable salts of said compounds or said prodrugs. The invention is also directed to methods of treating heart related diseases (including congestive heart failure) in mammals, and particularly in humans.

### CROSS REFERENCE TO RELATED APPLICATION

This application claims priority of U.S. provisional application Ser. No. 60/196,698, filed Apr. 13, 2000.

### BACKGROUND OF THE INVENTION

This invention relates to pharmaceutical compositions comprising combinations of corticotropin releasing factor (CRF) antagonists and growth hormone or growth hormone secretagogues, prodrugs thereof, and pharmaceutically acceptable salts of said compounds and said prodrugs. These compositions have utility, inter alia, in the treatment of osteoporosis or frailty associated with aging or obesity, in the treatment of cardiovascular or heart related diseases including hypertension, tachycardia, and in particular congestive heart failure, as well as in accelerating bone fracture repair, attenuating protein catabolic response after a major operation, reducing cachexia and protein loss due to chronic illness, accelerating wound healing or accelerating the recovery of burn patients or of patients having undergone major surgery. These utilities are most relevant to mammals, and particularly to humans. Accordingly, this invention also relates to methods of using such compositions for the treatment of the above diseases in mammals, particularly humans.

CRF antagonists are disclosed in U.S. Pat. Nos. 4,605,642 and 5,063,245. Other CRF antagonists are disclosed in International patent publications WO 95/33750; WO 95/34563; WO 94/13661; WO 94/13644; WO 94/13643; WO 94/13676; WO 94/13677; WO 95/33727; WO 98/05661; WO 98/08847; WO 98/08846; and European patent publications EP 778277 and EP 773023. Yet other CRF antagonists are disclosed in the following patent publications: EP 576350; EP 659747; EP 812831; WO 95/10506; WO 96/35689; WO 96/39400; WO 97/00868; WO 97/14684; WO 97/29109; WO 97/29110; WO 97/35539; WO 97/35580; WO 97/35846; WO 97/44038; WO 97/45421; WO 98/03510; WO 98/08821; WO 98/11075; WO 98/15543; WO 98/21200; WO 98/27066; WO 98/29397; WO 98/29413; WO 98/42699; WO 98/35967; WO 98/42706; WO 98/45295; WO 98/47874; WO 98/47903; WO 98/51312; WO 99/01454; WO 99/01439; WO 99/10350; WO 99/12908; WO 99/00373; WO 99/38868; WO 99/51597; WO 99/51599; WO 99/40089; WO 99/51598; and WO 99/51600. Still more CRF antagonists are disclosed in U.S. Pat. Nos. 5,109,111; 5,132,111; 5,245,009; 5,464,847; 5,493,006; 5,510,458; 5,644,057; 5,663,292; 5,668,145; 5,705,646; 5,712,303; and 5,723,608. An overview of the patent literature on CRF antagonists is provided in T. E. Christos and A. Arvanitis, Exp. Opin. Ther. Patents (1998) 8(2):143-152. Many of the above cited publications include information on how to make the CRF antagonists described therein.

The importance of CRF antagonists is set out in the literature, e.g., P. Black, Scientific American: “Science & Medicine,” 1995, 2:16-25; T. Lovenberg, et al., Current Pharmaceutical Design, 1995, 1: 305-316; D. T. Chalmers et al., Trends in Pharmacological Sciences, April 1996, pages 166-172; and U.S. Pat. No. 5,063,245. An outline of the activities possessed by CRF antagonists is found in M. J. Owens et al., 1991, Pharm. Rev., 43:425-473. CRF antagonists are described in the art as being effective in the treatment of stress-related illnesses, mood disorders such as depression, major depressive disorder, single episode depression, recurrent depression, child abuse induced depression, postpartum depression, dysthemia, bipolar disorders, and cyclothymia; chronic fatigue syndrome; eating disorders such as anorexia and bulimia nervosa; generalized anxiety disorder; panic disorder; phobias; obsessive-compulsive disorder; post-traumatic stress disorder; pain perception such as fibromyalgia; headache; gastrointestinal diseases; hemorrhagic stress; ulcers; stress-induced psychotic episodes; fever; diarrhea; post-operative ileus; colonic hypersensitivity; irritable bowel syndrome; Crohn's disease; spastic colon; inflammatory disorders such as rheumatoid arthritis and osteoarthritis; pain; asthma; psoriasis; allergies; osteoporosis; premature birth; hypertension, congestive heart failure; sleep disorders; neurodegenerative diseases such as Alzheimer's disease, senile dementia of the Alzheimer's type, multiinfarct dementia, Parkinson's disease, and Huntington's disease; head trauma; ischemic neuronal damage; excitotoxic neuronal damage; epilepsy; stroke; spinal cord trauma; psychosocial dwarfism; euthyroid sick syndrome; syndrome of inappropriate antidiuretic hormone; obesity; chemical dependencies and addictions; drug and alcohol withdrawal symptoms; infertility; cancer; muscular spasms; urinary incontinence; hypoglycemia and immune dysfunctions including stress induced immune dysfunctions, immune suppression and human immunodeficiency virus infections; and stress-induced infections in humans and animals.

PCT publication WO 97/24369, which is incorporated herein by reference, discloses growth hormone secretagogues of formula III:

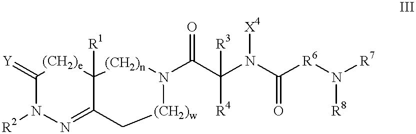

wherein the variables are as defined in WO 97/24369.

PCT publication WO 98/58947, which is incorporated herein by reference, discloses growth hormone secretagogues of formula IV:

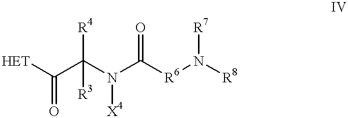

wherein the variables are as defined in WO 98/58947.

Other growth hormones and growth hormone secretagogues that can be used to treat the disorders recited in the methods and compositions of this invention are referred to in PCT international patent application numbers PCT/US97/07516 (published as WO 97/41879) and PCT/DK98/00249 (published as WO 98/58950), as well as in U.S. Pat. Nos. 5,206,235; 5,283,241; and 5,492,916. Many of the above-cited publications disclose how to make or obtain the growth hormone or growth hormone secretagogue described therein. All of the above-cited patent applications and United States patents are incorporated herein by reference in their entirety. Any growth hormone and growth hormone secretagogue, either presently known or yet to be discovered, may be used in the present invention.

### SUMMARY

This invention is directed to pharmaceutical compositions comprising a CRF antagonist, a growth hormone secretagogue or growth hormone, and preferably additionally a pharmaceutically acceptable carrier, vehicle, or diluent.

This invention is also directed to methods for treating or preventing osteoporosis or frailty associated with aging or obesity, cardiovascular or heart related disease, in particular hypertension, tachycardia, and congestive heart failure, accelerating bone fracture repair, attenuating protein catabolic response after a major operation, reducing cachexia and protein loss due to chronic illness, accelerating wound healing, or accelerating the recovery of burn patients or of patients having undergone major surgery, wherein said methods comprise administering to a human or other mammal an amount of a pharmaceutical composition as defined herein, which is effective in treating or preventing the stated disease or condition. This invention is also directed to methods for treating or preventing the diseases or conditions described herein by the co-administration of two separate pharmaceutical compositions. In this latter embodiment, a first composition comprises a CRF antagonist, and a second composition comprises a growth hormone or growth hormone secretagogue. These first and second compositions are preferably co-administered either simultaneously, or in a specifically timed manner.

This invention is also directed to kits comprising a) an amount of a CRF antagonist, in a first unit dosage form; b) an amount of a growth hormone secretagogue or growth hormone in a second unit dosage form; and c) a container.

This invention is also directed to kits comprising a) a pharmaceutical composition comprising an amount of a growth hormone or growth hormone secretagogue, b) a package containing the above composition, and c) a package insert (which may be integral with the package), wherein it is stated on the package insert that the pharmaceutical composition is to be administered simultaneously or in a specifically timed manner with a separate pharmaceutical composition containing at least one CRF antagonist.

This invention is also directed to kits, comprising a) a pharmaceutical composition comprising an amount of a CRF antagonist, b) a package containing the above composition, and c) a package insert that may be integral with the package, wherein it is stated on the package insert that the pharmaceutical composition is to be administered simultaneously or in a specifically timed manner with a pharmaceutical composition containing at least one growth hormone or growth hormone secretagogue.

A group of preferred CRF antagonists for use in the compositions, methods, and kits of the present invention are those wherein the CRF antagonist is a compound of formula:

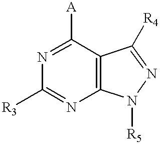

or a pharmaceutically acceptable acid addition salt thereof, wherein A is NR₁R₂, CR₁R₂R₁₁, or C(═CR₁R₁₂)R₂, NHCR₁R₂R₁₁, OCR₁R₂R₁₁, SCR₁R₂R₁₁, NHNR₁R₂, CR₂R₁₁NHR₁, CR₂R₁₁OR₁, CR₂R₁₁SR₁ or C(O)R₂;

R₁ is hydrogen, or C₁-C₆ alkyl which may be substituted by one or two substituents R₆ independently selected from the group consisting of hydroxy, fluoro, chloro, bromo, iodo, C₁-C₆ alkoxy, O—C(O)—(C₁-C₆ alkyl), O—C(O)—N(C₁-C₄ alkyl)(C₁-C₂ alkyl); amino, NH(C_{-C}₄ alkyl), S(C₁-C₆ alkyl), OC(O)NH(C₁-C₄ alkyl), N(C₁-C₂alkyl)C(O)(C₁-C₄ alkyl), NHC(O)(C₁-C₄ alkyl), COOH, CO(C₁-C₄ alkyl), C(O)NH(C₁-C₄alkyl), C(O)N(C₁-C₄ alkyl)(C₁-C₂ alkyl), SH, CN, NO₂, SO(C₁-C₄ alkyl); SO₂(C₁-C₄alkyl), SO₂NH(C₁-C₄ alkyl), SO₂N(C₁-C₄ alkyl)(C₁-C₂ alkyl), and said C₁-C₆ alkyl may have one or two double or triple bonds;

R₂ is C₁-C₁₂ alkyl, aryl or (C₁-C₁₀ alkylene)aryl wherein said aryl is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, azaindolyl, oxazolyl, or benzoxazolyl; 3- to 8-membered cycloalkyl or (C₁-C₆ alkylene) cycloalkyl, wherein said cycloalkyl may have one or two of O, S or N—Z, wherein Z is hydrogen, substituted, independently, for one or two carbons of said cycloalkyl, C₁-C₄ alkyl, benzyl or C₁-C₄ alkanoyl, wherein R² may be substituted independently by from one to three of chloro, fluoro, or C₁-C₄ alkyl, or one of hydroxy, bromo, iodo, C₁-C₆ alkoxy, OC(O)(C₁-C₆ alkyl), O—C—N(C₁-C₄ alkyl)(C₁-C₂ alkyl), S(C₁-C₆ alkyl), NH₂, NH(C₁-C₂ alkyl), N(C₁-C₄ alkyl) C(O)(C₁-C₄ alkyl), NHC(O)(C₁-C₄ alkyl), COOH, C(O)O(C₁-C₄ alkyl), C(O)NH(C₁-C₄ alkyl), C(O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂alkyl), and wherein said C₁-C₁₂ alkyl or C₁-C₁₀ alkylene may have one to three double or triple bonds; or

NR₁R₂ or CR₁R₂R₁₁ may form a 4- to 8-membered ring optionally having one or two double bonds or one or two of O, S or N—Z wherein Z is hydrogen, C₁-C₄ alkyl, benzyl, or C₁-C₄ alkanoyl;

R₃ is hydrogen, C₁-C₆ alkyl, fluoro, chloro, bromo, iodo, hydroxy, amino, O(C₁-C₆ alkyl), NH(C₁-C₆ alkyl), N(C₁-C₄ alkyl)(C₁-C₂ alkyl), SH, S(C₁-C₄ alkyl), SO(C₁-C₄alkyl), or SO₂(C₁-C₄ alkyl), wherein said C₁-C₄ alkyl and C₁-C₆ alkyl may have one or two double or triple bonds and may be substituted by from 1 to 3 R₇ substituents independently selected from the group consisting of hydroxy, amino, C₁-C₃ alkoxy, dimethylamino, diethylamino, methylamino, ethylamino, NHC(O)CH₃, fluoro, chloro or C₁-C₃ thioalkyl;

R₄ is hydrogen, C₁-C₆ alkyl, fluoro, chloro, bromo, iodo, C₁-C₆ alkoxy, amino, NH(C_{-C}₆ alkyl), N(C₁-C₆ alkyl) (C₁-C₂ alkyl), SOₙ(C₁-C₆ alkyl), wherein n is 0, 1 or 2, cyano, hydroxy, carboxy, or amido, wherein said C₁-C₆ alkyls may be substituted by one to three of hydroxy, amino, carboxy, amido, NHC(O)(C₁-C₄ alkyl), NH(C₁-C₄alkyl), N(C₁-C₄alkyl)(C₁-C₂alkyl), C(O)O(C₁-C₄ alkyl), C₁-C₃ alkoxy, C₁-C₃thioalkyl, fluoro, bromo, chloro, iodo, cyano or nitro;

R₅ is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzoisothiazolyl, thiazolyl, isoxazolyl, benzisoxazolyl, benzimidazolyl, triazolyl, pyrazolyl, pyrrolyl, indolyl, pyrrolopyridyl benzoxazolyl, oxazolyl, pyrrolidinyl, thiazolidinyl, piperazinyl, piperidinyl, or tetrazolyl, wherein each one of the above groups may be substituted independently by from one to three of fluoro, chloro, bromo, formyl, C₁-C₆ alkyl, C₁-C₆ alkoxy or trifluoromethyl, or one of hydroxy, iodo, cyano, nitro, amino, cyclopropyl, NH(C₁-C₄ alkyl), N(C₁-C₄ alkyl)(C₁-C₂ alkyl), COO(C₁-C₄ alkyl), CO(C₁-C₄ alkyl), SO₂NH(C₁-C₄ alkyl), SO₂N(C₁-C₄ alkyl)(C₁-C₂alkyl), SO₂NH₂, NHSO₂(C₁-C₄ alkyl), S(C₁-C₆ alkyl), SO₂(C₁-C₆ alkyl), wherein said C₁-C₄ alkyl and C₁-C₆ alkyl may have one double or triple bond and may be substituted by one or two of fluoro, chloro, hydroxy, amino, methylamino, dimethylamino or acetyl; with the proviso that R₅ is not unsubstituted phenyl;

R₁₁ is hydrogen, hydroxy, fluoro, chloro, COO(C₁-C₂ alkyl), cyano, or CO(C₁-C₂ alkyl); and

R₁₂ is hydrogen or C₁-C₄ alkyl;

with the provisos that:

(a) A is not straight chain C₁-C₁₂ alkyl;

(b) when R₃ is hydrogen, A is benzyl or phenethyl, and R₄ is fluoro, chloro, bromo or iodo, then R₅ is not 5′-deoxy-ribofuranosyl or 5′-amino-5′-deoxy-ribofuranosyl; and

(c) when R⁵ is phenyl, said phenyl is substituted by two or three substituents.

Another group of preferred CRF antagonists for use in the compositions, methods, and kits of the present invention are those wherein the CRF antagonist is a compound of formula:

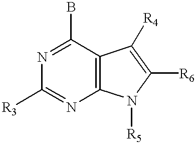

or a pharmaceutically acceptable acid addition salt thereof, wherein B is NR₁R₂, CR₁R₂R₁₁, C(═CR₂R₁₂)R₁, NHR₁R₂R₁₁, OCR₁R₂R₁₁, SCR₁R₂R₁₁, NHNR₁R₂, CR₂R₁₁NHR₁, CR₂R₁₁OR₁, CR₂R₁₁SR₁, or C(O)R₂;

R₁ is hydrogen, or C₁-C₆ alkyl which may be substituted by one or two substituents R₇ independently selected from the group consisting of hydroxy, fluoro, chloro, bromo, iodo, C₁-C₈ alkoxy, O—C(⊚O)—(C₁-C₆ alkyl), O—C(═O)NH(C₁-C₄ alkyl), O—C(═O)—N(C₁-C₄ alkyl)(C₁-C₂ alkyl), amino, NH(C₁-C₄ alkyl), N(C₁-C₂ alkyl)(C₁C₄ alkyl), S(C₁-C₆ alkyl), N(C₁-C₄ alkyl)C(═O)(C₁-C₄ alkyl), NH(C₁-C₄ alkyl), COOH, C(═O)O(C₁-C₄ alkyl), C(═O)NH(C₁-C₄ alkyl), C(═O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂alkyl), and said C₁-C₆ alkyl may contain one or two double or triple bonds;

R₂ is C₁-C₁₂ alkyl, aryl or (C₁-C₁₀ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, pyrrolopyridyl, oxazolyl, or benzoxazolyl; 3- to 8-membered cycloalkyl or (C₁-C₆alkylene) cycloalkyl, wherein said cycloalkyl may contain one or two of O, S or N—Z wherein Z is hydrogen, C₁-C₄ alkyl, benzyl or C₁-C₄ alkanoyl, wherein R₂ may be substituted independently by from one to three of chloro, fluoro, or C₁-C₄ alkyl, or one of hydroxy, bromo, iodo, C₁-C₆ alkoxy, O—C(═O)—(C₁-C₆ alkyl), O—C—N(C₁-C₄ alkyl)(C₁-C₂ alkyl), S(C₁-C₆ alkyl), NH₂, NH(C₁-C₂ alkyl), N(C₁-C₂ alkyl) (C₁-C₄alkyl), N(C₁-C₄)—C(═O)(C₁-C₄ alkyl), NHC(═O)(C₁-C₄), COOH, C(═O)O(C₁-C₄ alkyl), C(═O)NH(C₁-C₄alkyl), C(═O)N(C₁-C₄ alkyl)(C₁-C₂ alkyl), SH, CN, NO₂, SO(C₁-C₄ alkyl), SO₂(C₁-C₄alkyl), SO₂NH(C₁-C₄ alkyl), SO₂N(C₁-C₄ alkyl)(C₁-C₂ alkyl), and wherein said C₁-C₁₂alkyl or C₁-C₁₀ alkylene may contain one to three double or triple bonds; or

NR₁R₂ or CR₁R₂R₁₁ may form a saturated 3- to 8 membered carbocyclic ring of which the 5- to 8-membered ring contain one or two double bonds or one or two of O, S or N—Z wherein Z is hydrogen, C₁-C₄ alkyl, benzyl or C₁-C₄ alkanoyl;

R₃ is hydrogen, C₁-C₆ alkyl, fluoro, chloro, bromo, iodo, hydroxy, amino, O(C₁-C₆ alkyl), NH(C₁-C₆ alkyl), N(C₁-C₄ alkyl)(C₁-C₂ alkyl), SH, S(C₁-C₄ alkyl), SO(C₁-C₄-alkyl), or SO₂(C₁-C₄ alkyl), wherein said C₁-C₄ alkyl and C₁-C₆ alkyl may contain from one or two double or triple bonds and may be substituted by from 1 to 3 substituents R₈ independently selected from the group consisting of hydroxy, amino, C₁-C₃ alkoxy, dimethylamino, diethylamino, methylamino, ethylamino, NHCH₃, fluoro, chloro or C₁-C₃ thioalkyl;

R₄ and R₆ are each independently hydrogen, C₁-C₆ alkyl, fluoro, chloro, bromo, iodo, C₁-C₆ alkoxy, amino, NH(C₁-C₆ alkyl), N(C₁-C₆ alkyl)(C₁-C₂ alkyl), SOₙ(C₁-C₆ alkyl), wherein n is 0, 1 or 2, cyano, hydroxy, carboxy, or amido, wherein said C₁-C₆ alkyls may be substituted by one to three of hydroxy, amino, carboxy, amido, NHC(═O)(C₁-C₄ alkyl), NH(C₁-C₄ alkyl), N(C₁-C₄ alkyl)(C₁-C₂ alkyl), C(═O)O(C₁-C₄ alkyl), C₁-C₃ alkoxy, C₁-C₃ thioalkyl, fluoro, bromo, chloro, iodo, cyano or nitro;

R₅ is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, azaindolyl, benzoxazolyl, oxazolyl, pyrrolidinyl, thiazolidinyl, morpholinyl, piperidinyl, piperazinyl, tetrazolyl, or 3- to 8-membered cycloalkyl or 9- to 12-membered bicycloalkyl, optionally containing one to three of O, S or N—Z wherein Z is hydrogen, C₁-C₄ alkyl, C₁-C₄ alkanoyl, phenyl or phenylmethyl, wherein each one of the above groups may be substituted independently by from one to four of fluoro, chloro, C₁-C₆ alkyl, C₁-C₆ alkoxy or trifluoromethyl, or one of bromo, iodo, cyano, nitro, amino, NH(C₁-C₄ alkyl), N(C₁-C₄)(C₁-C₂ alkyl), COO(C₁-C₄ alkyl), CO(C₁ -C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), SO₂(C₁-C₆ alkyl), wherein said C₁-C₄ alkyl and C₁-C₆ alkyl may be substituted by one or two of fluoro, chloro, hydroxy, amino, methylamino, dimethylamino or acetyl; with the proviso that R₅ is not unsubstituted phenyl;

R₁₁ is hydrogen, hydroxy, fluoro, chloro, COO(C₁-C₂ alkyl), cyano, or CO(C₁-C₂ alkyl); and

R₁₂ is hydrogen or C₁-C₄ alkyl; with the proviso that (1) when R₅ is 4-bromophenyl, R₃ is hydrogen, and R₄ and R₆ are methyl, then B is not methylamino or ethyl, and (2) when R₅ is 4-bromophenyl, and R₃, R₄ and R₆ are methyl, then B is not 2-hydroxyethylamino.

Another group of preferred CRF antagonists for use in the compositions, methods, and kits of the present invention are those wherein the CRF antagonist is a compound of formula:

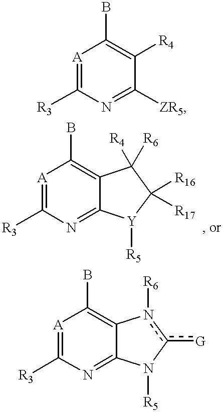

or a pharmaceutically acceptable acid addition salt thereof, wherein

A is CR₇or N;

B is NR₁R₂, CR₁R₂R₁₁, C(═CR₂R₁₂)R₁, NHCHR₁R₂, OCHR₁R₂, SCHR₁R₂, CHR₂OR₁₂, CHR₂SR₁₂, C(S)R₂ or C(O)R₂;

G is oxygen, sulfur, NH, NH₃, hydrogen, methoxy, ethoxy, trifluoromethoxy, methyl, ethyl, thiomethoxy, NH₂, NHCH₃, N(CH₃)₂ or trifluromethyl;

Y is CH or N;

Z is NH, O, S, N (C₁-C₂ alkyl), or CR₁₃R₁₄, wherein R₁₃ and R₁₄ are each independently hydrogen, trifluoromethyl, or C₁-C₄ alkyl, or one of R₁₃ and R₁₄ may be cyano, chloro, bromo, iodo, fluoro, hydroxy, O(C₁-C₂ alkyl), amino, NH(C₁-C₂ alkyl), or CR₁₃R₁₄ may be C═O or cyclopropyl;

R₁ is C₁-C₆ alkyl which may be substituted by one or two substituents R₈ independently selected from the group consisting of hydroxy, fluoro, chloro, bromo, iodo, C₁-C₄ alkoxy, O—CO—(C₁-C₄ alkyl), O—CO—NH(C₁-C₄ alkyl), O—CO—N(C₁-C₄ alkyl)(C₁-C₂ alkyl), NH(C₁-C₄ alkyl), N(C₁-C₂ alkyl)(C_{-C}₄ alkyl), S(C₁-C₄ alkyl), N(C₁-C₄ alkyl)CO(C₁-C₄ alkyl), NHCO(C₁-C₄ alkyl), COO(C₁-C₄ alkyl), CONH(C₁-C₄ alkyl), CON(C₁-C₄ alkyl)(C₁-C₂ alkyl), S(C₁-C₄ alkyl), CN, NO₂, SO(C₁-C₄ alkyl), SO₂(C₁-C₄ alkyl), and said C₁-C₆ alkyl or C₁-C₄ alkyl may contain one double or triple bond;

R₂ is C₁-C₁₂ alkyl, aryl or (C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, or benzoxazolyl; 3- to 8-membered cycloalkyl or (C₁-C₆ alkylene)cycloalkyl, wherein said cycloalkyl may contain one or two of O, S or N-R₉ wherein R₉ is hydrogen, or C₁-C₄ alkyl, wherein the above defined R₂ may be substituted independently by from one to three of chloro, fluoro, or C₁-C₄ alkyl, or one of bromo, iodo, C₁-C₆ alkoxy, O—CO—(C₁-C₆ alkyl), O—CO—N(C₁-C₄ alkyl)(C₁-C₂ alkyl), S(C₁-C₆ alkyl), CN, NO₂, SO(C₁-C₄ alkyl), or SO₂(C₁-C₄ alkyl), and wherein said C₁-C₁₂ alkyl or C₁-C₄ alkylene may contain one double or triple bond; or

NR₁R₂ or CR₁R₂R₁₁ may form a saturated 5- to 8-membered carbocyclic ring which may contain one or two double bonds or one or two of O or S;

R₃ is methyl, ethyl, fluoro, chloro, bromo, iodo, cyano, methoxy, OCF₃, methylthio, methylsulfonyl, CH₂OH or CH₂OCH₃;

R₄ is hydrogen, C₁-C₄ alkyl, fluoro, chloro, bromo, iodo, C₁-C₄ alkoxy, amino, nitro, NH(C₁-C₄ alkyl), N(C₁-C₄ alkyl)(C₁-C₂ alkyl), SOₙ(C₁-C₄ alkyl), wherein n is 0, 1 or 2, cyano, hydroxy, CO(C₁-C₄ alkyl), CHO, or COO(C₁-C₄ alkyl), wherein said C₁-C₄ alkyl may contain one or two double or triple bonds and may be substituted by one or two of hydroxy, amino, carboxy, NHCOCH₃, NH(C₁-C₂ alkyl), N(C₁-C₂ alkyl)₂, COO(C₁-C₄ alkyl), CO(C₁-C₄ alkyl), C₁-C₃ alkoxy, C₁-C₃ thioalkyl, fluoro, chloro, cyano or nitro;

R₅ is phenyl, naphthyl, thienyl, benzothienyl, pyridyl, quinolyl, pyrazinyl, pyrimidyl, furanyl, benzofuranyl, benzothiazolyl, or indolyl, wherein each one of the above groups R₅ is substituted independently by from one to three of fluoro, chloro, C₁-C₆ alkyl, or C₁-C₆ alkoxy, or one of hydroxy, iodo, bromo, formyl, cyano, nitro, trifluoromethyl, amino, NH(C₁-C₄ alkyl), N(C₁-C₆)(C₁-C₂ alkyl), COOH, COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), or SO₂(C₁-C₆ alkyl), wherein said C₁-C₄ alkyl and C₁-C₆ alkyl may be substituted by one or two of fluoro, hydroxy, amino, methylamino, dimethylamino or acetyl;

R₆ is hydrogen, or C₁-C₆ alkyl, wherein said C₁-C₆ alkyl may be substituted by one hydroxy, methoxy, ethoxy or fluoro;

R₇ is hydrogen, C₁-C₄ alkyl, fluoro, chloro, bromo, iodo, cyano, hydroxy, O(C₁-C₄ alkyl), C(O)(C₁-C₄ alkyl), or C(O)O(C₁-C₄ alkyl), wherein the C₁-C₄ alkyl groups may be substituted with one hydroxy, chloro or bromo, or one to three fluoro;

R₁₁ is hydrogen, hydroxy, fluoro, or methoxy;

R₁₂ is hydrogen or C₁-C₄ alkyl; and

R₁₆ and R₁₇ are each independently hydrogen, hydroxy, methyl, ethyl, methoxy, or ethoxy, except that they are not both methoxy or ethoxy, and CR₄R₆ and CR₁₆R₁₇ each independently may be C═O.

Another group of preferred CRF antagonists for use in the compositions, methods, and kits of the present invention are those wherein the CRF antagonist is a compound of formula:

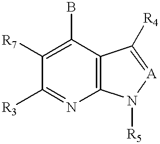

or a pharmaceutically acceptable acid addition salt thereof, wherein

A is N or —CR₆;

B is —NR₁R₂, —CR₁R₂R₁₁, —C(═CR₂R₁₂)R₁, —NHCHR₁R₂, —OCHR₁R₂, —SCHR₁R₂, —CHR₂OR₁₂, —CHR₂SR₁₂, —C(S)R₁ or —C(O)R₁;

R₁ is C₁-C₆ alkyl which may optionally be substituted with one or two substituents independently selected from the group consisting of hydroxy, fluoro, chloro, bromo, iodo, C₁-C₄ alkoxy, —O—CO—(C₁-C₄ alkyl), —O—CO—NH(C₁-C₄ alkyl), —O—CO—N(C₁-C₄ alkyl)(C₁-C₂ alkyl), —NH(C₁-C₄ alkyl), —N(C₁-C₂ alkyl)(C₁-C₄ alkyl), —S(C₁-C₄ alkyl), —N(C₁-C₄ alkyl)CO(C₁-C₄ alkyl), —NHCO(C₁-C₄ alkyl), —COO(C₁-C₄ alkyl), —CONH(C₁-C₄ alkyl), —CON(C₁-C₄ alkyl)(C₁-C₂ alkyl), CN, NO₂, —SO(C₁-C₄ alkyl) —SO₂(C₁-C₄ alkyl), and wherein any of the foregoing C₁-C₄ alkyl and C₁-C₆ alkyl groups may optionally contain one carbon-carbon double or triple bond;

R₂ is C₁-C₁₂ alkyl, aryl, —(C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, oxazolyl, or benzoxazolyl; or 3- to 8- membered cycloalkyl or —(C₁-C₆ alkylene)cycloalkyl, wherein one or two of the ring carbons of said cycloalkyl having at least 4 ring members and the cycloalkyl moiety of said —(C₁-C₆ alkylene)cycloalkyl having at least 4 ring members may optionally be replaced by an oxygen or sulfur atom or by N—Z wherein Z is hydrogen; or C₁-C₄ alkyl, and wherein each of said groups R₂ may optionally be substituted with from one to three substituents independently selected from chloro, fluoro, and C₁-C₄ alkyl, or by one substituent selected from bromo, iodo, C₁-C₆ alkoxy, —O—CO—(C₁-C₆ alkyl), —S(C₁-C₆ alkyl), —COO(C₁-C₄ alkyl), CN, NO₂, —SO(C₁-C₄ alkyl), and —SO₂(C₁-C₄ alkyl), and wherein said C₁-C₁₂ alkyl and the C₁-C₄ alkylene moiety of said -(C₁-C₄ alkylene)aryl may optionally contain one carbon-carbon double or triple bond;

or —NR₁R₂ may form a saturated 5- to 8-membered heterocyclic ring, or —CHR₁R₂ may form a saturated 5- to 8-membered carbocyclic ring, wherein each of these rings may optionally contain one or two carbon-carbon double bonds and wherein one or two of the carbon atoms of each of these rings may optionally be replaced with a sulfur or oxygen atom;

R₃ is C₁-C₄ alkyl, fluoro, chloro, bromo, iodo, —CH₂OH, —CH₂OCH₃, —O(C₁-C₃ alkyl), —S(C₁-C₃ alkyl), or —SO₂(C₁-C₃ alkyl), wherein said C₁-C₃ alkyl may optionally contain one carbon-carbon double or triple bond;

R₄ is hydrogen, C₁-C₆ alkyl, fluoro, chloro, bromo, iodo, C₁-C₄ alkoxy, amino, —NHCH₃, —N(CH₃)₂, —CH₂OH, —CH₂OCH₃, or —SOₙ(C₁-C₄ alkyl), wherein n is 0, 1 or 2, cyano, hydroxy, —CO(C₁-C₄ alkyl), —CHO, or —COO(C₁-C₄ alkyl) wherein the C₁-C₄ alkyl moieties in the foregoing R₄ groups may optionally contain one carbon-carbon double or triple bond;

R₅ is phenyl, naphthyl, thienyl, benzothienyl, pyridyl, pyrimidyl, benzofuranyl, pyrazinyl or benzothiazolyl, wherein each one of said groups R₅ may optionally be substituted with from one to three substituents independently selected from fluoro, chloro, C₁-C₆ alkyl and C₁-C₆ alkoxy, or by one substituent selected from iodo, hydroxy, bromo, formyl, cyano, nitro, amino, trifluoromethyl, —NH(C₁-C₄ alkyl), —N(C₁-C₆)(C₁-C₂ alkyl), —COO(C₁-C₄ alkyl), —CO(C₁-C₄ alkyl), —COOH, —SO₂NH(C₁-C₄ alkyl), —SO₂N(C₁-C₄ alkyl)(C₁-C₂ alkyl), —SO₂NH₂, —NHSO₂(C₁-C₄ alkyl), —S(C₁-C₆ alkyl) and —SO₂(C₁-C₆ alkyl), wherein each of said C₁-C₄ alkyl and C₁-C₆ alkyl moieties in the foregoing R⁵ groups may optionally be substituted with one to three fluorine atoms;

R₆ is hydrogen, C₁-C₄ alkyl, fluoro, chloro, bromo, iodo, —CH₂OH, —CH₂OCH₃, or C₁-C₄ alkoxy;

R₇ is hydrogen, C₁-C₄ alkyl, fluoro, chloro, bromo, iodo, —O(C₁-C₄ alkyl), cyano, —CH₂OH, —CH₂O(C₁-C₂ alkyl), —CO(C₁-C₂ alkyl), or —COO(C₁-C₂ alkyl);

R₁₁ is hydrogen, hydroxy, fluoro, or methoxy; and

R₁₂ is hydrogen or C₁-C₄ alkyl;

with the proviso that when A is N, then: (a) B is not unsubstituted alkyl; (b) R₅ is not unsubstituted phenyl or monosubstituted phenyl; and (c) R₃ is not unsubstituted alkyl.

Another group of preferred CRF antagonists for use in the compositions, methods, and kits of the present invention are those wherein the CRF antagonist is a compound of formula:

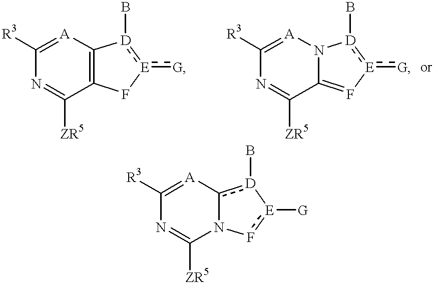

or a pharmaceutically acceptable salt thereof, wherein

the dashed lines represent optional double bonds;

A is nitrogen or CR⁷;

B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR₂R₁₀OR¹, —CR²R¹⁰SR¹ or —COR²;

D is nitrogen and is single bonded to all atoms to which it is attached, or D is carbon and is either double bonded to E in formulas I and II or double bonded to the adjacent carbon atom common to both fused rings in formula II, or D is CH and is single bonded to E in formulas I and II;

E is nitrogen, CH or carbon;

F is oxygen, sulfur, CHR⁴ or NR⁴ when it is single bonded to E and F is nitrogen or CR⁴ when it is double bonded to E;

G, when single bonded to E, is hydrogen, C₁-C₄ alkyl, —S(C₁-C₄ alkyl), —O(C₁-C₄ alkyl), NH₂, —NH(C₁-C₄ alkyl) or —N(C₁-C₂ alkyl)(C₁-C₄ alkyl), wherein each of the C₁-C₄ alkyl groups of G may optionally be substituted with one hydroxy, —O(C₁-C₂ alkyl) or fluoro group; G, when double bonded to E, is oxygen, sulfur or NH; and G, when E is nitrogen and double bonded to D or F, is absent;

R¹ is hydrogen, C₁-C₆ alkyl optionally substituted with one or two substituents R⁸ independently selected from hydroxy, fluoro, chloro, bromo, iodo, C₁-C₄ alkoxy, CF₃, —C(═O)O—(C₁-C₄)alkyl, —OC(═O)(C₁-C₄ alkyl), —OC(═O)N(C₁-C₄ alkyl)(C₁-C₂alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;

R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, —OC(═O)(C₁-C₆ alkyl), —OC(═O)N(C₁-C₄ alkyl)(C₁-C₂ alky), —S(C₁-C₆ alkyl), amino, —NH(C₁-C₂ alkyl), —N(C₁-C₂ alkyl)(C₁-C₄ alkyl), —N(C₁-C₄ alkyl)—CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);

—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;

R³ is hydrogen, C₁-C₄ alkyl, —O(C₁-C₄ alkyl), chloro, fluoro, bromo, iodo, —CN, —S(C₁-C₄ alkyl) or —SO₂(C₁-C₄ alkyl) wherein each of the (C₁-C₄ alkyl) moieties in the foregoing R³ groups may optionally be substituted with one substituent R⁹ selected from hydroxy, fluoro and (C₁-C₂ alkoxy);

each R⁴ is, independently, hydrogen, (C₁-C₆ alkyl), fluoro, chloro, bromo, iodo, hydroxy, cyano, amino, nitro, —O(C₁-C₄ alkyl), —N(C₁-C₄ alkyl)(C₁-C₂ alkyl), —S(C₁-C₄ alkyl), —SO(C₁-C₄ alkyl), —SO₂(C₁-C₄)alkyl, —CO(C₁-C₄ alkyl), —C(═O)H or —C(═O)O(C₁-C₄ alkyl), wherein each of the (C₁-C₆ alkyl) and (C₁-C₄ alkyl) moieties in the foregoing R⁴ groups may optionally contain one or two double or triple bonds and may optionally be substituted with one or two substituents independently selected from hydroxy, amino, C₁-C₃ alkoxy, dimethylamino, methylamino, ethylamino, —NHC(═O)CH₃, fluoro, chloro, C₁-C₃ thioalkyl, —CN, —COOH, —C(═O)O(C₁-C₄ alkyl), —C(═O)(C₁-C₄ alkyl) and —NO₂;

R⁵ is phenyl, naphthyl, thienyl, benzothienyl, pyridyl, quinolyl, pyrazinyl, furanyl, benzofuranyl, benzothiazolyl, benzisothiazolyl, benzisoxazolyl, benzimidazolyl, indolyl, benzoxazolyl or C₃-C₈ cycloalkyl wherein one or two of the carbon atoms of said cycloalkyl rings that contain at least 5 ring members may optionally and independently be replaced by an oxygen or sulfur atom or by NZ⁴ wherein Z⁴ is hydrogen, C₁-C₄ alkyl or benzyl; and wherein each of the foregoing R⁵ groups is substituted with from one to four substituents R¹² wherein one to three of said substituents may be selected, independently, from chloro, C₁-C₆ alkyl and —O(C₁-C₆ alkyl) and one of said substituents may be selected from bromo, iodo, formyl, —CN, —CF₃, —NO₂, —NH₂, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;

R⁷ is hydrogen, C₁-C₄ alkyl, halo, cyano, hydroxy, —O(C₁-C₄ alkyl) —C(═O)(C₁-C₄ alkyl), —C(═O)O(C₁-C₄ alkyl), —OCF₃, —CF₃, —CH₂OH, —CH₂O (C₁-C₄ alkyl);

R¹⁰ is hydrogen, hydroxy, methoxy or fluoro;

R¹¹ is hydrogen or C₁-C₄ alkyl; and

Z is NH, oxygen, sulfur, —N(C₁-C₄ alkyl), —NC(═O)(C₁-C₂ alkyl), NC(═O)O(C₁-C₂ alkyl) or CR¹³R¹⁴ wherein R¹³ and R¹⁴ are independently selected from hydrogen, trifluoromethyl and methyl with the exception that one of R¹³ and R¹⁴can be cyano;

with the proviso that: (a) in the five membered rings of structures I, II and III, there can not be two double bonds adjacent to each other; and (b) when R⁴ is attached to nitrogen, it is not halo, cyano or nitro.

Another group of preferred CRF antagonists for use in the compositions, methods, and kits of the present invention are those wherein the CRF antagonist is a compound of formula:

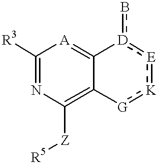

wherein the dashed lines represent optional double bonds; or a pharmaceutically acceptable salt thereof, wherein

A is nitrogen or CR⁷;

B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR², and is single bonded to D; or B is —CR¹R², and is double bonded to D and D is carbon;

D is nitrogen or CR⁴ and is single bonded to all atoms to which it is attached, or D is carbon and is double bonded to E or double bonded to B;

E is oxygen, nitrogen, sulfur, C═O, C═S, CR⁶R¹², NR₆ or CR⁶; or E is a two atom spacer, wherein one of the atoms is oxygen, nitrogen, sulfur, C═O, C═S, CR⁶R¹², NR⁶ or CR⁶, and the other is CR⁶R¹² or CR⁹;

K and G are each, independently, C═O, C═S, sulfur, oxygen, CHR⁸ or NR⁸ when single bonded to both adjacent ring atoms, or nitrogen or CR⁸ when it is double bonded to an adjacent ring atom;

the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;

R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄ alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;

R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);

—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ³ wherein Z³ is hydrogen or C₁-C₄ alkyl;

R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);

R⁴ is hydrogen, C₁-C₂ alkyl, hydroxy or fluoro;

each R⁶, R⁸ and R⁹ that is attached to a carbon atom is selected, independently, from hydrogen, C₁-C₂ alkyl, fluoro, chloro, bromo, iodo, hydroxy, hydroxymethyl, formyl, trifluoromethyl, cyano, amino, nitro, —O(C₁-C₂ alkyl), —N(C₁-C₂ alkyl)(C₁-C₂ alkyl), —S(C₁-C₂ alkyl), —CO(C₁-C₂ alkyl), —C(═O)H or —C(═O)O(C₁-C₂alkyl), wherein each of the C₁-C₂ alkyl moieties in the foregoing R⁶, R⁸, and R⁹ groups may optionally contain one double or triple bond; and each R⁶, R⁸, and R⁹ that is attached to a nitrogen atom is selected, independently, from hydrogen and C₁-C₄ alkyl;

R⁵ is substituted phenyl, naphthyl, pyridyl or pyrimidyl, wherein each of the foregoing R⁵ groups is substituted with from two to four substituents R¹⁵, wherein from one to three of said substituents may be selected, independently, from chloro, C₁-C₆ alkyl, —O(C₁-C₆ alkyl) and —(C₁-C₆ alkylene)O(C₁-C₆ alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;

R⁷ is hydrogen, methyl, halo, hydroxy, methoxy, —C(═O)(C₁-C₂ alkyl), —C(═O)O(C₁-C₂ alkyl), trifluoromethoxy, hydroxymethyl, trifluoromethyl or formyl;

R¹⁰ is hydrogen, hydroxy, methoxy or fluoro;

R¹¹ is hydrogen or C₁-C₄ alkyl;

R₁₂ is hydrogen or methyl; and

Z is NH, oxygen, sulfur, —N(C₁-C₄ alkyl), or CR¹³R¹⁴ wherein R¹³ and R¹⁴ are independently selected from hydrogen, and methyl with the exception that one of R¹³ and R¹⁴ may optionally be cyano;

with the proviso that: (a) in the six or seven membered rings of structures in formula I, there can not be two double bonds adjacent to each other; and (b) when D is carbon and is double bonded to B, then B is CR¹R².

Another group of preferred CRF antagonists for use in the compositions, methods, and kits of the present invention are those wherein the CRF antagonist is a compound of formula:

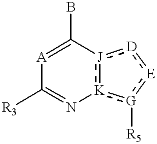

or a pharmaceutically acceptable salt thereof, wherein

the dashed lines represent optional double bonds;

A is nitrogen or CR⁷;

B is —NR¹R², —CR¹R²R¹⁰—C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR²;

J and K are each independently nitrogen or carbon and both J and K are not nitrogens;

D and E are each selected, independently, from nitrogen, CR⁴, C═O, C═S, sulfur, oxygen, CR⁴R⁶ and NR⁸;

G is nitrogen or carbon;

the ring containing D, E, G, K, and J in formula I may be a saturated or unsaturated 5-membered ring and may optionally contain one or two double bonds and may optionally contain from one to three heteroatoms in the ring and may optionally have one or two C═O or C═S groups;

R¹ is C₁-C₆ alkyl optionally substituted with one or two substituents independently selected from hydroxy, fluoro, chloro, bromo, iodo, —O—(C₁-C₄ alkyl), CF₃, —C(═O)O—(C₁-C₄ alkyl), —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;

R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);

—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;

R³ is hydrogen, C₁-C₄ alkyl, —O(C₁-C₄ alkyl), chloro, fluoro, bromo, iodo, (C₁-C₂ alkylene)-O—(C₁-C₂ alkyl), (C₁-C₂ alkylene)-OH, or —S(C₁-C₄ alkyl);

each R⁴ is, independently, hydrogen, (C₁-C₆ alkyl), fluoro, chloro, bromo, iodo, hydroxy, cyano, amino, (C₁-C₂ alkylene)-OH, CF₃, CH₂SCH₃, nitro, —O(C₁-C₄ alkyl), —N(C₁-C₄ alkyl)(C₁-C₂ alkyl), —S(C₁-C₄ alkyl), —CO(C₁-C₄ alkyl), —C(═O)H or —C(═O)O(C₁-C₄ alkyl);

R⁶ is hydrogen, methyl or ethyl;

R⁸ is hydrogen or C₁-C₄ alkyl;

R⁵ is phenyl, pyridyl, pyrazinyl, pyrimidyl, pyridazinyl and wherein each of the foregoing R⁵ groups is substituted with from one to four substituents R¹³ wherein one to three of said substituents may be selected, independently, from fluoro, chloro, C₁-C₆ alkyl and —O(C₁-C₆ alkyl) and one of said substituents may be selected from bromo, iodo, formyl, OH, (C₁-C₄ alkylene)-OH, (C₁-C₄ alkylene)-O—(C₁-C₂ alkyl), —CN, —CF₃, —NO₂, —NH₂, —NH(C₁-C₄ alkyl), —N(C₁-C₂ alkyl)(C₁-C₆ alkyl), —OCO(C₁-C₄ alkyl), (C₁-C₄ alkylene)-O—(C₁-C₄ alkyl), —S(C₁-C₆ alkyl), (C₁-C₄ alkylene)-S—(C₁-C₄ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally have one or two double bonds;

R⁷ is hydrogen, C₁-C₄ alkyl, halo (e.g., chloro, fluoro, iodo or bromo), hydroxy, —O(C₁-C₄ alkyl), —C(═O)(C₁-C₄ alkyl), —C(═O)O(C₁-C₄ alkyl), —OCF₃, —CF₃, —CH₂OH or —CH₂O(C₁-C₂ alkyl);

R¹⁰ is hydrogen, hydroxy, methoxy or fluoro;

R¹¹ is hydrogen or C₁-C₄ alkyl; and with the proviso that: a) when both J and K are carbons and D is CR⁴ and E is nitrogen, then G can not be nitrogen; (b) when both J and K are carbons and D and G are nitrogens, then E can not be CR⁴ or C═O or C═S; (c) when both J and K are carbons and D and E are carbons, then G can not be nitrogen; (d) when G is carbon, it must be double banded to E; and (e) in the ring containing J, K, D, E and G, there can not be two double bonds adjacent to each other.

Another group of preferred CRF antagonists for use in the compositions, methods, and kits of the present invention are those wherein the CRF antagonist is a compound of formula:

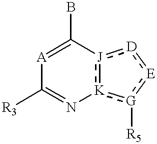

or a pharmaceutically acceptable salt thereof, wherein

the dashed lines represent optional double bonds;

A is nitrogen or CR⁷;

B is —NR¹R² —CR¹R²R¹⁰—C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR²;

J and K are each independently nitrogen or carbon and both J and K are not nitrogens;

D and E are each selected, independently, from nitrogen, CR⁴, C═O, C═S, sulfur, oxygen, CR⁴R⁶ and NR⁸;

G is nitrogen or carbon;

the ring containing D, E, G, K, and J in formula I may be a saturated or unsaturated 5-membered ring and may optionally contain one or two double bonds and may optionally contain from one to three heteroatoms in the ring and may optionally have one or two C═O or C═S groups;

R¹ is C₁-C₆ alkyl optionally substituted with one or two substituents independently selected from hydroxy, fluoro, chloro, bromo, iodo, —O—(C₁-C₄ alkyl), CF₃, —C(═O)O—(C₁-C₄ alkyl), —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;

R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);

—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;

R³ is hydrogen, C₁-C₄ alkyl, —O(C₁-C₄ alkyl), chloro, fluoro, bromo, iodo, (C₁-C₂ alkylene)-O—(C₁-C₂ alkyl), (C₁-C₂ alkylene)-OH, or —S(C₁-C₄ alkyl);

each R⁴ is, independently, hydrogen, (C₁-C₆ alkyl), fluoro, chloro, bromo, iodo, hydroxy, cyano, amino, (C₁-C₂ alkylene)-OH, CF₃, CH₂SCH₃, nitro, —O(C₁-C₄ alkyl), —N(C₁-C₄ alkyl)(C₁-C₂ alkyl), —S(C₁-C₄ alkyl), —CO(C₁-C₄ alkyl), —C(═O)H or —C(═O)O(C₁-C₄ alkyl);

R⁶ is hydrogen, methyl or ethyl;

R⁸ is hydrogen or C₁-C₄ alkyl;

R⁵ is phenyl, pyridyl, pyrazinyl, pyrimidyl, pyridazinyl and wherein each of the foregoing R⁵ groups is substituted with from one to four substituents R¹³ wherein one to three of said substituents may be selected, independently, from fluoro, chloro, C₁-C₆ alkyl and —O(C₁-C₆ alkyl) and one of said substituents may be selected from bromo, iodo, formyl, OH, (C₁-C₄ alkylene)-OH, (C₁-C₄ alkylene)-O—(C₁-C₂ alkyl), —CN, —CF₃, —NO₂, —NH₂, —NH(C₁-C₄ alkyl), —N(C₁-C₂ alkyl)(C₁-C₆ alkyl), —OCO(C₁-C₄ alkyl), (C₁-C₄ alkylene)-O—(C₁-C₄ alkyl)-S—(C₁-C₆ alkyl), (C₁-C₄ alkylene)—S—(C₁-C₄ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally have one or two double bonds;

R⁷ is hydrogen, C₁-C₄ alkyl, halo (e.g., chloro, fluoro, iodo or bromo), hydroxy, —O(C₁-C₄ alkyl), —C(═O)(C₁-C₄ alkyl), —C(═O)O(C₁-C₄ alkyl), —OCF₃, —CF₃, —CH₂OH or —CH₂O(C₁-C₂ alkyl);

R¹⁰ is hydrogen, hydroxy, methoxy or fluoro;

R¹¹ is hydrogen or C₁-C₄ alkyl; and

with the proviso that: a) when both J and K are carbons and D is CR⁴ and E is nitrogen, then G can not be nitrogen; (b) when both J and K are carbons and D and G are nitrogens, then E can not be CR⁴ or C═O or C═S; (c) when both J and K are carbons and D and E are carbons, then G can not be nitrogen; (d) when G is carbon, it must be double banded to E; and (e) in the ring containing J, K, D, E and G, there can not be two double bonds adjacent to each other.

Another group of preferred CRF antagonists for use in the compositions, methods, and kits of the present invention are those wherein the CRF antagonist is a compound of formula:

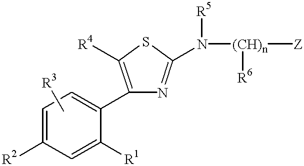

wherein each of R¹ and R² is independently a halogen atom; a C₁-C₅ hydroxyalkyl radical; C₁-C₅ alkyl; C₇-C₁₀ aralkyl; C₁-C₅ alkoxy; trifluoromethyl; nitro; nitrile; a group —SR where R is hydrogen, a C₁-C₅ alkyl radical or a C₇-C₁₀ aralkyl radical; a group S—CO—R where R is a C₁-C₅ alkyl radical or aralkyl in which the aryl portion is C₆-C₈ and the alkyl portion is C₁-C₄; a group —COOR′ where R′ is hydrogen or C₁-C₅ alkyl; a group —CONR′R″ where R′ and R″ are as defined above for R′; a group —NR′R″ where R′ and R″ are as previously defined for R′; a group —CONRaRb or NRaRb, where Ra and Rb, taken together with the nitrogen atom to which they are attached, form a 5- to 7-membered heterocyclic ring; or a group —NHCO-NR′R″, where R′ and R″ are as defined above for R′; R³ is hydrogen or as defined for R¹ and R² is a hydrogen atom; C₁₋₅ alkyl; halogen; a hydroxymethyl group; or a formyl group; R⁵ is C₁-C₅ alkyl; a C₃-C₇ cycloalkyl group; a cycloalkylalkyl group in which the cycloalkyl portion is C₃-C₇ and the alkyl portion is C₁-C₅; or C₅-C₆ alkenyl; n is 0 or 1; R⁶ is C₁₋₅ alkyl; alkoxyal in which the alkyl portions are C₁-C₅; C₃-C₇ cycloalkyl; a cycloalkylalkyl group in which the cycloalkyl portion is C₃-C₇ and the alkyl portion is C₁-C₅; a cycloalkyloxyalkyl radical in which the cycloalkyl is C₃-C₇ and the alkyl is C₁-C₄; a hydroxyalkyloxyalkyl radical in which the alkyls are C₂-C₁₀; or an alkoxyalkyloxyalkyl radical in which the alkyls are C₃-C₁₂; and Z is an optionally substituted bi- or tricyclic aromatic or heteroaromatic group; or a stereoisomer or addition salt thereof.

Another group of preferred CRF antagonists for use in the compositions, methods, and kits of the present invention are those wherein the CRF antagonist is a compound of formula:

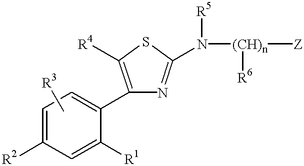

wherein each of R¹ and R² is independently a halogen atom; a C₁-C₅ hydroxyalkyl radical; C₁-C₅ alkyl; C₇-C₁₀ aralkyl; C₁-C₅ alkoxy; trifluoromethyl; nitro; nitrile; a group —SR where R is hydrogen, a C₁-C₅ alkyl radical or a C₇-C₁₀ aralkyl radical; a group S—CO—R where R is a C₁-C₅ alkyl radical or aralkyl in which the aryl portion is C₆-C₈ and the alkyl portion is C₁-C₄; a group —COOR′ where R′ is hydrogen or C₁-C₅ alkyl; a group —CONR′R″ where R′ and R″ are as defined above for R′; a group —NR′R″ where R′ and R″ are as previously defined for R′; a group —CONRaRb or NRaRb, where Ra and Rb, taken together with the nitrogen atom to which they are attached, form a 5- to 7-membered heterocyclic ring; or a group —NHCO-NR′R″, where R′ and R″ are as defined above for R′; R³ is hydrogen or as defined for R¹ and R² is a hydrogen atom; C₁₋₅ alkyl; halogen; a hydroxymethyl group; or a formyl group; R⁵ is C₁-C₅ alkyl; a C₃-C₇ cycloalkyl group; a cycloalkylalkyl group in which the cycloalkyl portion is C₃-C₇ and the alkyl portion is C₁-C₅; or C₅-C₆ alkenyl; n is 0 or 1; R⁶ is C₁₋₅ alkyl; alkoxyalk in which the alkyl portions are C₁-C₅; C₃-C₇ cycloalkyl; a cycloalkylalkyl group in which the cycloalkyl portion is C₃-C₇ and the alkyl portion is C₁-C₅; a cycloalkyloxyalkyl radical in which the cycloalkyl is C₃-C₇ and the alkyl is C₁-C₄; a hydroxyalkyloxyalkyl radical in which the alkyls are C₂-C₁₀; or an alkoxyalkyloxyalkyl radical in which the alkyls are C₃-C₁₂; and Z is an optionally substituted bi- or tricyclic aromatic or heteroaromatic group; or a stereoisomer or addition salt thereof.

Another group of preferred CRF antagonists for use in the compositions, methods, and kits of the present invention are those wherein the CRF antagonist is a compound of formula:

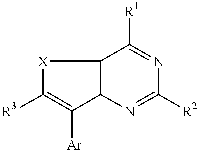

or a stereoisomer or pharmaceutically acceptable acid addition salt form thereof, wherein

X is S, SO or SO₂;

R¹ is NR⁴R⁵ or OR⁵;

R² is C₁-C₆alkyl, C₁-C₆alkyloxy or C₁-C₆alkylthio;

R³ is hydrogen, C₁-C₆ alkyl, C₁-C₆ alkylsulfonyl, C₁-C₆ alkylsulfoxy or C₁-C₆alkylthio;

R⁴ is hydrogen, C₁₋₆alkyl, mono- or di(C₃-C₆cycloalkyl)methyl, C₃-C₆cycloalkyl, C₃-C₆alkenyl, hydroxyC₁-C₆alkyl, C₁-C₆alkylcarbonyloxyC₁-C₆alkyl or C₁-C₆alkyloxyC₁-C₆alkyl;

R⁵ is C₁-C₈alkyl, mono- or di(C₃-C₆Cycloalkyl)methyl, Ar¹CH₂, C₃-C₆alkenyl, C₁-C₆alkyloxyC₁-C₆alkyl, hydroxyC₁-C₆ alkyl, thienylmethyl, furanylmethyl, C₁-C₆alkylthioC₁-C₆ alkyl, morpholinyl, mono- or di(C₁-C₆alkyl)aminoC₁-C₆alkyl, di(C₁-C₆alkyl)amino, C₁-C₆alkylcarbonyC₁-C₆alkyl, C₁-C₆ alkyl substituted with imidazolyl; or a radical of formula-Alk-O—CO—Ar I;

or R⁴ and R⁵ taken together with the nitrogen atom to which they are attached may form a pyrrolidinyl, piperidinyl, homopiperidinyl or morpholinyl group, optionally substituted with C₁-C₆alkyl or C₁-C₆alkyloxyC₁-C₆ alkyl;

Ar is phenyl; phenyl substituted with 1, 2 or 3 substituents independently selected from halo, C₁-C₆alkyl, trifluoromethyl, hydroxy, cyano, C₁-C₆alkyloxy, benzyloxy, C₁-C₆ alkylthio, nitro, amino and mono- or di(C₁-C₆alkyl)amino; pyridinyl; pyridinyl substituted with 1, 2 or 3 substituents independently selected from halo, C₁-C₆alkyl, trifluoromethyl, hydroxy, cyano, C₁-C₆ alkyloxy, benzyloxy, C₁-C₆alkylthio, nitro, amino, mono- or di(C₁-C₆alkyl)amino and piperidinyl; and wherein said substituted phenyl may optionally be further substituted with one or more halogens;

Ar¹ is phenyl; phenyl substituted with 1, 2 or 3 substituents each independently selected from halo, C₁-C₆ alkyl, C₁-C₆alkyloxy, di(C₁-C₆alkyl)aminoC₁-C₆alkyl trifluoromethyl, and C₁-C₆alkyl substituted with morpholinyl; or pyridinyl; and Alk is C₁-C₆ alkanediyl.

Another group of preferred CRF antagonists for use in the compositions, methods, and kits of the present invention are those wherein the CRF antagonist is a compound selected from the group consisting of:

4-(1-ethyl-propoxy)-3,6-dimethyl-2-(2,4,6-trimethylphenoxy)-pyridine;

butyl-[2,5-dimethyl-7-(2,4,6-trimethylphenyl)-6,7-dihydro-5H-pyrrolo[2,3-d]pyrimidin-4-yl]-ethyl-amine;

4-(butyl-ethylamino)-2,5-dimethyl-7-(2,4,6-trimethylphenyl)-5,7-dihydro-pyrrolo[2,3-d]pyrimidin-6-one;

4-(1-ethylpropoxy)-2,5-dimethyl-6-(2,4,6-trimethylphenoxy)-pyrimidine;

N-butyl-N-ethyl-2,5-dimethyl-NN-(2,4,6trimethylphenyl)-pyrimidine-4,6-diamine;

[4-(1-ethyl-propoxy)-3,6-dimethyl-pyridin-2-yl]-(2,4,6-trimethylphenyl)-amine;

6-(ethyl-propyl-amino)-2,7-dimethyl-9-(2,4,6-trimethylphenyl)-7,9-dihydro-purin-8-one;

3-{(4-methyl-benzyl)-[3,6-dimethyl-1-(2,4,6-trimethylphenyl)-1H-pyrazolo[3,4d]pyrimidin-4-yl]-amino}-propan-1-ol;

diethyl-[6-methyl-3-methylsulfanyl-1-(2,4,6-trichlorophenyl)-1H-pyrazolo[3,4-d]pyrimidin-4-yl]-amine;

2-{butyl-[6-methyl-3-methylsulfanyl-1-(2,4,6-trichlorophenyl)-1H-pyrazolo[3,4-d]pyrimidin-4-yl]-amino}-ethanol;

dibutyl-[6-methyl-3-methylsulfanyl-1-(2,4,6-trichlorophenyl)-1H-pyrazolo[3,4-d]pyrimidin-4-yl}-amine;

butyl-ethyl-[6-methyl-3-methylsulfanyl-1-(2,4,6-trichlorophenyl)-1H-pyrazolo[3,4-d]pyrimidin-4-yl]-amine;

butyl-ethyl-[6-methyl-3-methylsulfonyl-1-(2,4,6-trichlorophenyl)-1H-pyrazolo[3,4-d]pyrimidin-4-yl]-amine;

butyl-cyclopropylmethyl-[6-methyl-3-methylsulfanyl-1-(2,4,6-trichlorophenyl)-1H-pyrazolo[3,4-d]pyrimidin-4-yl]-amine;

di-1-propyl-[6-methyl-3-methylsulfanyl-1-(2,4,6-trichlorophenyl)-1H-pyrazolo[3,4-d]pyrimidin-4-yl]-amine;

diallyl-[6-methyl-3-methylsulfanyl-1-(2,4,6-trichlorophenyl)-1H-pyrazolo[3,4-d]pyrimidin-4-yl]-amine;

butyl-ethyl-[6-chloro-3-methylsulfanyl-1-(2,4,6-trichlorophenyl)-1H-pyrazolo[3,4-d]pyrimidin-4-yl]-amine;

butyl-ethyl-[6-methoxy-3-methylsulfanyl-1-(2,4,6-trichlorophenyl)-1H-pyrazolo[3,4-d]pyrimidin-4-yl]-amine;

propyl-ethyl-[3,6-dimethyl-1-(2,4,6-trimethylphenyl)-1H-pyrazolo[3,4-d]pyrimidin-4-yl]-amine;

4-(1-ethyl-propyl)-6-methyl-3-methylsulfanyl-1-(2,4,6-trimethylphenyl)-1H-pyrazolo[3,4-d]pyrimidine;

n-butyl-ethyl-[2,5-dimethyl-7-(2,4,6-trimethylphenyl)-7H-pyrrolo[2,3-d]pyrimidin-4-yl]amine;

di-n-propyl-[2,5-dimethyl-7-(2,4,6-trimethylphenyl)-7H-pyrrolo[2,3-d]pyrimidin-4-yl]amine;

ethyl-n-propyl-[2,5-dimethyl-7-(2,4,6-trimethylphenyl)-7H-pyrrolo[2,3-d]pyrimidin-4-yl]amine;

diethyl-2,5-dimethyl-7-(2,4,6-trimethylphenyl)-7H-pyrrolo[2,3-d]pyrimidin-4-yl]amine;

n-butyl-ethyl-[2,5,6-trimethyl-7-(2,4,6-trimethylphenyl)-7H-pyrrolo[2,3-d]pyrimidin-4-yl]amine;

2-{N-n-butyl-N-[2,5-dimethyl-7-(2,4,6-trimethylphenyl)-7H-pyrrolo[2,3-d]pyrimidin4-yl]amino}-ethanol;

4-(1-ethyl-propyl)-2,5,6-trimethyl-7-(2,4,6-trimethylphenyl)-7H-pyrrolo[2,3-d]pyrimidine;

n-butyl-ethyl-[2,5-dimethyl-7-(2,4-dimethylphenyl)-7H-pyrrolo[2,3-d]pyrimidin-4-yl]amine;

2,5-dimethyl-7-(2,4,6-trimethylphenyl)-7H-pyrrolo[2,3-d]pyrimidyl-4-y]-(1-ethyl-propyl)amine;

butyl-[3,6-dimethyl-1-(2,4,6-trimethylphenyl)-1H-pyrazolo[3,4-b]pyridin-4-yl]-ethylamine;

[3,6-dimethyl-1-(2,4,6-trimethylphenyl)-1H-pyrazolo[3,4,b]pyridin-4-yl]-(1-methoxymethylpropyl)-amine;

4-(1-methoxymethylpropoxy)-3,6-dimethyl-1-(2,4,6-trimethylphenyl)-1H-pyrazolo[3,4-b]pyridine;

(1-ethylpropyl)-[3,5,6-trimethyl-1-(2,4,6-trimethylphenyl)-1H-pyrazolo[3,4-b]pyridin-4-yl]-amine;

4-(1-ethylpropoxy)-2,5-dimethyl-7-(2,4,6-trimethylphenyl)-7H-pyrrolo[2,3-b]pyridine;

4-(1-ethylpropoxy)-2,5,6-trimethyl-7-(2,4,6-trimethylphenyl)-7H-pyrrolo[2,3-b]pyridine;

4-(1-ethylpropoxy)-2,5-dimethyl-7-(2,6-dimethyl-4-bromophenyl)-7H-pyrrolo[2,3-b]pyridine;

2,5,6-trimethyl-7-(1-propylbutyl)-4-(2,4,6-trimethylphenoxy)-7H-pyrrolo[2,3-d]pyrimidine;

1-(1-ethylpropyl)-6-methyl-4-(2,4,6-trimethylphenylamino)-1,3-dihydro-imidazo[4,5-c]pyridin-2-one;

9-(1-ethylpropyl)-2-methyl-6-(2,4,6-trimethylphenylamino)-7,9-dihydro-purin-8-one;

1-(1-ethylpropyl)-6-methyl-4-(2,4,6-trimethylphenoxy)-1,3-dihydro-imidazo[4,5-c]pyridin-2-one;

1-(1-ethylpropyl)-6-methyl-4-(2,4,6-trimethylphenoxy)-1H-imidazo[4,5-c]pyridine;

1(1-ethylpropyl)-3,6-dimethyl-4-(2,4,6-trimethylphenoxy)-1,3-dihydro-imidazo[4,5-c]pyridin-2-one;

1-(1-ethylpropyl)-3,6-dimethyl-4-(2,4,6-trimethylphenylamino)-1,3-dihydro-imidazo[4,5-c]pyridin-2-one;

1-(1-ethyl-propyl)-4,7-dimethyl-5-(2,4,6-trimethyl-phenoxy)-1,4-dihydro-2H-pyrido[3,4-b]pyrazin-3-one;

1-(1-ethyl-propyl)-4,7-dimethyl-5-(2,4,6-trimethyl-phenoxy)-1,2,3,4-tetrahydro-pyrido[3,4-b]pyrazine;

1-(1-ethyl-propyl)-7-methyl-5-(2,4,6-trimethyl-phenoxy)-1,2,3,4-tetrahydro-pyrido[3,4-b]pyrazine;

1-(1-ethyl-propyl)-7-methyl-2-oxo-5-(2,4,6-trimethyl-phenoxy)-1,2,3,4-tetra-hydro-[1,6]naphthyridine-3-carboxylic acid methyl ester;

1-(1-ethyl-propyl)-7-methyl-2-oxo-5-(2,4,6-trimethyl-phenoxy)-1,2,3,4-tetra-hydro-[1,6]naphthyridine-3-carboxylic acid isopropyl ester;

1-(1-ethyl-propyl)-7-methyl-5-(2,4,6-trimethyl-phenoxy)-3,4-dihydro-1H-[1,6]naphthyridin-2-one;

1-(1-ethyl-propyl)-7-methyl-5-(2,4,6-trimethyl-phenoxy)-1,2,3,4-tetrahydro-[1,6]naphthyridine;

1-(1-ethyl-propyl)-7-methyl-5-(2,4,6-trimethyl-phenoxy)-1,4-dihydro-2H-3-oxa-1,6-diaza-naphthalene;

1-(1-ethyl-propyl)-4,7-dimethyl-5-(2,4,6-trimethyl-phenoxy)-1,4-dihydro-2H-3-oxa-1,6-diaza-naphthalene;

1-(1-ethyl-propyl)-3,7-dimethyl-5-(2,4,6-trimethyl-phenoxy)-3,4-dihydro-1H-3-oxa-[1,6]-naphthyridin-2-one;

1-(1-ethyl-propyl)-3,3,6-trimethyl-4-(2,4,6-trimethyl-phenoxy)-2,3-dihydro-1H-pyrrolo[3,2-c]pyridine;

7-(1-ethyl-propoxy)-5-methyl-3-(2,4,6-trimethyl-phenyl)-pyrazolo[1,5-a]pyrimidine;

[2,5-dimethyl-3-(2,4,6-trimethyl-phenyl)-pyrazolo[1,5-a]pyrimidin-7-yl]-(1-ethyl-propyl)-amine;

(1-ethyl-propyl)-[5-methyl-3-(2,4,6-trimethyl-phenyl)-pyrazolo[1,5-a]pyrimidin-7-yl]-amine;

7-(1-ethyl-propoxy)-2,5-dimethyl-3-(2,4,6-trimethyl-phenyl)-pyrazolo[1,5-a]pyrimidine;

[2,5-dimethyl-3-(2,4,6-trimethyl-phenyl)-pyrazolo[1,5-a]pyrimidin-7-yl]-ethyl-propyl-amine;

[6-bromo-5-bromomethyl-3-(2,4,6-trimethyl-phenyl)-3H-[1,2,3]triazolo[4,5-b]pyridin-7-yl]-(1-ethyl-propyl)-amine;

(1-ethyl-propyl)-[5-methyl-3-(2,4,6-trimethyl-phenyl)-3H-[1,2,3]triazolo[4,5-b]pyridin-7-yl]-amine;

[6-bromo-5-methyl-3-(2,4,6-trimethyl-phenyl)-3H-[1,2,3]triazolo[4,5-b]pyridin-7-yl]-(1-ethyl-propyl)-methyl-amine;

7-(1-ethyl-propoxy)-5-methyl-3-(2,4,6-trimethyl-phenyl)-3H-[1,2,3]triazolo[4,5-b]pyridine;

4-(1-ethyl-propoxy)-2,5-dimethyl-7-(2,4,6-trimethyl-phenyl)-5H-pyrrolo[3,2-d]pyrimidine;

(±)-2,5-dimethyl-4-(tetrahydro-furan-3-yloxy)-7-(2,4,6-trimethyl-phenyl)-5H-pyrrolo-[3,2-d]pyrimidine;

2,5-dimethyl-4-(S)-(tetrahydro-furan-3-yloxy)-7-(2,4,6-trimethyl-phenyl)-5H-pyrrolo-[3,2-d]pyrimidine;

2,5-dimethyl-4-(1-propyl-butoxy)-7-(2,4,6-trimethyl-phenyl)-5H-pyrrolo[3,2-d]pyrimidine;

4-sec-butylsulfanyl-2,5-dimethyl-7-(2,4,6-trimethyl-phenyl)-5H-pyrrolo[3,2-d]pyrimidine;

4-(butyl-ethyl-amino)-2,6-dimethyl-8-(2,4,6-trimethyl-phenyl)-5,8-dihydro-6H-pyrido[2,3-d]pyrimidin-7-one;

8-(1-ethyl-propoxy)-6-methyl-4-(2,4,6-trimethyl-phenyl)-3,4-dihydro-1H-pyrido[2,3-b]pyrazin-2-one;

8-(1-ethyl-propoxy)-6-methyl-4-(2,4,6-trimethyl-phenyl)-1,2,3,4-tetrahydro-pyrido[2,3-b]pyrazine;

4-(1-ethyl-propoxy)-2-methyl-8-(2,4,6-trimethyl-phenyl)-quinoline;

5-(1-ethyl-propoxy)-7-methyl-1-(2,4,6-trimethyl-phenyl)-1,4-dihydro-2H-3-oxa-1,8-diaza-naphthalene;

5-(1-ethyl-propoxy)-7-methyl-1-(2,4,6-trimethyl-phenyl)-1,2-dihydro-3-oxa-1,8-diaza-naphthalen-4-one;

8-(1-ethyl-propoxy)-1,6-dimethyl-4-(2,4,6-trimethyl-phenyl)-1,2,3,4-tetrahydro-pyrido[2,3-b]pyrazine;

(1-ethyl-propyl)-[2-methyl-8-(2,4,6-trimethyl-phenyl)-quinolin-4-yl]-amine;

4-(butyl-ethyl-amino)-2,6-dimethyl-8-(2,6-dimethyl-4-bromo-phenyl)-5,8-dihydro-6H-pyrido[2,3-d]pyrimidin-7-one;

4-(butyl-ethyl-amino)-2-methyl-8-(2,6-dimethyl-4-bromo-phenyl)-5,8-dihydro-6H-pyrido[2,3-d]pyrimidin-7-one;

4-(1-ethyl-propoxy)-2-m ethyl-8-(2,6-dimethyl-4-bromo-phenyl)-5,8-dihydro-6H-pyrido[2,3-d]pyrimidin-7-one;

(butyl-ethyl)-[2-methyl-8-(2,6-dimethyl-4-bromo-phenyl)-5,6,7,8-tetrahydro-pyrido[2,3-d]pyrimidin-4-yl]-amine;

(propy1-ethyl)-[2-methyl-8-(2,6-dimethyl-4-bromo-phenyl)-5,6,7,8-tetrahydro-pyrido[2,3-d]pyrimidin-4-yl]-amine;

(diethyl)-[2-methyl-8-(2, 6-dimethyl-4-bromo-phenyl )-5,6,7,8-tetrahydro-pyrido[2,3-d]pyrimidin-4-yl]-amine;

(1-ethyl-propyl)-[2-methyl-8-(2,6-dimethyl-4-bromo-phenyl)-5,6,7,8-tetrahydro-pyrido[2,3-d]pyrimidin-4-yl]-amine;

(1-ethyl-propoxy)-2-methyl-8-(2,6-dimethyl-4-bromo-phenyl)-5,6,7,8-tetrahydro-pyrido[2,3-d]pyrimidine;

4-(butyl-ethyl-amino)-2-methyl-8-(2,4,6-trimethyl-phenyl)-5,8-dihydro-6H-pyrido[2,3-d]pyrimidin-7-one;

4-(1-ethyl-propoxy)-2-methyl-8-(2,4,6-trimethyl-phenyl)-5,8-dihydro-6H-pyrido[2,3-d]pyrimidin-7-one;

(butyl-ethyl)-[2-methyl-8-(2,4,6-trimethyl-phenyl)-5,6,7,8-tetrahydro-pyrido[2,3-d]pyrimidin-4-yl]-amine;

(propyl-ethyl)-[2-methyl-8-(2,4,6-trimethyl-phenyl)-5,6,7,8-tetrahydro-pyrido-[2,3-d]pyrimidin-4-yl]-amine;

(diethyl)-[2-methyl-8-(2,4,6-trimethyl-phenyl)-5,6,7,8-tetrahydro-pyrido[2,3-d]pyrimidin-4-yl]-amine;

(1-ethyl-propyl)-[2-methyl-8-(2,4,6-trimethyl-phenyl)-5,6,7,8,tetrahydro-pyrido[2,3-d]pyrimidin-4-yl]-amine;

(1-ethyl-propoxy)-2-methyl-8-(2,4,6-trimethyl-phenyl)-5,6,7,8,tetrahydro-pyrido[2,3-d]pyrimidine;

8-(1-ethyl-propoxy)-6-methyl-4-(2,6-dimethyl-4-bromo-phenyl)-3,4-dihydro-1H-pyrido[2,3-b]pyrazin-2-one;

8-(1-ethyl-propoxy)-6-methyl-4-(2,6-dimethyl-4-bromo-phenyl)-1,2,3,4-tetrahydro- pyrido[2,3-b]pyrazine;

4-(1-ethyl-propoxy)-2-methyl-8-(2,6-dimethyl-4-bromo-phenyl)-quinoline;

5-(1-ethyl-propoxy)-7-methyl-1-(2,6-dimethyl-4-bromo-phenyl)-1,4-dihydro-2H-3-oxa-1,8-diaza-naphthalene;

5-(1-ethyl-propoxy)-7-methyl-1-(2,6-dimethyl-4-bromo-phenyl)-1,2-dihydro-3-oxa-1,8-diaza-naphthalen-4-one;

8-(1-ethyl-propoxy)-1,6-dimethyl-4-(2,6-dimethyl-4-bromo-phenyl)-1,2,3,4-tetrahydro-pyrido[2,3-b]pyrazine;

(1-ethyl-propyl)-[2-methyl-8-(2,6-dimethyl-4-bromo-phenyl)-quinolin-4-yl]-amine;

4-(butyl-ethyl-amino)-2,6-dimethyl-8-(2,6-dimethyl-4-chloro-phenyl)-5,8-dihydro-6H-pyrido[2,3-d]pyrimidin-7-one;

8-(1-ethyl-propoxy)-6-methyl-4-(2,6-dimethyl-4-chloro-phenyl)-3,4-dihydro-1H-pyrido[2,3-b]pyrazin-2-one;

8-(1-ethyl-propoxy)-6-methyl-4-(2,6-dimethyl-4-chloro-phenyl)-1,2,3,4-tetrahydro- pyrido[2,3-b]pyrazine;

4-(1-ethyl-propoxy)-2-methyl-8-(2,6-dimethyl-4-chloro-phenyl)-quinoline;

5-(1-ethyl-propoxy)-7-methyl-1-(2,6-dimethyl-4-chloro-phenyl)-1,4-dihydro-2H-3-oxa-1,8-diaza-naphthalene;

5-(1-ethyl-propoxy)-7-methyl-1-(2,6-dimethyl-4-chloro-phenyl)-1,2-dihydro-3-oxa-1,8-diaza-naphthalen-4-one;

8-(1-ethyl-propoxy)-1,6-dimethyl-4-(2,6-dimethyl-4-chloro-phenyl)-1,2,3,4-tetrahydro-pyrido[2,3-b]pyrazine;

(1-ethyl-propyl)-[2-methyl-8-(2,6-dimethyl-4-chloro-phenyl)-quinolin-4-yl]-amine;

8-(1-hydroxymethyl-propoxy)-6-methyl-4-(2,4,6-trimethyl-phenyl)-3,4-dihydro-1H-pyrido[2,3-b]pyrazin-2-one;

8-(1-hydroxymethyl-propylamino)-6-methyl-4-(2,4,6-trimethyl-phenyl)-3,4-dihydro-1H-pyrido[2,3-b]pyrazin-2-one;

8-(1-ethyl-propylamino)-6-methyl-4-(2,4,6-trimethyl-phenyl)-3,4-dihydro-1H-pyrido[2,3-b]pyrazin-2-one;

8-diethylamino-6-methyl-4-(2,4,6-trimethyl-phenyl)-3,4-dihydro-1H-pyrido-[2,3-b]pyrazin-2-one;

8-(ethyl-propyl-amino)-6-methyl-4-(2,4,6-trimethyl-phenyl)-3,4-dihydro-1H-pyrido[2,3-b]pyrazin-2-one;

8-(butyl-ethyl-amino)-6-methyl-4-(2,4,6-trimethyl-phenyl)-3,4-dihydro-1H-pyrido[2,3-b]pyrazin-2-one;

8-(1-hydroxymethyl-propoxy)-6-methyl-4-(2,4,6-trimethyl-phenyl)-1,2,3,4-tetrahydro-pyrido[2,3-b]pyrazine;

8-(1-hydroxymethyl-propylamino)-6-methyl-4-(2,4,6-trimethyl-phenyl)-1,2,3,4-tetrahydro-pyrido[2,3-b]pyrazine;

8-(1-ethyl-propylamino)-6-methyl-4-(2,4,6-trimethyl-phenyl)-1,2,3,4-tetrahydro-pyrido[2,3-b]pyrazine;

8-diethylamino-6-methyl-4-(2,4,6-trimethyl-phenyl)-1,2,3,4-tetrahydro-pyrido[2,3-b]pyrazine;

8-(ethyl-propyl-amino)-6-methyl-4-(2,4,6-trimethyl-phenyl)-1,2,3,4-tetrahydro-pyrido[2,3-b]pyrazine;

8-(butyl-ethyl-amino)-6-methyl-4-(2,4,6-trimethyl-phenyl)-1,2,3,4-tetrahydro-pyrido[2,3-b]pyrazine;

4-(1-hydroxymethyl-propoxy)-2-methyl-8-(2,4,6-trimethyl-phenyl)-quinoline;

4-(1-hydroxymethyl-propylamino)-2-methyl-8-(2,4,6-trimethyl-phenyl)-quinoline;

4-(1-ethyl-propylamino)-2-methyl-8-(2,4,6-trimethyl-phenyl)-quinoline;

4-diethylamino-2-methyl-8-(2,4,6-trimethyl-phenyl)-quinoline;

4-(ethyl-propyl-amino)-2-methyl-8-(2,4,6-trimethyl-phenyl)-quinoline;

4-(butyl-ethyl-amino)-2-methyl-8-(2,4,6-trimethyl-phenyl)-quinoline;

5-(1-hydroxymethyl-propoxy)-7-methyl-1-(2,4,6-trimethyl-phenyl)-1,4-dihydro-2H-3-oxa-1,8-diaza-naphthalene;

5-(1-hydroxymethyl-propylamino)-7-methyl-1-(2,4,6-trimethyl-phenyl)-1,4-dihydro-2H-3-oxa-1,8-diaza-naphthalene;

5-(1-ethyl-propylamino)-7-methyl-1-(2,4,6-trimethyl-phenyl)-1,4-dihydro-2H-3-oxa-1,8-diaza-naphthalene;

5-diethylamino-5-methyl-1-(2,4,6-trimethyl-phenyl)-1,4-dihydro-2H-3-oxa-1,8-diaza-naphthalene;

5-(ethyl-propyl-amino)-7-methyl-1-(2,4,6-trimethyl-phenyl)-1,4-dihydro-2H-3-oxa-1,8-diaza-naphthalene;

8-(butyl-ethyl-amino)-6-methyl-4-(2,4,6-trimethyl-phenyl)-1,4-dihydro-2H-3-oxa-1,8-diaza-naphthalene; 4-(2,4-dichlorophenyl)-5-methyl-2-[N-(1-(methoxymethyl)-1-(naphth-2-yl) methyl)-N-propylamino]thiazole;

oxalate of 4-(2,4-dichlorophenyl)-5-methyl-2-[N-(6-methoxylsoquinol-5-yl)-N-propylamino]thiazole;

oxalate of 4-(2-chloro-4-methoxyphenyl)-5-methyl-2-[N-(6-methylisoquinol-5-yl)-N-propylamino]thiazole;

4-(2-chloro-4-methoxyphenyl)-5-methyl-2-[N-(1-methoxycarbonylmethylindol-5-yl)-N-propylamino]thiazole;

oxalate of 4-(2-chloro-4-methoxyphenyl)-5-methyl-2-[N-(6-methoxylsoquinol-5-yl)-N-propylamino]thiazole;

oxalate of 4-(2-chloro-4-methoxyphenyl)-5-methyl-2-[N-(6-chloroisoquinol-5-yl)-N-propylamino]thiazole;

oxalate of 4-(2-chloro-4-methoxyphenyl)-5-methyl-2-[N-(6-methoxylsoquinol-5-yl)-N- propylamino]thiazole;

4-(2-chloro-4-methoxyphenyl)-5-methyl-2-[N-1-methoxynaphth-2-yl)-N-propylamino]thiazole;

oxalate of 4-(2-chloro-4-trifluoromethylphenyl)-5-methyl-2-[N-6-methoxylsoquinol-5-yl)-N-propylamino]thiazole;

chlorhydrate of 4-(2-chloro-4-methoxyphenyl)-5-methyl-2-[N-(2-ethoxynaphth-1-yl)-N- propylamino]thiazole;

chlorhydrate of 4-(2-chloro-4-methoxyphenyl)-5-methyl-2[N-(2,3-dimethylnaphth-1-yl)-N-propylamino]thiazole;

chlorhydrate of 4-(2-chloro-4-methoxyphenyl)-5-methyl-2-[N-(6-bromo-2-methoxynaphth-1-yl)-N-propylamino]thiazole;

chlorhydrate of 4-(2-chloro-4-methoxyphenyl)-5-methyl-2-[N-(2,6-dimethylnaphth-1-yl)-N-propylamino]thiazole;

chlorhydrate of 4-(2-chloro-4-methoxyphenyl)-5-methyl-2-[N-(1-(methoxymethyl)-1-(naphth-2-yl)methyl)-N-propylamino]thiazole;

chlorhydrate of 4-(2-chloro-4-methoxyphenyl)-5-methyl-2-[N-(1-(cyclopropyl)-1-(naphth-2-yl)methyl)-N-propylamino]thiazole;

3-(2,4-dichlorophenyl)-5-methyl-7(N-propyl-N-cyclopropanemethylamino)-pyrazolo[2,3-a]pyrimidine;

3-(2,4-dichlorophenyl)-5-methyl-7-(N-allyl-N-cyclopropanemethylamino)-pyrazolo[2,3-a]pyrimidine;

2-methylthio-3-(2,4-dichlorophenyl)-5-methyl-7-(N, N-diallylamino)-pyrazolo[2,3-a]pyrimidine;

2-methylthio-3-(2,4-dichlorophenyl)-5-methyl-7-(N-butyl-N-cyclopropane-methyl-amino)pyrazolo[2,3-a]pyrimidine;

2-methylthio-3-(2,4-dichlorophenyl)-5-methyl-7-(N-propyl-N-cyclopropane-methyl-amino)pyrazolo[2,3-a]pyrimidine;

2-methyl-3-(4-chlorophenyl)-5-methyl-7-(N, N-dipropylamino)-pyrazolo[2,3-a]pyrimidine;

3-[6-(dimethylamino)-3-pyridinyl-2,5-dimethyl-N,N-dipropylpyrazolo[2,3-a]pyrimidin-7-amine;

3-[6-(dimethylamino)-4-methyl-3-pyridinyl]-2,5-dimethyl-N,N-dipropyl-pyrazolo[2,3-a]pyrimidine-7-amine;

3-(2,4-dimethoxyphenyl)-2,5-dimethyl-7-(N-propyl-N-methyloxyethylamino)-pyrazolo(2,3-a]pyrimidine;

7-(N-diethylamino)-2,5-dimethyl-3-(2-methyl-4-methoxyphenyl-[1,5-a]-pyrazolopyrimidine;

7-(N-(3-cyanopropyl)-N-propylamino-2,5,dimethyl-3-(2,4-dimethylphenyl)-[1,5-a]-pyrazolopyrimidine;

[3,6-dimethyl-2-(2,4,6-trimethyl-phenoxy)-pyridin-4-yl]-(1-ethyl-propyl)-amine;

[2-(4-chloro-2,6-dimethyl-phenoxy)-3,6-dimethyl-pyridin-4-yl]-(1-ethyl-propyl)-amine;

cyclopropylmethyl-[3-(2,4-dimethyl-phenyl)-2,5-dimethyl-pyrazolo[1,5-a]pyrimidin-7-yl]-propyl-amine;

cyclopropylmethyl-[3-(2-methyl-4-chloro-phenyl)-2,5-dimethyl-pyrazolo[1,5-a]pyrimidin-7-yl]-propyl-amine;

cyclopropylmethyl-[3-(2,4-di-chloro-phenyl)-2,5-dimethyl-pyrazolo[1,5-a]pyrimidin-7-yl]-propyl-amine;

[3-(2-methyl-4-chloro-phenyl)-2,5-dimethyl-pyrazolo[1,5-a]pyrimidin-7-yl]-di-propyl-amine;

[2,5-dimethyl-3-(2,4-dimethyl-phenyl)-pyrazolo[1,5-a]pyrimidin-7-yl]-(1-ethyl-propyl)-amine;

[2,5-dimethyl-3-(2,4-dichloro-phenyl)-pyrazolo[1,5-a]pyrimidin-7-yl]-(1-ethyl-propyl)-amine;

4-(1-ethyl-propylamino)-6-methyl-2-(2,4,6-trimethyl-phenoxy)-nicotinic acid methyl ester;

3-[6-(dimethylamino)-4-methyl-3-pyridinyl]-2,5-dimethyl-N-propyl-N-cyclopropylmethyl-pyrazolo[2,3-a]pyrimidin-7-amine; and

3-[6-(dimethylamino)-4-methyl-3-pyridinyl]-2,5-dimethyl-N-ethyl-N-cyclopropylmethyl-pyrazolo[2,3-a]pyrimidin-7-amine.

Another group of useful CRF antagonists for use in the compositions, methods, and kits of the present invention are those wherein the CRF antagonist is a compound of the following formula, disclosed in WO 95/10506:

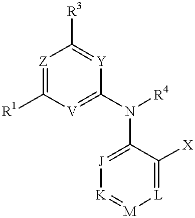

or a pharmaceutically acceptable salt or prodrug thereof, wherein Y is CR^{3a}, N, or CR²⁹;

when Y is CR^{3a} or N:

R¹ is independently selected at each occurrence from the group consisting of C₁-C₄ alkyl, C₂-C₄ alkenyl, C₂-C₄ alkynyl, halogen, C₁-C₂ haloalkyl, NR⁶R⁷S(O)ₙR⁸;

R³ is C₁-C₄ alkyl, aryl, C₃-C₆ cycloalkyl, C_{1=l -C}₂ haloalkyl, halogen, nitro, NR⁶R⁷, OR⁸, S(O)ₙR⁸C(═O)R⁹, C(═O)NR⁶R⁷, C(═S)NR⁶R⁷, —(CHR¹⁶)ₖ N R⁶R⁷, (CH₂)ₖOR⁸, C(═O)NR¹⁰CH(R¹¹)CO₂R¹², —C(OH )(R²⁵)(R^{25a}), —(CH₂)ₚS(O)ₙ-alkyl, —(CHR₁₆)R²⁵, —C(CN)(R²⁵)(R¹⁶) provided that R²⁵ is not —NH— containing rings, —C(═O)R²⁵, —CH(CO₂R¹⁶)₂, NR¹⁰C(═O)CH(R¹¹)NR¹⁰R¹², NR¹⁰CH(R¹¹)CO₂R¹²; substit C₁-C₄ alkyl, substituted C₂-C₄ alkenyl, substituted C₂-C₄ alkynyl, substituted C₁-C₄ alkoxy, aryl-(substituted C₁-C₄) alkyl, aryl-(substituted C₁-C₄) alkoxy, substituted C₃-C₆ cycloalkyl, amino-(substituted C₁-C₄)alkyl, substituted C₁-C₄ alkylamino, where substitution by R²⁷ can occur on any carbon containing substituent; 2-pyridinyl, imidazolyl, 3-pyridinyl, 4-pyridinyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-pheno-thiazinyl, 4-pyrazinyl, azetidinyl, phenyl, 1H-indazolyl, 2-pyrrolidonyl, 2H,6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azocinyl, azepinyl, benzofuranyl, benzothiophenyl, carbazolyl, chromanyl, chromenyl, cinnolinyl, decahydroquinolinyl, furazanyl, imidazolidinyl, indolinyl, indolizinyl, indolyl, isobenzofuranyl, isochromanyl, isoindolinyl, isoindolyl, isoquinolinyl, benzimidazolyl, isothiazolyl, isoxazolyl, morpholinyl, naphthyridinyl, octahydroisoquinolinyl, oxazolidinyl, oxazolyl, phenanthridinyl, phenanthrolinyl, phenazinyl, phenoxathiinyl, phenoxazinyl, phthalazinyl, piperazinyl, piperidinyl, pteridinyl, purinyl, pyranyl, pyrazolidinyl, pyrazolinyl, pyrazolyl, pyridazinyl, pyrimidinyl, pyrrolidinyl, pyrrolinyl, pyrrolyl, quinazolinyl, quinolinyl, quinoxalinyl, quinuclidinyl, β-carbolinyl, tetrahydrofuranyl, tetrahydroisoquinolinyl, tetrahydroquinolinyl, tetrazolyl, thianthrenyl, thiazolyl, thiophenyl, triazinyl, xanthenyl; or 1-tetrahydroquinolinyl or 2-tetrahydroisoquinolinyl either of which can be substituted with 0-3 groups chosen from keto and C₁-C₄ alkyl;

J, K, and L are independently selected at each occurrence from the group of N, CH, and CX′;

M is CR⁵ or N;

V is CR^{1a} or N;

Z is CR₂ or N;

R^{1a}, R², and R^{3a} are independently selected at each occurrence from the group consisting of hydrogen, halo, halomethyl, C₁-C₃ alkyl, and cyano;

R⁴ is (CH₂)ₘOR¹⁶, C₁-C₄ alkyl, allyl, propargyl, (CH₂)ₘR¹³, or —(CH₂)ₘOC(O)R¹⁶;

X is halogen, aryl, heteroaryl, S(O)₂R⁸, SR⁸, halomethyl, —(CH₂)ₚOR⁸, cyano, —(CHR¹⁶)ₚNR¹⁴R¹⁵, —C(═O)R⁸, C₁-C₆ alkyl, C₄-C₁₀ cycloalkylalkyl, C₁-C₁₀alkenyl, C₂-C₁₀alkynyl, C₂-C₁₀alkoxy, aryl-(C₂-C₁₀)-alkyl, C₃-C₆cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, or —C(═O)NR¹⁴R¹⁵, where substitution by R¹⁸ can occur on any carbon containing substituents;

X′ is independently selected at each occurrence from the group consisting of hydrogen, halogen, aryl, heteroaryl, S(O)ₙR⁸, halomethyl, —(CHR¹⁶)ₚOR⁸, cyano, —(CHR¹⁶)ₚNR¹⁴R¹⁵, C(═O)R⁸, C₁-C₆ alkyl, C₂-C₁₀alkenyl, C₂-C₁₀alkoxy, aryl-(C₁-C₁₀)-alkyl, C₃-C₆cycloalkyl, aryl-(C₁-C₁₀)-alkoxy, nitro, thio-(C₁-C₁₀)-alkyl, —C(═NOR¹⁶)-C₁-C₄-alkyl, —C(═NOR¹⁶)H, and —C(═O)NR¹⁴R¹⁵, where substitution by R¹⁶ can occur on any carbon containing substituents;

R⁵ is halo, —C(═NOR¹⁶)-C₁-C₄-alkyl, C₁-C₄alkyl, C₁-C₃ haloalkyl, —(CHR¹⁶)ₚOR⁸, —(CHR¹⁶)ₚS(O)ₙR⁸, —(CHR⁶)ₚNR¹⁴R¹⁵, C₃-C₆ cycloalkyl, C₂-C₁₀alkenyl, C₂-C₁₀alkynyl, aryl-(C₂-C₁₀)-akyl, aryl-(C₁-C₁₀)-alkoxy, cyano, C₃-C₆ cycloalkoxy, nitro, amino-(C₂-C₁₀)-alkyl, thio-(C₂-C₁₀)-alkyl, SOₙ(R⁸), C(═O)R⁸—C(═NOR¹⁰)H, or —C(═O)NR¹⁴R¹⁵, where substitution by R¹⁸ can occur on any carbon containing substituents;

R⁶ and R⁷ are independently selected at each occurrence from the group consisting of hydrogen, C₁-C₆ alkyl, C₃-C₁₀ cycloalkyl, C₁-C₆ alkoxy, (C₄-C₁₂)-cycloalkylalkyl, —(CH₂)ₖR¹³, (CHR¹⁶)ₚOR⁸, —(C₁-C₆alkyl)-aryl, heteroaryl, —S(O)_{z}—aryl or —(C₁-C₆alkyl)-heteroaryl or aryl, wherein the aryl or heteroaryl groups are optionally substituted with 1-3 groups selected from the group consisting of hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), cyano, and S(O)₂—(C₁-C₆-alkyl); or can be taken together to form —(CH₂)ₚA(CH₂)ᵣ—, optionally substituted with 0-3 R¹⁷; or, when considered with the commonly attached nitrogen, can be taken together to form a heterocycle, said heterocycle being substituted on carbon with 1-3 groups consisting of hydrogen, C₁-C₆ alkyl, hydroxy, or C₁-C₆ alkoxy;

A is CH₂, O, NR²⁵, C(═O), S(O)ₙ, N(C(═O)R¹⁷), N(R¹⁹), C(H)(NR¹⁴R¹⁵), C(H)(OR²⁰), C(H)(C(═O)R²¹), or N(S(O)ₙR ²¹);

R⁸ is independently selected at each occurrence from the group consisting of hydrogen; C₁-C₆ alkyl; —(C₄-C₁₂) cycloalkylalkyl; (CH₂)ₜR²²; C₃-C₁₀ cycloalkyl; —NR⁶R⁷; aryl; heteroaryl; —NR¹⁶(CH₂)ₙR⁶R⁷; —(CH₂)ₖR²⁵; and (CH₂)ₜheteroaryl or (CH₂)_{taryl}, either of which can optionally be substituted with 1-3 groups selected from the group consisting of hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), cyano, and S(O)₂(C₁-C₆-alkyl);

R⁹ is independently selected at each occurrence from R¹⁰, hydroxy, C₁-C₄ alkoxy, C₃-C₆ cycloalkyl, C₂-C₄ alkenyl, aryl substituted with 0-3 R¹⁸, and —(C₁-C₆ alkyl)-aryl substituted with 0-3 R¹⁸;

R¹⁰, R¹⁶, R₂₃, and R²⁴ are independently selected at each occurrence from hydrogen or C₁-C₄ alkyl;

R¹¹ is C₁-C₄ alkyl substituted with 0-3 groups chosen from the following: keto, amino, sulfhydryl, hydroxyl, guanidinyl, p-hydroxyphenyl, imidazolyl, phenyl, indolyl, and indolinyl, or, when taken together with an adjacent R¹⁰, are (CH₂)ₜ;

R¹² is hydrogen or an appropriate amine protecting group for nitrogen or an appropriate carboxylic acid protecting group for carboxyl;

R¹³ is independently selected at each occurrence from the group consisting of CN, OR¹⁹, SR¹⁹, and C₃-C₆ cycloalkyl;

R¹⁴ and R¹⁵ are independently selected at each occurrence from the group consisting of hydrogen, C₄-C₁₀, cycloalkyl-alkyl, and R₁₉;

R¹⁷ is independently selected at each occurrence from the group consisting of R¹⁰, C₁-C₄ alkoxy, halo, OR²³, SR²³, NR²³R²⁴, and (C₁-C₆) alkyl (C₁-C₄) alkoxy;

R¹⁸ is independently selected at each occurrence from the group consisting of R¹⁰, hydroxy, halogen, C₁-C₂ haloalkyl, C₁-C₄ alkoxy, C(═O)R²⁴, and cyano;

R¹⁹ is independently selected at each occurrence from the group consisting of C₁-C₆ alkyl, C₃-C₆ cycloalkyl, (CH₂)_{w}R²², and aryl substituted with 0-3 R¹⁸;

R²⁰ is independently selected at each occurrence from the group consisting of R¹⁰, C(═O)R³¹, and C₂-C₄ alkenyl;

R²¹ is independently selected at each occurrence from the group consisting of R¹⁰, C₁-C₄ alkoxy, NR²³R²⁴, and hydroxyl;

R₂₂ is independently selected at each occurrence from the group consisting of cyano, OR²⁴, SR²⁴, NR²³R²⁴, C₁-C₆ alkyl, C₃-C₆ cycloalkyl, —S(O)ₙR³¹, and —C(═O)R²⁵;

R²⁵, which can be optionally substituted with 0-3 R17, is independently selected at each occurrence from the group consisting of phenyl, pyrazolyl, imidazolyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-pheno-thiazinyl, 4-pyrazinyl, azetidinyl, 1H-indazolyl, 2-pyrrolidonyl, 2H,6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azocinyl, azepinyl, benzofuranyl, benzothiophenyl, carbazolyl, chromanyl, chromenyl, cinnolinyl, decahydroquinolinyl, furazanyl, indolinyl, indolizinyl, indolyl, isobenzofuranyl, isochromanyl, isoindolinyl, isoindolyl, isoquinolinyl benzimidazolyl, isothiazolyl, isoxazolyl, morpholinyl, naphthyridinyl, octahydroisoquinolinyl, oxazolidinyl, oxazolyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazolidinyl, pyridazinyl, pyridyl, pyrimidinyl, pyrrolidinyl, pyrrolinyl, pyrrolyl, quinazolinyl, quinolinyl, quinoxalinyl, quinuclidinyl, B-carbolinyl, tetrahydrofuranyl, tetrazolyl, thianthrenyl, thiazolyl, thiophenyl, triazinyl, xanthenyl; and 1-tetrahydroquinolinyl or 2-tetrahydroisoquinolinyl either of which can be substituted with 0-3 groups chosen from keto and C₁-C₄ alkyl;

R^{25a}, which can be optionally substituted with 0-3 R¹⁷, is independently selected at each occurrence from the group consisting of H and R²⁵;

R²⁷ is independently selected at each occurrence from the group consisting of C₁-C₃ alkyl, C₂-C₄ alkenyl, C₂-C₄ alkynyl, C₂-C₄ alkoxy, aryl, nitro, cyano, halogen, aryloxy, and heterocycle optionally linked through 0;

R³¹ is independently selected at each occurrence from the group consisting of C₁-C₄ alkyl, C₃-C₇ cycloalkyl, C₄-C₁o cycloalkyl-alkyl, and aryl-(C₁-C₄) alkyl;

k, m, and r are independently selected at each occurrence from 1-4;

n is independently, selected at each occurrence from 0-2,

p, q, and z are independently selected at each occurrence from 0-3;

t and w are independently selected at each occurrence from 1-6,

provided that when J is CX′ and K and L are both CH, and M is CR⁵, then

(A) when V and Y are N and Z is CH and R¹ and R³ are methyl,

(1) and R⁴ is methyl, then

(a) R⁵ can not be methyl when X is OH and X′ is H;

(b) R⁵ can not be —NHCH₃, or —N(CH₃)₂ when X and X′ are —OCH₃; and

(c) R⁵ can not be —N(CH₃)₂ when X and X′ are —OCH₂CH₃;

(2) and R⁴ is ethyl, then

(a) R⁵ can not be methylamine when X and X′ are —OCH₃;

(b) R⁵ can not be OH when X is Br and X′ is OH; and

(c) R⁵ can not be —CH₂OH or —CH₂N(CH₃)₂ when X is —SCH₃ and X′ is H;

(B) when V and Y are N, Z is CH, R⁴ is ethyl, R⁵ is iso-propyl, X is Br, X′ is H, and

(1) R¹ is CH₃, then

(a) R³ can not be OH, piperazin-1-yl, —CH₂,-piperidin-1-yl, —CH₂—(N-4-methylpiperazi n-1-yl), —C(O)NH-phenyl, —CO₂H, —CH₂O-(4-pyridyl), —C(O)NH₂, 2-indolyl, —CH₂O-(4-carboxyphenyl), —N(CH₂CH₃)(2-bromo-4-isopropylphenyl);

(2) R² is —CH₂CH₂CH₃ then R³ can not be —CH₂CH₂CH₃

(C) when V, Y and Z are N, R⁴ is ethyl, and

(1) R⁵ is iso-propyl, X is bromo, and X′ is H, then

(a) R³ can not be OH or —OCH₂CN when R¹ is CH₃ and

(b) R³ can not be —N(CH₃)₂ when R¹ is —N(CH₃)₂;

(2) R⁵ is —OCH₃, X is —OCH₃, and X′ is H, then R³ and R′ can not both be chloro;

further provided that when J, K, and L are all CH and M is CR⁵, then

(D) at least one of V, Y, and Z must be N;

(E) when V is CR^{1a}, Z and Y can not both be N;

(F) when Y is CR^{3a}, Z and V can not both be N;

(G) when Z is CR², V and Y must both be N;

(H) Z can be N only when both V and Y are N or when V is CR^{1a} and Y is CR^{3a};

(I) when V and Y are N, Z is CR², and R² is H or C₁-C₃ alkyl, and R⁴ is C₁-C₃ alkyl, R³ can not be 2-pyridinyl, indolyl, indolinyl, imidazolyl, 3-pyridinyl, 4-pyridinyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-phenothiazinyl, or 4-pyrazinyl;

(J) when V and Y are N; Z is CR²; R² is H or C₁-C₃ alkyl; R⁴ is C₁-C₄ alkyl, R⁵, X, and/or X′ are OH, halo, CF₃, C₁-C₄ alkyl, C₁-C₄ alkoxy, C₁-C₄ alkylthio, cyano, amino, carbamoyl, or C₁-C₄ alkanoyl; and R¹ is C₁-C₄ alkyl, then R⁴ can not be —NH(substituted phenyl) or —N(C₁-C₄ alkyl) (substituted phenyl);

and wherein, when Y is CR²⁹:

J, K, L, M, Z, A, k, m, n, p, q, r, t, w, R³, R¹⁰, R¹¹, R¹², R¹³, R¹⁶, R¹⁸, R¹⁹, R²¹, R²³, R²⁴, R²⁵, and R²⁷ are as defined above and R²⁵a, in addition to being as defined above, can also be C₁-C₄ alkyl, but

V is N;

R¹ is C₁-C₂ alkyl, C₂-C₄ alkenyl, C₂-C₄ alkynyl, C₂-C₄ alkoxy, halogen, amino methylamino, dimethylamino, aminomethyl, or N-methylaminomethyl;

R² is independently selected at each occurrence from the group consisting of hydrogen, halo, C₁-C₃, alkyl, nitro, amino, and —CO₂R¹⁰;

R₄ is taken together with R²⁹ to form a 5-membered ring and is —C(R²⁶)═ or —N═ when R²⁹ is —C(R³⁰)═ or —N═, or —CH(R²⁶)— when R²⁹ is —CH(R³⁰)—;

X is Cl, Br, I, S(O)ₙR⁸, OR⁸, halomethyl, —(CHR¹⁶)ₚOR⁸, cyano, —(CHR¹⁶)ₚNR¹⁴R¹⁵, C(═O)R⁸, C₁-C₆ alkyl, C₂-C₁₀ alkenyl, C₂-C₁₀ alkynyl, C₁-C₁₀, alkoxy, aryl-(C₁-C₁₀)-alkyl, C₃-C₆ cycloalkyl, aryl-(C₁-C₁₀)-alkoxy, nitro, thio-(C₁-C₁₀)-alkyl, —C(═NOR¹⁶)-C₁-C₄-alkyl, -C(═NOR¹⁶)H, or C(═O)NR¹⁴R¹⁵ where substitution by R¹⁸ can occur on any carbon containing substituents;

X′ is hydrogen, Cl, Br, I, S(O)ₙR⁸, —(CHR¹⁶)ₚOR⁸, halomethyl, cyano, —(CHR¹⁶)_{p—NR}¹⁴R¹⁵, C(═O)R⁸, C₁-C₆ alkyl, C₂-C₁₀alkenyl, C₂-C₁₀, alkynyl, C₂-C₁₀ alkoxy, aryl-(C₁-C₁₀)-alkyl, C₃-C₆ cycloalkyl, aryl-(C₂-C₁₀)-alkoxy, nitro, thio-(C₂-C₁₀)-alkyl, —C(═NOR¹⁶)-C₁-C₄-alkyl, —C(═NOR¹⁶)H, or C(═O)NR⁸R¹⁵ where substitution by R¹⁸ can occur on any carbon containing substituents;

R⁵ is halo, —C(═NOR¹⁶)-C₁-C₄-alkyl, C₁-C₆ alkyl, C₁-C₃ haloalkyl, C₁-C₆ alkoxy, (CHR¹⁶)OR⁵, (CHR¹⁶)ₚS(O)ₙR⁸, (CHR¹⁶)ₚNR¹⁴R¹⁵, C₃-C₆ cycloalkyl, C₂-C₁₀ alkenyl, C₂-C₁₀ alkynyl, aryl-(C₂-C₁₀)-alkyl, aryl-(C₁-C₁₀)-alkoxy, cyano, C₃-C₆ cycloalkoxy, nitro, amino-(C₁-C₁₀)-alkyl, thio-(C₁-C₁₀)-alkyl, SOₙ(R⁸), C(═O)R⁸, —C(═NOR¹⁶)H, or C(═O)NR⁸R¹⁵ where substitution by R¹⁸ can occur on any carbon containing substituents;

R⁶ and R⁷ are independently selected at each occurrence from the group consisting of hydrogen, C₁-C₆ alkyl, C₃-C₁₀ cycloalkyl, —(CH₂)ₖR¹³, (C₄-C₁₂)-cycloalkylalkyl, C₁-C₆ alkoxy, —(C₁-C₆ alkyl)-aryl, heteroaryl, aryl, —S(O)_{z}-aryl or —(C₁-C₆ alkyl)-heteroaryl or aryl wherein the aryl or heteroaryl groups are optionally substituted with 1-3 groups selected from hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), and cyano; or can be taken together to form —(CH₂)qA(CH₂)ᵣ—, optionally substituted with 0-3 R¹⁷; or, when considered with the commonly attached nitrogen, can be taken together to form a heterocycle, said heterocycle being substituted on carbon with 1-3 groups consisting of hydrogen, C₁-C₆ alkyl, hydroxy, or C₁-C₆ alkoxy;

R⁸ is independently selected at each occurrence from the group consisting of hydrogen, C₁-C₆ alkyl, —(C₄-C₁₂) cycloalkylalkyl, (CH₂)ₜR²², C₃-C₁₀ cycloalkyl, —(C₁-C₆ alkyl)-aryl, heteroaryl, —NR¹⁶, —N(CH₂)ₙNR⁶R⁷; —(CH₂)ₖR²⁵, —(C₁-C₆ alkyl)-heteroaryl or aryl optionally substituted with 1-3 groups selected from hydrogen, halogen, C₁-C₆ alkyl, C₁-C₆ alkoxy, amino, NHC(═O)(C₁-C₆ alkyl), NH(C₁-C₆ alkyl), N(C₁-C₆alkyl)₂, nitro, carboxy, CO₂(C₁-C₆ alkyl), and cyano;

R⁹ is independently selected at each occurrence from R¹⁰, hydroxy, C₁-C₄ alkoxy, C₃-C₆ cycloalkyl, C₂-C₄ alkenyl, and aryl substituted with 0-3 R¹⁸;

R¹⁴ and R¹⁵ are independently selected at each occurrence from the group consisting of hydrogen, C₁-C₆ alkyl, C₃-C₆ cycloalkyl, (CH₂)ₜR²², and aryl substituted with 0-3 R¹⁸;

R¹⁷ is independently selected at each occurrence from the group consisting of R¹⁰, C₁-C₄ alkoxy, halo, OR²³, SR²³, and NR²³R²⁴;

R²⁰ is independently selected at each occurrence from the group consisting of R¹⁰ and C(═O)R³¹;

R²² is independently selected at each occurrence from the group consisting of cyano, OR²⁴, SR²⁴, NR²³R²⁴, C₃-C₆ cycloalkyl, —S(O)nR³¹, and —C(═O)R²⁵;

R²⁶ is hydrogen or halogen;

R²⁸ is C₁-C₂, alkyl, C₂-C₄ alkenyl, C₂-C₄ alkynyl, hydrogen, C₁-C₂ alkoxy, halogen, or C₂-C₄ alkylamino;

R²⁹ is taken together with R⁴ to form a five membered ring and is: —CH(R³⁰)— when R⁴ is —CH(R²⁸)—, —C(R³⁰)═ or —N═when R⁴ is -C(R²⁸) ═or —N═;

R³⁰ is hydrogen, cyano, C₁-C₂ alkyl, C₁-C₂ alkoxy, halogen, C₁-C₂ alkenyl, nitro, amido, carboxy, or amino;

R³¹ is C₁-C₄ alkyl, C₃-C₇ cycloalkyl, or aryl-(C₁-C₄) alkyl; provided that when J, K, and L are all CH, M is CR⁵, Z is CH, R³ is CH₃, R²⁸ is H, R⁵ is isopropyl, X is Br, X′ is H, and R¹ is CH₃, then R³⁰ can not be H, —CO₂H, or —CH₂NH₂; and further provided that when J, K and L are all CH; M is CR⁵; Z is N; and

(A) R²⁹ is —C(R³⁰)═; then one of R²⁸ or R³⁰ is hydrogen;

(B) R²⁹ is N; then R³ is not halo, NH₂, NO₂, CF₃, CO₂H, CO₂-alkyl, alkyl, acyl, alkoxy, OH, or —(CH₂)ₘOalkyl;

(C) R²⁹ is N; then R²⁸ is not methyl if X or X′ are bromo or methyl and R⁵ is nitro; or

(D) R²⁹ is N; and R¹ is CH₃; and R³ is amino; then R⁵ is not halogen or methyl.

Preferred compounds of this group include those wherein:

i) V is N, R′ is methyl; and R³ is aryl, NR⁶R⁷, or OR⁸;

ii) V is N, R¹ is methyl; R³ is aryl, NR⁶R⁷, or OR⁸; and R⁴ is methyl or ethyl;

iii) V is N, R′ is methyl; R³ is aryl, NR⁶R⁷, or OR⁸; R⁴ is methyl or ethyl; and X is O(C₁-C₄ alkyl), Br, or C₁-C₄ alkyl;

iv) V is N, R¹ is methyl; R³ is aryl, NR⁶R⁷, or OR⁸; R⁴ is methyl, ethyl; X is OMe, Br, or (C₁-C₄ alkyl), M is C₁-C₄ alkyl, Br, Cl, or O(C₁-C₄ alkyl); and

v) V is N, R′ is methyl; R³ is aryl, NR⁶R⁷, OR⁸; or R⁴ is methyl, ethyl; X is OMe, Br, or C₁-C₄ alkyl, M is C₁-C₄ alkyl, Br, Cl, or O(C₁-C₄ alkyl); and L is CH, or N.

Another group of useful CRF antagonists for use in the compositions, methods, and kits of the present invention are those wherein the CRF antagonist is a compound of the following formula, disclosed in EP 0773023:

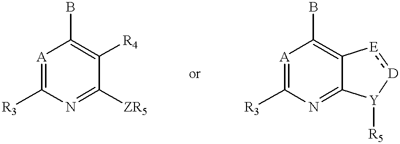

or a pharmaceutically acceptable salt thereof, wherein the dashed line represents an optional double bond;

A is —CR₇ or N;

B is —NR₁R₂, —CR₁R₂R₁₁, —C(═CR₁R₁₂)R₂, —NHCR₁₁R₁R₂, —OCR₁₁R₁R₂, —SCR₁₁R₁R₂, —CR₁₁R₂OR₁, —CR₁₁R₂SR₁, —C(S)R₂, —NHNR₁R₂, —CR₂R₁₁NHR, or —C(O)R₂;

D is N or —CR₁₀ when a double bond connects E and D and E is —CR⁴; —CR₁₀ when a double bond connects E and D and E is N; or —CR₈R₉, —CHR₁₀, —C═O, —C═S, —C═NH, or —C═NCH₃ when a single bond connects E and D;

E is —CR₄ or N when a double bond connects E and D, and E is —CR₄R₆ or —NR₆ when a single bond connects E and D;

Y is N or —CH;

Z is NH, O, S, —N(C₁-C₂ alkyl), or —CR₁₂R₁₃, wherein R₁₂ and R₁₃ are each, independently, hydrogen, trifluoromethyl, or methyl, or one of R₁₂ and R₁₃ is cyano and the other is hydrogen or methyl;

R₁ is hydrogen or C₁-C₆ alkyl which is optionally substituted with up to two substituents independently selected from hydroxy, cyano, nitro, fluoro, chloro, bromo, iodo, CF₃, C₁-C₄ alkoxy, —O—CO—(C₁-C₄ alkyl), —O—CO—NH(C₁-C₄ alkyl), —O—CO—N(C₁-C₄ alkyl)(C₁-C₂ alkyl), —NH(C₁-C₄ alkyl), —N(C₁-C₂ alkyl)(C₁-C₄ alkyl), —S(C₁-C₄ alkyl), —N(C₁-C₄alkyl)CO(C₁-C₄ alkyl), —NHCO(C₁-C₄ alkyl), —CO₂(C₁-C₄ alkyl), —CONH(C₁C₄ alkyl), —CON(C₁-C₄ alkyl)(C₁-C₂ alkyl), (C₁-C₄ alkyl)sulfinyl, (C₁-C₄ alkyl)sulfonyl, and (C₁-C₄ alkyl)sulfanyl, and wherein said C₁-C₆ alkyl, C₁-C₄ alkoxy, and C₁-C₄ alkyl moieties in the foregoing R₁ groups optionally contain one double or triple bond;

R₂ is C₁-C₆ alkyl, heteroaryl, aryl, heteroaryl (C₁-C₄ alkyl), or aryl (C₁-C₄ alkyl), wherein said aryl and the aryl moiety of said (aryl)C₁-C₄ alkyl are selected from the group consisting of phenyl and naphthyl, and said heteroaryl and the heteroaryl moiety of said (heteroaryl)C₁-C₄ alkyl is selected from the group consisting of thienyl, benzothienyl, pyridyl, thiazolyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, and benzoxazolyl; or R² is C₃-C₈ cycloalkyl or (C₃-C₈ cycloalkyl)C₁-C₆ alkyl, wherein one or two of the ring carbons of said cycloalkyl having at least 4 ring members and the cycloalkyl moiety of said (C₃-C₈ cycloalkyl)C₁-C₆ alkyl having at least 4 ring members is optionally replaced by an oxygen or sulfur atom or by —NR₁₄ wherein R₁₄ is hydrogen or C₁-C₄ alkyl; and wherein each of the foregoing R₂ groups is optionally substituted by up to three substituents independently selected from chloro, fluoro, and C₁-C₄ alkyl, or by one substituent selected from bromo, iodo, cyano, nitro, C₁-C₆ alkoxy, —O—CO—(C₁-C₄ alkyl), —O—CO—N(C₁-C₄ alkyl)(C₁-C₂ alkyl), —CO₂(C₁-C₄ alkyl), (C₁-C₄ alkyl)sulfanyl, (C₁-C₄ alkyl)sulfinyl, and (C₁-C₄ alkyl)sulfonyl, and wherein said C₁-C₄ alkyl and C₁C₆ alkyl moieties of the foregoing R₂ groups optionally contain one carbon-carbon double or triple bond;

or R¹ and R² of said —NR₁R₂ and said —CR₁R₂R₁₁ are taken together to form a saturated or partially saturated 5- to 8-membered ring, wherein said ring optionally contains one or two carbon-carbon double bonds, and wherein one or two of the ring carbons is optionally replaced by a heteroatom selected from O, S, and N;

R₃ is hydrogen, C₁-C₆ alkyl, fluoro, chloro, bromo, iodo, hydroxy, amino, SH, —NH(C₁-C₄ alkyl), —N(C₁-C₄ alkyl)(C₁-C₂ alkyl), —CH₂OH, —CH₂OCH₃, —O(C₁-C₄ alkyl), (C₁-C₄ alkyl)sulfanyl, (C₁-C₄ alkyl)sulfonyl, or (C₁-C₄ alkyl)sulfinyl, wherein said C₁-C₆ alkyl and C₁-C₄ alkyl moieties of the foregoing R₃ groups optionally contain one double or triple bond and are optionally substituted by from one to three substituents independently selected from hydroxy, amino, C₁-C₃ alkoxy, —NH(C₁-C₂ alkyl), —N(C₁-C₂ alkyl)₂, —NHCOCH₃, fluoro, chloro, and C₁-C₃ thioalkyl;

R₄ is hydrogen, C₁-C₆ alkyl, fluoro, chloro, bromo, iodo, C₁-C₆ alkoxy, formyl, trifluoromethoxy, —CH₂OCH₃, —CH₂OCH₂CH₃, —CH₂CH₂OCH₃, —CH₂CF₃, CF₃, amino nitro, —NH(C₁-C₄ alkyl), —N(CH₃)₂, —NHCOCH₃, —NHCONHCH₃, (C₁-C₄ alkyl)sulfanyl, (C₁-C₄ alkyl)sulfinyl, (C₁-C₄ alkyl)sulfonyl, cyano, hydroxy, —CO(C₁-C₄ alkyl), —CHO, or —CO₂(C₁-C₄ alkyl), wherein said C₁-C₆ alkyl, C₁-C₆ alkoxy, and C₁-C₄ alkyl moieties of the foregoing R₄ groups optionally contain one double or triple bond and are optionally substituted with one substituent selected from hydroxy, amino, —NHCOCH₃, —NH(C₁-C₂ alkyl), —N(C₁-C₂ alkyl)₂, —CO₂(C₁-C₄ alkyl), —CO(C₁-C₄ alkyl), C₁-C₃ alkoxy, (C₁-C₃ alkyl)sulfanyl, fluoro, chloro, cyano, and nitro;

R₅ is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzoisothiazolyl, thiazolyl, isoxazolyl, benzisoxazolyl, benzimidazolyl, triazolyl, pyrazolyl, pyrrolyl, indolyl, azaindolyl, benzoxazolyl, oxazolyl, pyrrolidinyl, thiazolidinyl, morpholinyl, pyridinyl, tetrazolyl, or a 3- to 8-membered cycloalkyl ring or a 9- to 12-membered bicycloalkyl ring system, wherein said cycloalkyl ring and said bicycloalkyl ring system optionally contain one or two of O, S, or —N—G wherein G is hydrogen, C₁-C₄ alkyl, C₁-C₄ alkanoyl, phenyl, or benzyl, wherein each of the above R₅ groups is optionally substituted by up to three substituents independently selected from fluoro, chloro, C₁-C₆ alkyl, C₁-C₆ alkoxy, and trifluoromethyl, or one substituent selected from bromo, iodo, cyano, nitro, amino, —NH(C₁-C₄ alkyl), —N(C₁-C₄ alkyl)(C₁-C₂ alkyl), —CO₂(C₁-C₄ alkyl), —CO(C₁-C₄ alkyl), —SO₂NH(C₁-C₄ alkyl), —SO₂N(C₁-C₄ alkyl)(C₁-C₂ alkyl), —SO₂NH₂, —NHSO₂(C₁-C₄ alkyl), —S(C₁-C₄ alkyl), and —SO₂(C₁-C₄ alkyl), wherein said C₁-C₄ alkyl and C₁-C₆ alkyl moieties of the foregoing R₅ groups optionally contain one double or triple bond and are optionally substituted by one or two substituents independently selected from fluoro, chloro, hydroxy, amino, methylamino, dimethylamino, and acetyl;

R₆ is hydrogen or C₁-C₆ alkyl, wherein said C₁-C₆ alkyl is optionally substituted by a single hydroxy, methoxy, ethoxy, or fluoro group;

R₇ is hydrogen, C₁-C₄ alkyl, fluoro, chloro, bromo, iodo, cyano, hydroxy, C₁-C₄ alkoxy, —CO(C₁-C₄ alkyl), —CO₂(C₁-C₄ alkyl), —OCF₃, CF₃, —CH₂OH, —CH₂OCH₃, or —CH₂OCH₂CH₃;

R₈ and R₉ are each, independently, hydrogen, hydroxy, methyl, ethyl, methoxy, or ethoxy;

or R₈ and R₉ together form an oxo (═O) group;

R₁₀ is hydrogen, C₁-C₆ alkyl, fluoro, chloro, bromo, iodo, C₁-C₆ alkoxy, formyl, amino, —NH(C₁-C₄ alkyl), —N(C₁-C₄ alkyl)(C₁-C₂ alkyl), cyano, carboxy, amido, or —SOₙ(C₁-C₄ alkyl) wherein n is 0, 1, or 2, wherein said C₁-C₆ alkyl and C₁-C₄ alkyl moieties of the foregoing R₁₀ groups are optionally substituted by one of hydroxy, trifluoromethyl, amino, carboxy, amido, —NHCO(C₁-C₄ alkyl), —NH(C₁-C₄ alkyl), —N(C₁-C₄ alkyl)(C₁-C₂ alkyl), —CO₂(C₁-C₄ alkyl), C₁-C₃ alkoxy, C₁-C₃ thioalkyl, fluoro, bromo chloro, iodo, cyano, or nitro; and

R₁₁ is hydrogen, hydroxy, fluoro, or methoxy.

Another group of preferred CRF antagonists for use in the compositions, methods, and kits of the present invention are those wherein the CRF antagonist is a compound selected from the group consisting of:

4-(1-ethyl-propoxy)-3,6-dimethyl-2-(2,4,6-trimethylphenoxy)-pyridine;

4-(1-ethylpropoxy)-2,5-dimethyl-6-(2,4,6-trimethylphenoxy)-pyrimidine;

[4-(1-ethyl-propoxy)-3,6-dimethyl-pyridin-2-yl]-(2,4,6-trimethylphenyl)-amine;

3-{(4-methyl-benzyl)-[3,6-dimethyl-1-(2,4,6-trimethylphenyl)-1H-pyrazolo[3,4-d]pyrimidin-4-yl]-amino}-propan-1-ol;

propyl-ethyl-[3,6-dimethyl-1-(2,4,6-trimethylphenyl)-1H-pyrazolo[3,4-d]pyrimidin-4-yl]-amine;

ethyl-n-propyl-[2,5-dimethyl-7-(2,4,6-trimethylphenyl)-7H-pyrrolo[2,3-d]pyrimidin-4-yl]amine;

2-{N-n-butyl-N-[2,5-dimethyl-7-(2,4,6-trimethylphenyl)-7H-pyrrolo[2,3-d]pyrimidin-4-yl]amino}-ethanol;

[3,6-dimethyl-1-(2,4,6-trimethylphenyl)-1H-pyrazolo[3,4,b]pyridin-4-yl]-(1-methoxymethylpropyl)-amine;

4-(1-ethylpropoxy)-2,5-dimethyl-7-(2,4,6-trimethylphenyl)-7H-pyrrolo[2,3-b]pyridine;

2,5,6-trimethyl-7-(1-propylbutyl)-4-(2,4,6-trimethylphenoxy)-7H-pyrrolo[2,3-d]pyrimidine;

1-(1-ethylpropyl)-6-methyl-4-(2,4,6-trimethylphenoxy)-1,3-dihydro-imidazo[4,5-c]pyridin-2-one;

1-(1-ethyl-propyl)-4,7-dimethyl-5-(2,4,6-trimethyl-phenoxy)-1,4-dihydro-2H-pyrido[3,4-b]pyrazin-3-one;

1-(1-ethyl-propyl)-4,7-dimethyl-5-(2,4,6-trimethyl-phenoxy)-1,2,3,4-tetrahydro-pyrido[3,4-b]pyrazine;

1-(1-ethyl-propyl)-7-methyl-2-oxo-5-(2,4,6-trimethyl-phenoxy)-1,2,3,4-tetra-hydro-[1,6]naphthyridine-3-carboxylic acid isopropyl ester;

1-(1-ethyl-propyl)-7-methyl-5-(2,4,6-trimethyl-phenoxy)-1,4-dihydro-2H-3-oxa-1,6-diaza-naphthalene;

(1-ethyl-propyl)-[5-methyl-3-(2,4,6-trimethyl-phenyl)-pyrazolo[1,5-a]pyrimidin-7-yl]-amine;

7-(1-ethyl-propoxy)-2,5-dimethyl-3-(2,4,6-trimethyl-phenyl)-pyrazolo[1,5-alpyrimidine;

4-(1-ethyl-propoxy)-2,5-dimethyl-7-(2,4,6-trimethyl-phenyl)-5H-pyrrolo[3,2-d]pyrimidine;

4-(butyl-ethyl-amino)-2,6-dimethyl-8-(2,4,6-trimethyl-phenyl)-5,8-dihydro-6H-pyrido[2,3-d]pyrimidin-7-one;

8-(1-ethyl-propoxy)-6-methyl-4-(2,4,6-t methyl-phenyl)-1,2,3,4-tetrahydro-pyrido[2,3-b]pyrazine;

4-(1-ethyl-propoxy)-2-methyl-8-(2,4,6-trimethyl-phenyl)-quinoline;

(1-ethyl-propyl)-[2-methyl-8-(2,4,6-trimethyl-phenyl)-quinolin-4-yl]-amine;

(propyl-ethyl)-[2-methyl-8-(2,4,6-trimethyl-phenyl)-5,6,7,8-tetrahydro-pyrido-[2,3-d]pyrimidin-4-yl]-amine;

(1-ethyl-propoxy)-2-methyl-8-(2,4,6-trimethyl-phenyl)-5,6,7,8-tetrahydro-pyrido[2,3-d]pyrimidine;

8-(1-hydroxymethyl-propylamino)-6-methyl-4-(2,4,6-trimethyl-phenyl)-3,4-dihydro-1H-pyrido[2,3-b]pyrazin-2-one;

4-(1-hydroxymethyl-propylamino)-2-methyl-8-(2,4,6-trimethyl-phenyl)-quinoline;

5-(1-hydroxymethyl-propylamino)-7-methyl-1-(2,4,6-trimethyl-phenyl)-1,4-dihydro-2H-3-oxa-1,8-diaza-naphthalene;

[3,6-dimethyl-2-(2,4,6-trimethyl-phenoxy)-pyridin-4-yl]-(1-ethyl-propyl)-amine;

cyclopropylmethyl-[3-(2,4-dimethyl-phenyl)-2,5-dimethyl-pyrazolo[1,5-a]pyrimidin-7-yl]-propyl-amine;

[2,5-dimethyl-3-(2,4-dimethyl-phenyl)-pyrazolo[1,5-a]pyrimidin-7-yl]-(1-ethyl-propyl)-amine;

3-[6-(dimethylamino)-3-pyridinyl-2,5-dimethyl-N,N-dipropylpyrazolo[2,3-a]pyrimidin-7-amine;

3-[6-(dimethylamino)-4-methyl-3-pyridinyl]-2,5-dimethyl-N,N-dipropyl-pyrazolo[2,3-a]pyrimidine-7-amine;

3-(2,4-dimethoxyphenyl)-2,5-dimethyl-7-(N-propyl-N-methyloxyethylamino)-pyrazolo(2,3-a)pyrimidine;

7-(N-diethylamino)-2,5-dimethyl-3-(2-methyl-4-methoxyphenyl-[1,5-a]-pyrazolopyrimidine; and

7-(N-(3-cyanopropyl)-N-propylamino-2,5,dimethyl-3-(2,4-dimethylphenyl)-[1,5-a]-pyrazolopyrimidine.

A group of preferred growth hormones or growth hormone secretagogues for use in the compositions, methods, and kits of the present invention are those wherein the growth hormone or growth hormone secretagogue is a growth hormone.

A group of preferred growth hormone secretagogues for use in the compositions, methods, and kits of the present invention are those wherein the growth hormone secretagogue is a compound of formula IV:

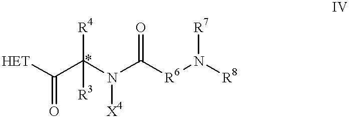

or a stereoisomeric mixture thereof, a diastereomerically enriched, diastereomerically pure, enantiomerically enriched, or enantiomerically pure isomer thereof, or a prodrug of such compound, mixture, or isomer thereof, or a pharmaceutically acceptable salt of the compound, mixture, isomer, or prodrug, wherein:

HET is a heterocyclic moiety selected from the group consisting of

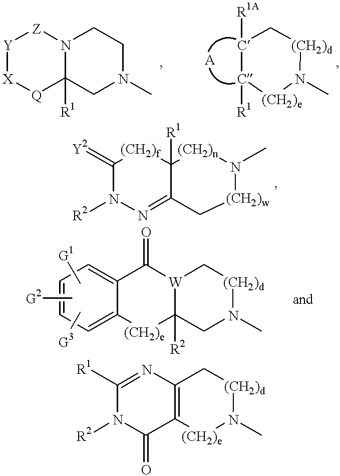

d is 0, 1, or 2;

e is 1 or 2;

f is 0 or 1;

n and w are 0, 1, or 2, provided that n and w cannot both be 0 at the same time;

y² is oxygen or sulfur;

A is a divalent radical, wherein the left hand side of the radical as shown below is connected to C″ and the right hand side of the radical as shown below is connected to C′, selected from the group consisting of —NR²—CO—NR²—, —NR²—SO₂—NR²—, —O—CO—NR²—, —NR²—CO₂—, —CO—NR²—CO—, —CO—NR²—C(R⁹R¹⁰)—, —C(R⁹R¹⁰)—NR²—CO—, —C(R⁹R¹⁰)—C(R⁹R¹⁰)—C(R⁹R¹⁰)—, —SO₂—C(R⁹R¹⁰)—C(R⁹R¹⁰), —C(R⁹R¹⁰)—O—CO—, —C(R⁹R¹⁰)—O—C(R⁹R¹O)—, —NR² CO—C(R⁹R¹⁰)—, —O—CO—C(R⁹R¹⁰)—, —C(R⁹R¹⁰)—CO—NR²—, —CO—NR²—CO—, —C(R⁹R¹⁰)—CO²—, —CO—NR²—C(R⁹R¹⁰)—C(R⁹R¹⁰—, —CO₂—C(R⁹R¹⁰)—, —C(R⁹R¹⁰)—C(R⁹R¹⁰)—C(R⁹R¹⁰)—C(R⁹R¹⁰)—, —SO₂NR²—C(R⁹R¹⁰)—C(R⁹R¹⁰)—, —C(R⁹R¹⁰)—C(R⁹R¹⁰)—NR²—CO—, —C(R⁹R¹⁰)—C(R⁹R¹⁰)—O—CO—, —NR²—CO—C(R⁹R¹⁰)—C(R⁹R¹⁰)—, —NR²—SO₂—C(R⁹R¹⁰)—C(R⁹R¹⁰)—, —O—CO—C(R⁹R¹⁰)—C(R⁹R¹⁰)—, —C(R⁹R¹⁰)—C(R⁹R¹⁰)—CO—NR²—, —C(R⁹R¹⁰)—C(R⁹R¹⁰)—CO—, —C(R⁹R¹⁰)—NR²—CO₂—, —C(R⁹R¹⁰)—O—CO—NR², —C(R⁹R¹⁰)—NR²—CO—NR²—, —NR²—CO₂—C(R⁹R¹⁰)—, —NR²—CO—NR²—C(R⁹R¹⁰)—, —NR—SO₂—NR²—C(R⁹R¹⁰)—, —O—CO—NR²—C(R⁹R¹⁰)—, —CO—N═C(R¹¹)—NR²—, —CO—NR²—C(R¹¹)═N—, —C(R⁹R¹⁰)—NR¹²—C(R⁹R¹⁰)—, —NR¹²—C(R⁹R¹⁰)—, —NR¹²—C(R⁹R¹⁰)—C(R⁹R¹⁰)—, —CO₂—C(R⁹R¹⁰)—C(R⁹R¹⁰)—, —NR²—C(R¹¹)═N—CO—, —C(R⁹R¹⁰)—C(R⁹R¹⁰)—N(R¹²)—, —C(R⁹R¹⁰)—NR¹², —N═C(R¹¹)—NR²—CO—, —C(R⁹R¹⁰)—C(R⁹R¹⁰)—NR²—SO₂—, —C(R⁹R¹⁰)—C(R⁹R¹⁰)—SO₂—NR²—, —C(R⁹R¹⁰)—C(R⁹R¹⁰)—CO₂—, —C(R⁹R¹⁰)—SO₂—C(R⁹R¹⁰)—, —C(R⁹R¹⁰)—C(R⁹R¹⁰)—SO₂—, —O—C(R⁹R¹⁰)—C(R⁹R¹⁰)—, —C(R⁹R¹⁰)—C(R⁹R¹⁰)—O—, —(R⁹R¹⁰)—C(R⁹R¹⁰)—, —CO—C(R⁹R¹⁰)—C(R⁹R¹⁰)—, and —C(R⁹R¹⁰)—NR²—SO₂—NR²—;

Q is a covalent bond or CH₂;

W is CH or N;

X is CR⁹R¹⁰, C═CH₂, or C═O;

Y is CR⁹R¹⁰, O, or NR²;

Z is C═O, C═S, or SO₂;

G¹is hydrogen, halo, hydroxy, nitro, amino, cyano, phenyl, carboxyl, —CONH₂, —C₁-C₄ alkyl optionally independently substituted with one or more phenyl, one or more halogen, or one or more hydroxy groups, —C₁-C₄ alkoxy optionally independently substituted with one or more phenyl, one or more halogen, or one or more hydroxy groups, —C₁-C₄ alkylthio, phenoxy, —CO₂-(C₁-C₄ alkyl), N,N-di-(C₁-C₄ alkylamino), —C₂-C₆ alkenyl optionally independently substituted with one or more phenyl, one or more halogen, or one or more hydroxy groups, —C₂-C₆ alkynyl optionally independently substituted with one or more phenyl, one or more halogen, or one or more hydroxy groups, —C₃-C₆ cycloalkyl optionally independently substituted with one or more C₁-C₄ alkyl groups, one or more halogen, or one or more hydroxy groups, —C₁-C₄ alkylamino carbonyl, or di-C₁-C₄ alkylamino) carbonyl;

G² and G³ are each independently selected from the group consisting of hydrogen, halo, hydroxy, —C₁-C₄ alkyl optionally independently substituted with one to three halo groups, and —C₁-C₄ alkoxy optionally independently substituted with one to three halo groups;

R¹ is hydrogen, —CN, —(CH₂)_{q}NX⁶COX⁶, —(CH₂)_{q}NX⁶CO(CH₂)ₜ—A¹, —(CH₂)_{q}NX⁶SO₂(CH₂)ₜ—A¹, —(CH₂)_{q}NX⁶SO₂X⁶, —(CH₂)_{q}NX⁶CONX⁶(CH₂)ₜ—A¹, —(CH₂)_{q}NX⁶CONX⁶X⁶, —(CH₂)_{q}CONX⁶X⁶, (CH₂)_{q}, CONX⁶(CH₂)ₜ—A¹, —(CH₂)_{q}CO₂X⁶, —(CH₂)_{q}CO₂(CH₂)ₜ—A¹, —(CH₂)_{q}OX⁶, —(CH₂)_{q}OCOX⁶, —(CH₂)_{q}OCO(CH₂)ₜ—A¹, —(CH₂)_{q}OCONX⁶(CH₂)ₜ—A¹, —(CH₂)_{q}OCONX⁶X⁶, —(CH₂)_{q}COX⁶, —(CH₂)_{q}CO(CH₂)ₜ—A¹, —(CH₂)_{q}NX⁶CO₂X⁶, —(CH₂)_{q}NX⁶SO₂NX⁶X⁶, —(CH₂)_{q}SOₘX⁶, —(CH₂)_{q}SOₘ(CH₂)ₜ—A¹, —C₁-C₁₀ alkyl, —(CH₂)ₜ—A¹, —(CH₂)_{q}—(C₃-C₇ cycloalkyl), —(CH₂)_{q}—Y¹—(C₁-C₆ alkyl), —(CH₂)_{q}—, Y¹—(CH₂)ₜ—A¹, or —(CH₂)_{q}—Y¹—(CH₂)ₜ—(C₃-C₇ cycloalkyl);

wherein the alkyl and cycloalkyl groups in the definition of R¹ are optionally substituted with C₁-C₄ alkyl, hydroxy, C₁-C₄ alkoxy, carboxyl, —CONH₂, —SOₘ—(C₁-C₆ alkyl), —CO₂—(C₁-C₄ alkyl) ester, 1H-tetrazol-5-yl, or 1, 2, or 3 fluoro groups;

Y¹ is O, SOₘ, —CONX⁶—, —CH═CH—, —C≡C—, —NX⁶CO—, —CONX⁶—, —CO₂—, —OCONX⁶— or —OCO—;

q is 0, 1, 2, 3, or 4;

t is 0, 1, 2, or 3;

said (CH₂)_{q} group and (CH₂)ₜ group in the definition of R¹ are optionally independently substituted with hydroxy, C₁-C₄ alkoxy, carboxyl, —CONH₂, —SOₘ—(C₁-C₆ alkyl), —CO₂—(C₁-C₄ alkyl) ester, 1H-tetrazol-5-yl, 1, 2, or 3 fluoro groups, or 1 or 2 C₁-C₄ alkyl groups;

R^{1A} is selected from the group consisting of hydrogen, F, Cl, Br, I, C₁-C₆ alkyl, phenyl-(C₁-C₃ alkyl), pyridyl-(C₁-C₃ alkyl), thiazolyl-(C₁-C₃ alkyl), and thienyl-(C₁-C₃ alkyl), provided that R^{1A} is not F, Cl, Br, or I when a heteroatom is vicinal to C″;

R² is hydrogen, C₁-C₈ alkyl, —(C₀-C₃ alkyl)-(C₃-C₈ cycloalkyl), —(C₁-C₄ alkyl)-A¹, or A¹, wherein the alkyl groups and the cycloalkyl groups in the definition of R² are optionally substituted with hydroxy, —CO₂X⁶, —CONX⁶X⁶, —NX⁶X⁶, —SOₘ(C₁-C₆ alkyl), —COA¹, —COX⁶, CF₃, CN, or 1, 2, or 3 independently selected halo groups;

R³ is selected from the group consisting of A¹, C₁-C₁₀ alkyl, —(C₁-C₆ alkyl)-A¹, —(C₁-C₆ alkyl)-(C₃-C₇ cycloalkyl), —(C₁-C₅ alkyl)-X¹—(C₁-C₅ alkyl), —(C₁-C₅ alkyl)-X¹—(C₀-C₅ alkyl)-A¹, and —(C₁-C₅ alkyl)-X¹—(C₁-C₅ alkyl)-(C₃-C₇ cycloalkyl);

wherein the alkyl groups in the definition of R³ are optionally substituted with —SOₘ(C₁-C₆ alkyl), —CO₂X³, 1, 2, 3, 4, or 5 independently selected halo groups, or 1, 2, or 3 independently selected —OX³ groups;

X¹ is O, SOₘ, —NX²CO—, —CONX²—, —OCO—, —CO₂—, —CX²═CX²—, —NX²CO₂—, —OCONX²—, or —C═C—;

R⁴ is hydrogen, C₁-C₆ alkyl, or C₃-C₇ cycloalkyl, or R⁴ taken together with R³ and the carbon atom to which they are attached form C₅-C₇ cycloalkyl, C₅-C₇ cycloalkenyl, a partially saturated or fully saturated 4- to 8-membered ring having 1to 4 heteroatoms independently selected from the group consisting of oxygen, sulfur, and nitrogen, or a bicyclic ring system consisting of a partially saturated or fully saturated 5- or 6-membered ring, fused to a partially saturated, fully unsaturated, or fully saturated 5- or 6-membered ring, optionally having 1to 4 heteroatoms independently selected from the group consisting of nitrogen, sulfur, and oxygen;

X⁴ is hydrogen or C₁-C₆ alkyl, or X⁴ is taken together with R⁴ and the nitrogen atom to which X⁴ is attached and the carbon atom to which R⁴ is attached and form a five to seven membered ring;

R⁶ is a bond or is

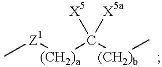

wherein a and b are each independently 0, 1, 2, or 3;

X⁵ and X^{5a} are each independently selected from the group consisting of hydrogen, CF₃, A¹, and C₁-C₆ alkyl optionally substituted with A¹, OX², —SOₘ—(C₁-C₆ alkyl), —CO₂X², C₃-C₇ cycloalkyl, —NX²X², or —CONX²X²;

or the carbon bearing X⁵ or X^{5a} forms one or two alkylene bridges with the nitrogen atom bearing R⁷ and R⁸ wherein each alkylene bridge contains 1 to 5 carbon atoms, provided that when one alkylene bridge is formed then only one of X⁵ or X^{5a} is on the carbon atom and only one of R⁷ or R⁸ is on the nitrogen atom, and further provided that when two alkylene bridges are formed then X⁵ and X^{5a} cannot be on the carbon atom and R⁷ and R⁸ cannot be on the nitrogen atom;

or X⁵ taken together with X^{5a} and the carbon atom to which they are attached form a partially saturated or fully saturated 3- to 7-membered ring, or a partially saturated or fully saturated 4- to 8-membered ring having 1 to 4 heteroatoms independently selected from the group consisting of oxygen, sulfur, and nitrogen;

or X⁵ taken together with X^{5a} and the carbon atom to which they are attached form a bicyclic ring system consisting of a partially saturated or fully saturated 5- or 6-membered ring, optionally having 1 or 2 heteroatoms independently selected from the group consisting of nitrogen, sulfur, and oxygen, fused to a partially saturated, fully saturated, or fully unsaturated 5- or 6-membered ring, optionally having 1 to 4 heteroatoms independently selected from the group consisting of nitrogen, sulfur, and oxygen;

Z¹is a bond, O, or N—X², provided that when a and b are both 0 then Z¹is not N—X² or O;

R⁷ and R⁸ are each independently hydrogen or C₁-C₆ alkyl optionally independently substituted with A¹, —CO₂—(C₁-C₆ alkyl), —SOₘ(C₁-C₆ alkyl), 1 to 5 halo groups, 1 to 3 hydroxy groups, 1 to 3 —O—CO(C₁-C₁₀ alkyl) groups, or 1 to 3 C₁-C₆ alkoxy groups; or

R⁷ and R⁸ can be taken together to form —(CH₂)ᵣ—L—(CH₂)ᵣ—, wherein L is CX²X², SOₘ, or NX²;

R⁹ and R¹⁰ are each independently selected from the group consisting of hydrogen, fluoro, hydroxy, and C₁-C₅ alkyl optionally independently substituted with 1-5 halo groups;

R¹¹ is selected from the group consisting of C₁-C₅ alkyl and phenyl optionally substituted with 1-3 substituents each independently selected from the group consisting of C₁-C₅ alkyl, halo, and C₁-C₅ alkoxy;

R¹² is selected from the group consisting of C₁-C₅ alkylsulfonyl, C₁-C₅ alkanoyl, and C₁-C₅ alkyl wherein the alkyl portion is optionally independently substituted by 1-5 halo groups;

A¹ for each occurrence is independently selected from the group consisting of C₅-C₇ cycloalkenyl, phenyl, a partially saturated, fully saturated, or fully unsaturated 4- to 8-membered ring optionally having 1 to 4 heteroatoms independently selected from the group consisting of oxygen, sulfur, and nitrogen, and a bicyclic ring system consisting of a partially saturated, fully unsaturated, or fully saturated 5- or 6-membered ring, optionally having 1 to 4 heteroatoms independently selected from the group consisting of nitrogen, sulfur, and oxygen, fused to a partially saturated, fully saturated, or fully unsaturated 5- or 6-membered ring, optionally having 1 to 4 heteroatoms independently selected from the group consisting of nitrogen, sulfur, and oxygen;

A¹ for each occurrence is independently optionally substituted, on one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —CONX⁶X⁶, —CO₂X⁶, oxo, C₁-C₆ alkyl, nitro, cyano, benzyl, —SOₘ(C₁-C₆ alkyl), 1H-tetrazol-5-yl, phenyl, phenoxy, phenylalkyloxy, halophenyl, methylenedioxy, —NX⁶X⁶, —NX⁶COX⁶, —SO₂NX⁶X⁶, —NX⁶SO₂-phenyl, NX⁶SO₂X⁶, —CONX¹¹X¹², —SO₂NX¹¹X¹², —NX⁶SO₂X¹², —NX⁶CONX¹¹X¹², —NX⁶SO₂NX¹¹X¹², —NX⁶COX¹², imidazolyl, thiazolyl, and tetrazolyl, provided that if A¹ is optionally substituted with methylenedioxy then it can only be substituted with one methylenedioxy;

wherein X¹¹ is hydrogen or C₁-C₆ alkyl optionally independently substituted with phenyl, phenoxy, C₁-C₆ alkoxycarbonyl, —SOₘ(C₁-C₆ alkyl), 1to 5 halo groups, 1to 3 hydroxy groups, 1to 3 C₁-C₁₀ alkanoyloxy groups, or 1to 3 C₁-C₆ alkoxy groups;

X¹² is hydrogen, C₁-C₆ alkyl, phenyl, thiazolyl, imidazolyl, furyl, or thienyl, provided that when X¹² is not hydrogen, the X¹² group is optionally substituted with one to three substituents independently selected from the group consisting of Cl, F, CH₃, OCH₃, OCF₃, and CF₃;

or X¹¹ and X¹² are taken together to form —(CH₂)ᵣ—L¹—(CH₂)ᵣ—, wherein L¹ is CX²X², O, SOₘ, or NX²;

r for each occurrence is independently 1, 2, or 3;

x² for each occurrence is independently hydrogen, optionally substituted C₁-C₆ alkyl, or optionally substituted C₃-C₇ cycloalkyl, wherein the optionally substituted C₁-C₆ alkyl and optionally substituted C₃-C₇ cycloalkyl in the definition of X² are optionally independently substituted with —SOₘ(C₁-C₆ alkyl), —CO₂X³, 1 to 5 halo groups, or 1-3 OX³ groups;

X³ for each occurrence is independently hydrogen or C₁-C₆ alkyl;

X⁶ for each occurrence is independently hydrogen, optionally substituted C₁-C₆ alkyl, halogenated C₂-C₆ alkyl, optionally substituted C₃-C₇ cycloalkyl, halogenated C₃-C₇ cycloalkyl, wherein the optionally substituted C₁-C₆ alkyl and optionally substituted C₃-C₇ cycloalkyl in the definition of X⁶ are optionally independently mono- or di-substituted with C₁-C₄ alkyl, hydroxy, C₁-C₄ alkoxy, carboxyl, CONH₂, —SOₘ(C₁-C₆ alkyl), carboxylate (C₁-C₄ alkyl) ester, or 1H-tetrazol-5-yl; or

when there are two X⁶ groups on one atom and both X⁶ are independently C₁-C₆ alkyl, the two C₁-C₆ alkyl groups may be optionally joined, and together with the atom to which the two X⁶ groups are attached, form a 4- to 9- membered ring optionally having oxygen, sulfur, or NX⁷ as a ring member, wherein X⁷ is hydrogen or C₁-C₆ alkyl optionally substituted with hydroxy;

m for each occurrence is independently 0, 1, or 2; with the provisos that:

X⁶ and X¹² cannot be hydrogen when attached to CO or SO₂ in the form COX⁶, COX¹², SO₂X⁶ or SO₂X¹²; and

when R⁶ is a bond then L is NX² and each r in the definition —(CH₂)ᵣ—L—(CH₂)ᵣ— is independently 2 or 3.

Another group of preferred growth hormone secretagogues for use in the compositions, methods, and kits of the present invention are those wherein the growth hormone secretagogue is 2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide; 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide; 2-amino-N-{1(R)-benzyloxymethyl-2-[1,3-dioxo-8a(S)-pyridin-2-ylmethyl-2-(2,2,2,-trifluoro-ethyl)-hexahydro-imidazo[1,5-a]pyrazin-7-yl]-2-oxo-ethyl}-2-methyl-propionamide; N-(1(R)-((1,2-dihydro-1-methanesulfonyl-spiro(3H-indole-3,4′-piperidin)-1′-yl)carbonyl)-2-(phenylmethyloxy)ethyl)-2-amino-2-methyl-propanamide; or a prodrug of any of these compounds, or a pharmaceutically acceptable salt of any of said compounds or said prodrugs.

### DETAILED DESCRIPTION OF THE INVENTION

The present invention is directed to pharmaceutical compositions, methods, and kits comprising a CRF antagonist and a growth hormone secretagogue or growth hormone useful for treating a wide variety of diseases and conditions as fully described herein. While many specific compounds that serve as CRF antagonists, growth hormones, or growth hormone secretagogues are described and discussed herein, all such compounds, either cited herein or not, presently known or yet to be discovered, are considered to be useful in the practice of the present invention.

The second component of the compositions, methods, and kits of the present invention is a growth hormone secretagogue or growth hormone per se.

A representative first class of growth hormone secretagogues is set forth in PCT publication WO 97/24369, which is incorporated herein by reference, as compounds having the formula III:

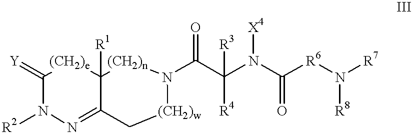

wherein the various substituents are as defined in WO 97/24369. Said compounds are prepared as disclosed therein.

Preferred members of this first class of growth hormone secretagogues are 2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide, having the following structure:

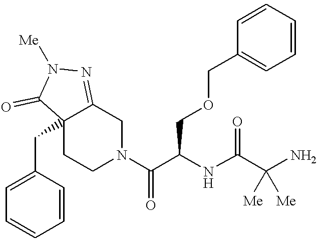

and 2-amino-N-(1-(R)-(2,4-difluoro-benzyloxymethyl)-2-oxo-2-(3-oxo-3a-(R)-pyridin-2ylmethyl)-2-(2,2,2-trifluoro-ethyl)-2,3,a,4,6,7-hexahydro-pyrazolo-[4,3-c]pyfidin-5-yl-ethyl)-2-methyl-propionamide, having the following structure:

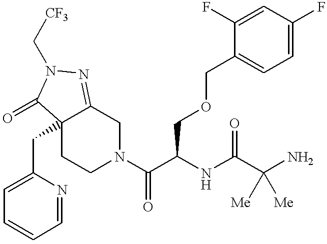

both of which are within the scope of the disclosure of international patent application publication number WO 97/24369. With respect to the latter compound, see also WO 98/58948.

A representative second class of growth hormone secretagogues is set forth in U.S. Pat. No., 5,206,235, which is incorporated herein by reference, as having the following structure:

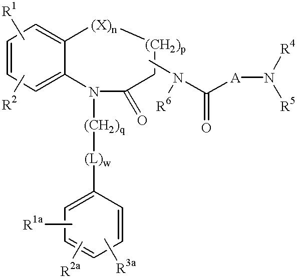

wherein the various substituents are as defined in U.S. Pat. No. 5,206,235. Said compounds are prepared as disclosed therein.

Preferred compounds within this second class include 3-(2(R)-hydroxypropyl)amino-3-methyl-N-[2,3,4,5-tetrahydro-2-oxo-1-[[2′-(1H-tetrazol-5-yl)[1,1′-biphenyl]-4-yl]methyl-1H-1-benzazepin-3(R)-yl]butanamide, having the following structure:

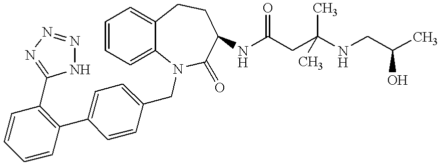

and 3-amino-3-methyl-N-[2,3,4,5-tetrahydro-2-oxo-1-[[2′-(1H-tetrazol-5-yl)[1,1′-biphenyl]-4-yl]methyl-1H-1-benzazepin-3(R)-yl]butanamide, having the following structure:

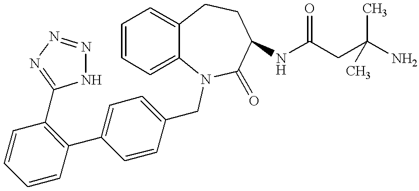

both of which are disclosed in U.S. Pat. No. 5,206,235.

A representative third class of growth hormone secretagogues is set forth in U.S. Pat. No. 5,283,241, which is incorporated herein by reference, as having the following formula:

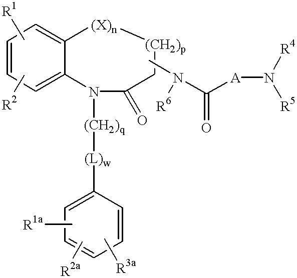

wherein the various substituents are as defined in U.S. Pat. No. 5,283,241. Said compounds are prepared as disclosed therein.

A representative fourth class of growth hormone secretagogues is disclosed in PCT publication WO 97/41879, which is incorporated herein by reference, as compounds having the following formulas:

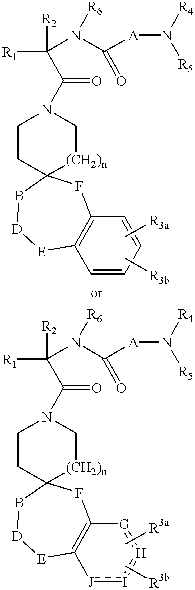

wherein the various substituents are as defined in WO 97/41879. Said compounds are prepared as disclosed therein.

The most preferred compound within this fourth class which may be employed in the present invention is identified as N-[1(R)-[(1,2-dihydro-1-methanesulfonylspiro[3H-indole-3,4′-piperidin]-1′-yl)carbonyl]-2-(phenyl-methyloxy)ethyl]-2-amino-2-methylpropanamide, having the following structure:

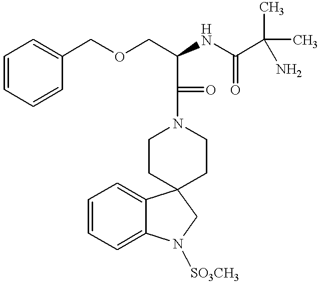

or a pharmaceutically acceptable salt thereof, in particular, the methanesulfonate salt, all of which are disclosed in WO 97/41879.

A representative fifth class of growth hormone secretagogues is disclosed in U.S. Pat. No. 5,492,916, which is incorporated herein by reference, as being compounds of the formula:

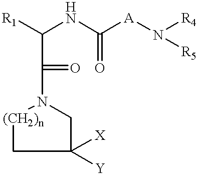

wherein the various substituents are as defined in U.S. Pat. No. 5,492,916. Said compounds are prepared as disclosed therein.

A representative sixth class of growth hormone secretagogues is set forth in WO 98/58947, as compounds having the formula:

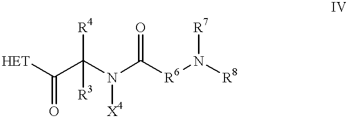

wherein the various substituents are as defined in WO 98/58947. The preparation of the compounds of formula IV of the present invention can be carried out in sequential or convergent synthetic routes. Syntheses detailing the preparation of the compounds of formula IV in a sequential manner are presented in WO 98/58947.

The expression “prodrug” refers to compounds that are drug precursors which, following administration, release the drug in vivo via some chemical or physiological process (e.g., a prodrug on being brought to the physiological pH is converted to the desired drug form). A prodrug of any or all of the compounds (i.e., a CRF antagonist, a growth hormone secretagogue, or a growth hormone) may be used in the methods, kits, and compositions of the instant invention. Upon cleavage, exemplary prodrugs release the corresponding free acid (where applicable), and such hydrolyzable ester-forming residues of the prodrugs of this invention include but are not limited to carboxylic acid substituents wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl, piperidino-, pyrrolidino-, or morpholino(C₂-C₃)alkyl, and the like.

Other exemplary prodrugs (where applicable) are derivatives of an alcohol of the compounds used in this invention wherein the free hydrogen of a hydroxyl substituent is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxy-carbonylamino-methyl, succinoyl, (C₁-C₆)alkanoyl, α-amino(C₁-C₄)alkanoyl, arylacetyl, α-aminoacyl, α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂, glycosyl (the radical resulting from detachment of the hydroxyl of the hemiacetal of a carbohydrate), or the like.

Of the compositions, methods, and kits of the present invention as defined and claimed herein, particularly preferred are those compositions, methods, and kits that contain one of the following two CRF antagonists:

4-(1-ethyl-propoxy)-3,6-dimethyl-2-(2,4,6-trimethylphenoxy)-pyridine, having the formula

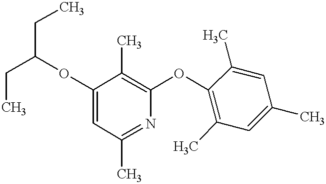

or (3,6-dimethyl-2-(2,4,6-trimethyl-phenoxy)-pyridin-4-yl)-(1-ethyl-propyl)-amine, having the formula

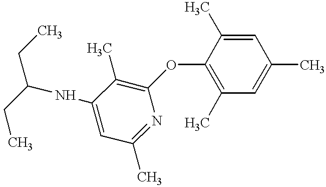

and alternatively one of the following two growth hormone secretagogues: 2-amino-N-[2-(3a(R)-benzyl-2-methyl-3-oxo-2,3,3a,4,6,7-hexahydro-pyrazolo-[4,3-c]pyridin-5-yl)-1(R)-benzyloxymethyl-2-oxo-ethyl]-isobutyramide, having the formula:

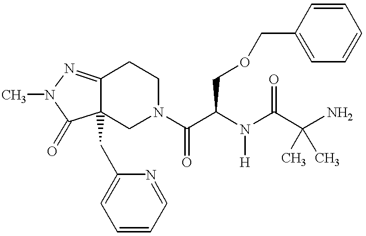

or 2-amino-N-(1(R)-(2,4-difluoro-benzyloxymethyl)-2-oxo-2-(3-oxo-3a(R)-(pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide, having the formula:

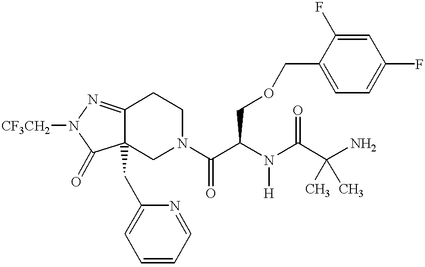

In the preferred kits of the present invention, the pharmaceutical composition comprising a CRF antagonist is a pharmaceutical composition comprising one of the preferred CRF antagonists as defined above (i.e., 4-(1-ethyl-propoxy)-3,6-dimethyl-2-(2,4,6-trimethylphenoxy)-pyridine or (3,6-dimethyl-2-(2,4,6-trimethyl-phenoxy)-pyridin-4-yl)-(1-ethyl-propyl)-amine).

In the preferred kits of the present invention, the pharmaceutical composition comprising a growth hormone secretagogue is a pharmaceutical composition comprising one of the preferred growth hormone secretagogues as defined above (i.e., 2-amino-N-[2-(3a(R)-benzyl-2-methyl-3-oxo-2,3,3a,4,6,7-hexahydro-pyrazolo-[4,3-c]pyridin-5-yl)-1(R)-benzyloxymethyl-2-oxo-ethyl]-isobutyramide or 2-amino-N-(1(R)-(2,4-difluoro-benzyloxymethyl)-2-oxo-2-(3-oxo-3a(R)-(pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide).

In the preferred kits of the present invention comprising both a pharmaceutical composition comprising a CRF antagonist and a pharmaceutical composition comprising a growth hormone secretagogue, the pharmaceutical composition comprising a CRF antagonist comprises a preferred CRF antagonist as defined above and the pharmaceutical composition comprising a growth hormone secretagogue comprises a preferred growth hormone secretagogue as defined herein.

The preferred methods of treatment of the present invention are those methods that employ a preferred CRF antagonist, growth hormone secretagogue, or a pharmaceutical composition(s) of the present invention, as defined herein.

Also preferred are those methods that employ a preferred CRF antagonist, growth hormone secretagogue, or a pharmaceutical composition(s) of the present invention, as defined herein, for treating or preventing osteoporosis or frailty associated with aging or obesity, cardiovascular or heart related disease, in particular hypertension, tachycardia, and congestive heart failure, accelerating bone fracture repair, attenuating protein catabolic response after a major operation, reducing cachexia and protein loss due to chronic illness, accelerating wound healing, or accelerating the recovery of burn patients or of patients having undergone major surgery.

Presently most preferred, the pharmaceutical compositions, methods, and kits of the present invention can be used for treating and preventing congestive heart failure.

Preferably, the combinations of pharmaceutically active compounds of the present invention show a synergistic effect and/or show less side effects, as compared to the individual compounds, when treating a mammal, preferably a human. Thus, in treating or preventing a particular disease, at a specific dosage level, the combinations of the present invention show a better activity than the activity which could be expected when administering the individual compounds and/or show less (or less severe) side effects than could be expected when administering the individual compounds.

The compositions and combinations of this invention can be administered by oral, parenteral (e.g., intramuscular, intraperitoneal, intravenous, or subcutaneous injection, or through an implant), nasal, vaginal, rectal, sublingual, or topical routes of administration and can be formulated with pharmaceutically acceptable carriers, vehicles, or diluents to provide dosage forms appropriate for each route of administration.

Solid dosage forms for oral administration include capsules, tablets, pills, powders, granules, and the like, and for non-human mammals (cats and dogs are the presently preferred non-human mammals) the solid dosage forms can include admixtures with food and chewable forms. In such solid dosage forms, the compounds and combinations of this invention can be admixed with at least one inert pharmaceutically acceptable carrier such as sucrose, lactose, starch, or the like. Such dosage forms can also comprise, as is normal practice, additional substances other than such inert diluents, e.g., lubricating agents such as magnesium stearate. In the case of capsules, tablets, and pills, the dosage forms may also comprise buffering agents. Tablets and pills can additionally be prepared with enteric coatings. In the case of chewable forms, the dosage form may comprise flavoring agents and perfuming agents.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants (such as wetting agents), emulsifying and suspending agents, sweetening agents, flavorings, perfuming agents, and the like.

Preparations according to this invention for parenteral administration include sterile aqueous or non-aqueous solutions, suspensions, emulsions, and the like. Examples of non-aqueous solvents or vehicles are propylene glycol, polyethylene glycol, vegetable oils such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Compositions for rectal or vaginal administration are preferably suppositories that may contain, in addition to the active substance, excipients such as cocoa butter or a suppository wax.

Compositions for nasal or sublingual administration are also prepared with standard excipients well known in the art.

The dosage of active ingredients in the compositions and methods of this invention may be varied; however, it is necessary that the amount of the active ingredients in such compositions be such that a suitable dosage form is obtained. The selected dosage depends upon the desired therapeutic effect, on the route of administration, the particular compounds administered, the duration of the treatment, and other factors. All dosage ranges and dosage levels mentioned herein refer to each pharmaceutically active compound present in the pharmaceutical compositions and kits of the present invention, as well as those used in the methods of the present invention. Generally, dosage levels of between 0.0001 to 100 mg/kg of body weight daily are administered to humans and other animals, e.g., mammals.

A preferred dosage range in humans is 0.01 to 5.0 mg/kg of body weight daily which can be administered as a single dose or divided into multiple doses.

A preferred dosage range in mammals other than humans is 0.01 to 10.0 mg/kg of body weight daily which can be administered as a single dose or divided into multiple doses. A more preferred dosage range in mammals other than humans is 0.1 to 5.0 mg/kg of body weight daily which can be administered as a single dose or divided into multiple doses.

The present invention includes within its scope the use of a combination of this invention, e.g., a corticotropin releasing factor antagonist and a growth hormone secretagogue or growth hormone, for the prevention or treatment of congestive heart failure in mammals. The preferred mammal for purposes of this invention is a human.

Since the present invention has an aspect that relates to treatment with a combination of active ingredients which may be administered separately, the invention also relates to combining separate pharmaceutical compositions in kit form. Thus, in one embodiment, the kit comprises two separate pharmaceutical compositions: a corticotropin releasing factor antagonist, a prodrug thereof, or a pharmaceutically acceptable salt of said corticotropin releasing factor antagonist or said prodrug; and a growth hormone secretagogue, a prodrug thereof, or a pharmaceutically acceptable salt of said growth hormone secretagogue or said prodrug. The kit also comprises a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil that is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several tablets or capsules to be taken on a given day. Also, a daily dose of a corticotropin releasing factor antagonist, a prodrug thereof, or a pharmaceutically acceptable salt of said corticotropin releasing factor antagonist or said prodrug can consist of one tablet or capsule, while a daily dose of the growth hormone secretagogue, prodrug thereof, or pharmaceutically acceptable salt of said growth hormone secretagogue or said prodrug can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment, the present invention comprises kits comprising a pharmaceutical composition, a package, and a package insert. The pharmaceutical composition of these kits contains either a corticotropin releasing factor antagonist or a growth hormone/growth hormone secretagogue. The kits of the present invention containing a pharmaceutical composition containing a corticotropin releasing factor antagonist differ from known kits containing a pharmaceutical composition containing a corticotropin releasing factor antagonist in that on the package and/or on the package insert of the kits it is stated that the pharmaceutical composition is to be administered together with a pharmaceutical composition containing a growth hormone or growth hormone secretagogue. The kits of the present invention containing a pharmaceutical composition containing a growth hormone or growth hormone secretagogue differ from known kits containing a pharmaceutical composition containing a growth hormone or growth hormone secretagogue in that on the package and/or on the package insert of the kits it is stated that the pharmaceutical composition is to be administered together with a pharmaceutical composition containing a corticotropin releasing factor antagonist.

The term “together with” as used in the immediately preceding paragraph is intended to encompass the simultaneous administration of the two pharmaceutical compositions (e.g., a tablet containing one pharmaceutical composition is to be administered orally while the other pharmaceutical composition is administered by way of infusion, two tablets or capsules are to be swallowed together, etc.). The term “together with” is also intended to include the administration of the two pharmaceutical compositions in a specifically timed manner, i.e., one pharmaceutical composition is to be administered a certain time period after administration of the other pharmaceutical composition. The time period in which the two pharmaceutical compositions are to be administered must be sufficiently short for the corticotropin releasing factor antagonist and the growth hormone secretagogue to exhibit their activity contemporaneously, preferably in a synergistic manner. The exact time period depends on the specific compounds of the pharmaceutical compositions, the application route, the kind and severeness of the disease to be treated, the kind, age, and condition of the patient to be treated, etc., and can be determined by a physician using known methods in combination with the disclosure of the present invention. Generally, the two compositions are to be administered within one day, preferably within 5 hours, more preferably within 2 hours, and even more preferably within one hour. Most preferably, the two compositions are to be administered at the same time or one immediately after the other.

Methods that may be used to determine CRF antagonist activity of the compounds employed to practice the present invention are as described in, e.g., Wynn et al., Endocrinology, 116:1653-59 (1985), and Grigoriadis et al., Peptides, 10:179-88 (1989). Methods that can be used to determine the CRF binding protein inhibiting activity of compounds employed to practice the present invention are described in Smith et al., Brain Research, 745(1,2):248-56 (1997). These methods determine the binding affinity of a test compound for a CRF receptor, which is highly related to its expected activity as a CRF antagonist.

The combinations of this invention, i.e., a corticotropin releasing factor antagonist and growth hormone or a growth hormone secretagogue, may be tested for hypoglycemic activity according to the following procedure.

Five to eight week old C₅₇ BL/6J-ob/ob mice (obtained from Jackson Laboratory, Bar Harbor, Me.) are housed five per cage under standard animal care practices. After a one week acclimation period, the animals are weighed and 25 microliters of blood are collected via an ocular bleed prior to any treatment. The blood sample is immediately diluted 1:5 with saline containing 2% sodium heparin, and held on ice for glucose analysis. Animals are then regrouped, in groups of five per cage, such that the mean glucose values of the groups are similar, dosed daily for five days with test compounds (0.01-100 mg/kg), a positive control such as englitazone or ciglitazone (50 mg/kg p.o.), (U.S. Pat. No. 4,467,902; Sohda et al., Chem. Pharm. Bull., 32:4460-65 (1984)), or vehicle. All compounds are administered by oral gavage in a vehicle consisting of 0.25% w/v methyl cellulose. On day 5, the animals are weighed again and bled (via the ocular route) for blood glucose level determinations. The freshly collected samples are centrifuged for two minutes at 10,000×g at room temperature. The supernatant is analyzed for glucose, for example, by the ABA 200 Bichromatic Analyzer™¹, using the A-gent™ glucose UV reagent system² (hexokinase method) using 20, 60 and 100 mg/dl standards. Plasma glucose is then calculated by the equation,

Plasma glucose (mg/dl)=Sample value×5×1.67=Sample value×8.35, where 5 is the dilution factor and 1.67 is the plasma hematocrit adjustment (assuming the hematocrit is 40%). ¹ ™A registered trademark of Abbott Laboratories, Diagnostics Division, 820 Mission Street, So. Pasadena, Calif. 91030. ² A modification of the method of Richterrich et al., Schweizerische Medizinische Wochenschrift, 101:860 (1971).

The animals dosed with vehicle maintain substantially unchanged hyperglycemic glucose levels (e.g., 250 mg/dl), while positive control animals have depressed glucose levels (e.g., 130 mg/dl). The glucose lowering activity of test compounds is expressed in terms of % glucose normalization. For example, a glucose level that is the same as the positive control is expressed as 100%.

The invention that is claimed is:

1. A pharmaceutical composition comprising a corticotropin releasing factor antagonist and a growth hormone secretagogue or growth hormone.

2. A pharmaceutical composition according to claim 1 wherein said corticotropin releasing factor antagonist is a compound of formula 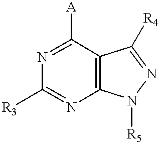
or a pharmaceutically acceptable acid addition salt thereof, wherein
A is NR₁R₂, CR₁R₂R₁₁, or C(═CR₁R₁₂)R₂, NHCR₁R₂R₁₁, OCR₁R₂R₁₁, SCR₁R₂R₁₁, NHNR₁R₂, CR₂R₁₁NHR₁, CR₂R₁₁OR₁, CR₂R₁₁SR₁ or C(O)R₂;
R₁is hydrogen, or C₁-C₆ alkyl which may be substituted by one or two substituents R₆ independently selected from the group consisting of hydroxy, fluoro, chloro, bromo, iodo, C₁-C₆ alkoxy, O—C(O)—(C₁-C₆ alkyl), O—C(O)—N(C₁-C₄ alkyl)(C₁-C₂ alkyl); amino, NH(C₁-C₄ alkyl), S(C₁-C₆ alkyl), OC(O)NH(C₁-C₄ alkyl), N(C₁-C₂ alkyl)C(O)(C₁-C₄ alkyl), NHC(O)(C₁-C₄ alkyl), COOH, CO(C₁-C₄ alkyl), C(O)NH(C₁-C₄ alkyl), C(O)N(C₁-C₄ alkyl)(C₁-C₂ alkyl), SH, CN, N0₂, SO(C₁-C₄ alkyl); SO₂(C₁-C₄ alkyl), SO₂NH(C₁-C₄ alkyl), SO₂N(C₁-C₄ alkyl)(C₁-C₂ alkyl), and said C₁-C₆ alkyl may have one or two double or triple bonds;
R₂ is C₁-C₁₂ alkyl, aryl or (C₁-C₁₀ alkylene)aryl wherein said aryl is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, azaindolyl, oxazolyl, or benzoxazolyl; 3- to 8-membered cycloalkyl or (C₁-C₆ alkylene) cycloalkyl, wherein said cycloalkyl may have one or two of O, S or N—Z, wherein Z is hydrogen, substituted , independently, for one or two carbons of said cycloalkyl, C₁-C₄ alkyl, benzyl or C₁-C₄ alkanoyl, wherein R² may be substituted independently by from one to three of chloro, fluoro, or C₁-C₄ alkyl, or one of hydroxy, bromo, iodo, C₁-C₆ alkoxy, OC(O)(C₁-C₆ alkyl), O—C—N(C₁-C₄ alkyl)(C₁-C₂ alkyl), S(C₁-C₆ alkyl), NH₂, NH(C₁-C₂ alkyl), N(C₁-C₄ alkyl) C(O)(C₁-C₄ alkyl), NHC(O)(C₁-C₄ alkyl), COOH, C(O)O(C₁-C₄ alkyl), C(O)NH(C₁-C₄ alkyl), C(O)N(C₁-C₄ alkyl)(C₁-C₂ alkyl), SH, CN, NO₂, SO(C_{1-C}₄ alkyl), SO₂(C₁-C₄ alkyl), SO₂NH(C₁-C₄ alkyl), SO₂N(C₁-C₄ alkyl)(C₁-C₂ alkyl), and wherein said C₁-C₁₂ alkyl or C₁-C₁₀ alkylene may have one to three double or triple bonds; or
NR₁R₂ or CR₁R₂R₁₁ may form a 4- to 8-membered ring optionally having one or two double bonds or one or two of O, S or N—Z wherein Z is hydrogen, C₁-C₄ alkyl, benzyl, or C₁-C₄ alkanoyl;
R₃ is hydrogen, C₁-C₆ alkyl, fluoro, chloro, bromo, iodo, hydroxy, amino, O(C₁-C₆ alkyl), NH(C₁-C₆ alkyl), N(C₁-C₄ alkyl)(C₁-C₂ alkyl), SH, S(C₁-C₄ alkyl), SO(C₁-C₄ alkyl), or SO₂(C₁-C₄ alkyl), wherein said C₁-C₄ alkyl and C₁-C₆ alkyl may have one or two double or triple bonds and may be substituted by from 1 to 3 R₇ substituents independently selected from the group consisting of hydroxy, amino, C₁-C₃ alkoxy, dimethylamino, diethylamino, methylamino, ethylamino, NHC(O)CH₃, fluoro, chloro or C₁-C₃ thioalkyl;
R₄ is hydrogen, C₁-C₆ alkyl, fluoro, chloro, bromo, iodo, C₁-C₆ alkoxy, amino, NH(C₁-C₆ alkyl), N(C₁-C₆ alkyl) (C₁-C₂ alkyl), SOₙ(C₁-C₆ alkyl), wherein n is 0, 1 or 2, cyano, hydroxy, carboxy, or amido, wherein said C₁-C₆ alkyls may be substituted by one to three of hydroxy, amino, carboxy, amido, NHC(O)(C₁-C₄ alkyl), NH(C₁-C₄ alkyl), N(C₁-C₄ alkyl)(C₁-C₂ alkyl), C(O)O(C₁-C₄ alkyl), C₁-C₃ alkoxy, C₁-C₃ thioalkyl, fluoro, bromo, chloro, iodo, cyano or nitro;
R₅ is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzoisothiazolyl, thiazolyl, isoxazolyl, benzisoxazolyl, benzimidazolyl, triazolyl, pyrazolyl, pyrrolyl, indolyl, pyrrolopyridyl benzoxazolyl, oxazolyl, pyrrolidinyl, thiazolidinyl, piperazinyl, piperidinyl, or tetrazolyl, wherein each one of the above groups may be substituted independently by from one to three of fluoro, chloro, bromo, formyl, C₁-C₆ alkyl, C₁-C₆ alkoxy or trifluoromethyl, or one of hydroxy, iodo, cyano, nitro, amino, cyclopropyl, NH(C₁-C₄ alkyl), N(C₁-C₄ alkyl)(C₁-C₂ alkyl), COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), SO₂(C₁-C₆ alkyl), wherein said C₁-C₄ alkyl and C₁-C₆ alkyl may have one double or triple bond and may be substituted by one or two of fluoro, chloro, hydroxy, amino, methylamino, dimethylamino or acetyl; with the proviso that R₅ is not unsubstituted phenyl;
R₁₁is hydrogen, hydroxy, fluoro, chloro, COO(C₁-C₂ alkyl), cyano, or CO(C₁-C₂ alkyl); and
R₁₂ is hydrogen or C₁-C₄ alkyl; with the provisos that:
(a) A is not straight chain C₁-C₁₂ alkyl;
(b) when R₃ is hydrogen, A is benzyl or phenethyl, and R₄ is fluoro, chloro, bromo or iodo, then R₅ is not 5′-deoxy-ribofuranosyl or 5′-amino-5′-deoxy-ribofuranosyl; and
(c) when R⁵ is phenyl, said phenyl is substituted by two or three substituents.

3. A pharmaceutical composition according to claim 1 wherein said corticotropin releasing factor antagonist is a compound of formula 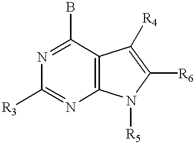
and the pharmaceutically acceptable acid addition salts thereof, wherein
B is NR₁R₂, CR₁R₂R₁₁, C(═CR₂R₁₂)R₁, NHR₁R₂R₁₁, OCR₁R₂R₁₁, SCR₁R₂R₁₁, NHNR₁R₂, CR₂R₁₁NHR, CR₂R₁₁OR₁, CR₂R₁₁SR₁, or C(O)R₂;
R₁ is hydrogen, or C₁-C₆ alkyl which may be substituted by one or two substituents R₇ independently selected from the group consisting of hydroxy, fluoro, chloro, bromo, iodo, C₁-C₈ alkoxy, O—C(═O)—(C₁-C₆ alkyl), O—C(═O)NH(C₁-C₄ alkyl), O—C(═O)—N(C₁-C₄ alkyl)(C₁-C₂ alkyl), amino, NH(C₁-C₄ alkyl), N(C₁-C₂ alkyl)(C₁-C₄ alkyl), S(C₁-C₆ alkyl), N(C₁-C₄ alkyl)C(═O)(C₁-C₄ alkyl), NH(C₁-C₄ alkyl), COOH, C(═O)O(C₁-C₄ alkyl), C(═O)NH(C₁-C₄ alkyl), C(═O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂ alkyl), and said C₁-C₆ alkyl may contain one or two double or triple bonds;
R₂ is C₁-C₁₂ alkyl, aryl or (C₁-C₁₀ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, pyrrolopyridyl, oxazolyl, or benzoxazolyl; 3- to 8-membered cycloalkyl or (C₁-C₆ alkylene) cycloalkyl, wherein said cycloalkyl may contain one or two of O, S or N—Z wherein Z is hydrogen, C₁-C₄ alkyl, benzyl or C₁-C₄ alkanoyl, wherein R₂ may be substituted independently by from one to three of chloro, fluoro, or C₁-C₄ alkyl, or one of hydroxy, bromo, iodo, C₁-C₆ alkoxy, O—C(═O)—(C₁-C₆ alkyl), O—C—N(C₁-C₄ alkyl)(C₁-C₂ alkyl), S(C₁-C₆ alkyl), NH₂, NH(C₁-C₂ alkyl), N(C₁-C₂ alkyl) (C₁-C₄ alkyl), N(C₁-C₄ )—C(═O)(C₁-C₄ alkyl), NHC(═O)(C₁-C₄), COOH, C(═O)O(C₁-C₄ alkyl), C(═O)NH(C₁-C₄ alkyl), C(═O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂ alkyl), and wherein said C₁-C₁₂ alkyl or C₁-C₁₀ alkylene may contain one to three double or triple bonds; or
NR₁R₂ or CR₁R₂R₁₁ may form a saturated 3- to 8 membered carbocyclic ring of which the 5- to 8-membered ring contain one or two double bonds or one or two of O, S or N—Z wherein Z is hydrogen, C₁-C₄ alkyl, benzyl or C₁-C₄ alkanoyl;
R₃ is hydrogen, C₁-C₆ alkyl, fluoro, chloro, bromo, iodo, hydroxy, amino, O(C₁-C₆ alkyl), NH(C₁-C₆ alkyl), N(C₁-C₄ alkyl)(C₁-C₂ alkyl), SH, S(C₁-C₄ alkyl), SO(C₁-C₄ alkyl), or SO₂(C₁-C₄ alkyl), wherein said C₁-C₄ alkyl and C₁-C₆ alkyl may contain from one or two double or triple bonds and may be substituted by from 1 to 3 substituents R₈ independently selected from the group consisting of hydroxy, amino, C₁-C₃ alkoxy, dimethylamino, diethylamino, methylamino, ethylamino, NHCH₃, fluoro, chloro or C₁-C₃ thioalkyl;
R₄ and R₆ are each independently hydrogen, C₁-C₆ alkyl, fluoro, chloro, bromo, iodo, C₁-C₆ alkoxy, amino, NH(C₁-C₆ alkyl), N(C₁-C₆ alkyl)(C₁-C₂ alkyl), SOₙ(C₁-C₆ alkyl), wherein n is 0, 1 or 2, cyano, hydroxy, carboxy, or amido, wherein said C₁-C₆ alkyls may be substituted by one to three of hydroxy, amino, carboxy, amido, NHC(═O)(C₁-C₄ alkyl), NH(C₁-C₄ alkyl), N(C₁-C₄ alkyl)(C₁-C₂ alkyl), C(═O)O(C₁-C₄ alkyl), C₁-C₃ alkoxy, C₁-C₃ thioalkyl, fluoro, bromo, chloro, iodo, cyano or nitro;
R₅ is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, azaindolyl, benzoxazolyl, oxazolyl, pyrrolidinyl, thiazolidinyl, morpholinyl, piperidinyl, piperazinyl, tetrazolyl, or 3- to 8-membered cycloalkyl or 9- to 12-membered bicycloalkyl, optionally containing one to three of O, S or N—Z wherein Z is hydrogen, C₁-C₄ alkyl, C₁-C₄ alkanoyl, phenyl or phenylmethyl, wherein each one of the above groups may be substituted independently by from one to four of fluoro, chloro, C₁-C₆ alkyl, C₁-C₆ alkoxy or trifluoromethyl, or one of bromo, iodo, cyano, nitro, amino, NH(C₁-C₄ alkyl), N(C₁-C₄)(C₁-C₂ alkyl), COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), SO₂(C₁-C₆ alkyl), wherein said C₁-C₄ alkyl and C₁-C₆ alkyl may be substituted by one or two of fluoro, chloro, hydroxy, amino, methylamino, dimethylamino or acetyl; with the proviso that R₅ is not unsubstituted phenyl;
R₁₁is hydrogen, hydroxy, fluoro, chloro, COO(C₁-C₂ alkyl), cyano, or CO(C₁-C₂ alkyl); and
R₁₂ is hydrogen or C₁-C₄ alkyl; with the proviso that (1) when R₅ is 4-bromophenyl, R₃ is hydrogen, and R₄ and R₆ are methyl, then B is not methylamino or ethyl, and (2) when R₅ is 4-bromophenyl, and R₃, R₄ and R₆ are methyl, then B is not 2-hydroxyethylamino.

4. A pharmaceutical composition according to claim 1 wherein said corticotropin releasing factor antagonist is a compound of formula 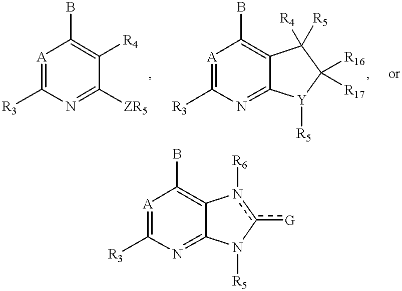
wherein
A is CR₇ or N;
B is NR₁R₂, CR₁R₂R₁₁, C(═CR₂R₁₂)R₁, NHCHR₁R₂, OCHR₁R₂, SCHR₁R₂, CHR₂OR₁₂, CHR₂SR₁₂, C(S)R₂ or C(O)R₂;
G is oxygen, sulfur, NH, NH₃, hydrogen, methoxy, ethoxy, trifluoromethoxy, methyl, ethyl, thiomethoxy, NH₂, NHCH₃, N(CH₃)₂ or trifluromethyl;
Y is CH or N;
Z is NH, O, S, N (C₁-C₂ alkyl), or CR₁₃R₁₄, wherein R₁₃ and R₁₄ are each independently hydrogen, trifluoromethyl, or C₁-C₄ alkyl, or one of R₁₃ and R₁₄ may be cyano, chloro, bromo, iodo, fluoro, hydroxy, O(C₁-C₂ alkyl), amino, NH(C₁-C₂ alkyl), or CR₁₃R₁₄ may be C═O or cyclopropyl;
R₁is C₁-C₆ alkyl which may be substituted by one or two substituents R₈ independently selected from the group consisting of hydroxy, fluoro, chloro, bromo, iodo, C₁-C₄ alkoxy, O—CO—(C₁-C₄ alkyl), O—CO—NH(C₁-C₄ alkyl), O—CO—N(C₁-C₄ alkyl)(C₁-C₂ alkyl), NH(C₁-C₄ alkyl), N(C₁-C₂ alkyl)(C₁-C₄ alkyl), S(C₁-C₄ alky), N(C₁-C₄alkyl)CO(C₁-C₄ alkyl), NHCO(C₁-C₄ alkyl), COO(C₁-C₄ alkyl), CONH(C₁-C₄ alkyl), CON(C₁-C₄ alkyl)(C₁-C₂ alkyl), S(C₁-C₄ alkyl), CN, NO₂, SO(C₁-C₄ alkyl), SO₂(C₁-C₄ alkyl), and said C₁-C₆ alkyl or C₁-C₄ alkyl may contain one double or triple bond;
R₂ is C₁-C₁₂ alkyl, aryl or (C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, or benzoxazolyl; 3- to 8-membered cycloalkyl or (C₁-C₆ alkylene)cycloalkyl, wherein said cycloalkyl may contain one or two of O, S or N—R₉ wherein R₉ is hydrogen, or C₁-C₄ alkyl, wherein the above defined R₂ may be substituted independently by from one to three of chloro, fluoro, or C₁-C₄ alkyl, or one of bromo, iodo, C₁-C₆ alkoxy, O—CO—(C₁-C₆ alkyl), O—CO—N(C₁-C₄ alkyl)(C₁-C₂ alkyl), S(C₁-C₆ alkyl), CN, NO₂, SO(C₁-C₄ alkyl), or SO₂(C₁-C₄ alkyl), and wherein said C₁-C₁₂ alkyl or C₁-C₄ alkylene may contain one double or triple bond; or
NR₁R₂ or CR₁R₂R₁₁ may form a saturated 5- to 8-membered carbocyclic ring which may contain one or two double bonds or one or two of O or S;
R₃ is methyl, ethyl, fluoro, chloro, bromo, iodo, cyano, methoxy, OCF₃, methylthio, methylsulfonyl, CH₂OH or CH₂OCH₃;
R₄ is hydrogen, C₁-C₄ alkyl, fluoro, chloro, bromo, iodo, C₁-C₄ alkoxy, amino, nitro, NH(C₁-C₄ alkyl), N(C₁-C₄ alkyl)(C₁-C₂ alkyl), SOₙ(C₁-C₄ alkyl), wherein n is 0, 1 or 2, cyano, hydroxy, CO(C₁-C₄ alkyl), CHO, or COO(C₁-C₄ alkyl), wherein said C₁-C₄ alkyl may contain one or two double or triple bonds and may be substituted by one or two of hydroxy, amino, carboxy, NHCOCH₃, NH(C₁-C₂ alkyl), N(C₁-C₂ alkyl)₂, COO(C₁-C₄ alkyl), CO(C₁-C₄ alkyl), C₁-C₃ alkoxy, C₁-C₃ thioalkyl, fluoro, chloro, cyano or nitro;
R₅ is phenyl, naphthyl, thienyl, benzothienyl, pyridyl, quinolyl, pyrazinyl, pyrimidyl, furanyl, benzofuranyl, benzothiazolyl, or indolyl, wherein each one of the above groups R₅ is substituted independently by from one to three of fluoro, chloro, C₁-C₆ alkyl, or C₁-C₆ alkoxy, or one of hydroxy, iodo, bromo, formyl, cyano, nitro, trifluoromethyl, amino, NH(C₁-C₄ alkyl), N(C₁-C₆)(C₁-C₂ alkyl), COOH, COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), or SO₂(C₁-C₆ alkyl), wherein said C₁-C₄ alkyl and C₁-C₆ alkyl may be substituted by one or two of fluoro, hydroxy, amino, methylamino, dimethylamino or acetyl;
R₆ is hydrogen, or C₁-C₆ alkyl, wherein said C₁-C₆ alkyl may be substituted by one hydroxy, methoxy, ethoxy or fluoro;
R₇ is hydrogen, C₁-C₄ alkyl, fluoro, chloro, bromo, iodo, cyano, hydroxy, O(C₁-C₄ alkyl), C(O)(C₁-C₄ alkyl), or C(O)O(C₁-C₄ alkyl), wherein the C₁-C₄ alkyl groups may be substituted with one hydroxy, chloro or bromo, or one to three fluoro;
R₁₁is hydrogen, hydroxy, fluoro, or methoxy;
R₁₂ is hydrogen or C₁-C₄ alkyl; and
R₁₆ and R₁₇ are each independently hydrogen, hydroxy, methyl, ethyl, methoxy, or ethoxy, except that they are not both methoxy or ethoxy, and CR₄R₆ and CR₁₆R₁₇ each independently may be C═O.

5. A pharmaceutical composition according to claim 1 wherein said corticotropin releasing factor antagonist is a compound of formula 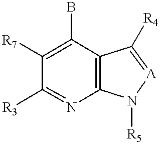
and the pharmaceutically acceptable acid addition salts thereof, wherein
A is N or —CR₆;
B is —NR₁R₂, —CR₁R₂R₁₁, —C(═CR₂R₁₂)R₁, —NHCHR₁R₂, —OCHR₁R₂, —SCHR₁R₂, —CHR₂OR₁₂, —CHR₂SR₁₂, —C(S)R₁ or —C(O)R₁;
R₁ is C₁-C₆ alkyl which may optionally be substituted with one or two substituents independently selected from the group consisting of hydroxy, fluoro, chloro, bromo, iodo, C₁-C₄ alkoxy, —O—CO—(C₁-C₄ alkyl), —O—CO—NH(C₁-C₄ alkyl), —O—CO—N(C₁-C₄ alkyl)(C₁-C₂ alkyl), —NH(C₁-C₄ alkyl), —N(C₁-C₂ alkyl)(C₁-C₄ alkyl), —S(C₁-C₄ alkyl), —N(C₁-C₄alkyl)CO(C₁-C₄ alkyl), —NHCO(C₁-C₄ alkyl), —COO(C₁-C₄ alkyl), —CONH(C₁-C₄ alkyl), —CON(C₁-C₄ alkyl)(C₁-C₂ alkyl), CN, NO₂, —SO(C₁-C₄ alkyl), —SO₂(C₁-C₄ alkyl), and wherein any of the foregoing C₁-C₄ alkyl and C₁-C₆ alkyl groups may optionally contain one carbon-carbon double or triple bond;
R₂ is C₁-C₁₂ alkyl, aryl, —(C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, oxazolyl, or benzoxazolyl; or 3- to 8- membered cycloalkyl or —(C₁-C₆ alkylene)cycloalkyl, wherein one or two of the ring carbons of said cycloalkyl having at least 4 ring members and the cycloalkyl moiety of said —(C₁-C₆ alkylene)cycloalkyl having at least 4 ring members may optionally be replaced by an oxygen or sulfur atom or by N—Z wherein Z is hydrogen; or C₁-C₄ alkyl, and wherein each of said groups R₂ may optionally be substituted with from one to three substituents independently selected from chloro, fluoro, and C₁-C₄ alkyl, or by one substituent selected from bromo, iodo, C₁-C₆ alkoxy, —O—CO—(C₁-C₆ alkyl), —S(C₁-C₆ alkyl), —COO(C₁-C₄ alkyl), CN, NO₂, —SO(C₁-C₄ alkyl), and —SO₂(C₁-C₄ alkyl), and wherein said C₁-C₁₂ alkyl and the C₁-C₄ alkylene moiety of said —(C₁-C₄ alkylene)aryl may optionally contain one carbon-carbon double or triple bond;
or —NR₁R₂ may form a saturated 5- to 8-membered heterocyclic ring, or —CHR₁R₂ may form a saturated 5- to 8-membered carbocyclic ring, wherein each of these rings may optionally contain one or two carbon-carbon double bonds and wherein one or two of the carbon atoms of each of these rings may optionally be replaced with a sulfur or oxygen atom;
R₃ is C₁-C₄ alkyl, fluoro, chloro, bromo, iodo, —CH₂OH, —CH₂OCH₃, —O(C₁-C₃ alkyl), —S(C₁-C₃ alkyl), or —SO₂(C₁-C₃ alkyl), wherein said C₁-C₃ alkyl may optionally contain one carbon-carbon double or triple bond;
R₄ is hydrogen, C₁-C₆ alkyl, fluoro, chloro, bromo, iodo, C₁-C₄ alkoxy, amino, —NHCH₃, —N(CH₃)₂, —CH₂OH, —CH₂OCH₃, or —SOₙ(C₁-C₄ alkyl), wherein n is 0, 1 or 2, cyano, hydroxy, —CO(C₁-C₄ alkyl), —CHO, or —COO(C₁-C₄ alkyl) wherein the C₁-C₄ alkyl moieties in the foregoing R₄ groups may optionally contain one carbon-carbon double or triple bond;
R₅ is phenyl, naphthyl, thienyl, benzothienyl, pyridyl, pyrimidyl, benzofuranyl, pyrazinyl or benzothiazolyl, wherein each one of said groups R₅ may optionally be substituted with from one to three substituents independently selected from fluoro, chloro, C₁-C₆ alkyl and C₁-C₆ alkoxy, or by one substituent selected from iodo, hydroxy, bromo, formyl, cyano, nitro, amino, trifluoromethyl, —NH(C₁-C₄ alkyl), —N(C₁-C₆)(C₁-C₂ alkyl), —COO(C₁-C₄ alkyl), —CO(C₁-C₄ alkyl), —COOH, —SO₂NH(C₁-C₄ alkyl), —SO₂N(C₁-C₄ alkyl)(C₁-C₂ alkyl), —SO₂NH₂, —NHSO₂(C₁-C₄ alkyl), —S(C₁-C₆ alkyl) and —SO₂(C₁-C₆ alkyl), wherein each of said C₁-C₄ alkyl and C₁-C₆ alkyl moieties in the foregoing R⁵ groups may optionally be substituted with one to three fluorine atoms;
R₆ is hydrogen, C₁-C₄ alkyl, fluoro, chloro, bromo, iodo, —CH₂OH, —CH₂OCH₃, or C₁-C₄ alkoxy;
R₇ is hydrogen, C₁-C₄ alkyl, fluoro, chloro, bromo, iodo, —O(C₁-C₄ alkyl), cyano, —CH₂OH, —CH₂O(C₁-C₂ alkyl), —CO(C₁-C₂ alkyl), or —COO(C₁-C₂ alkyl);
R₁₁ is hydrogen, hydroxy, fluoro, or methoxy; and
R₁₂ is hydrogen or C₁-C₄ alkyl;
with the proviso that when A is N, then: (a) B is not unsubstituted alkyl; (b) R₅ is not unsubstituted phenyl or monosubstituted phenyl; and (c) R₃ is not unsubstituted alkyl;
or a pharmaceutically acceptable salt of such compound.

6. A pharmaceutical composition according to claim 1 wherein said corticotropin releasing factor antagonist is a compound of formula 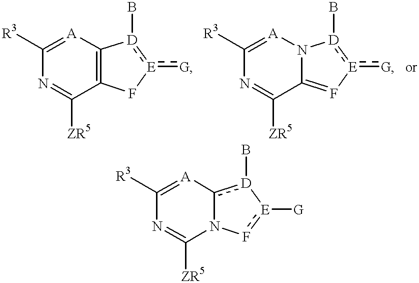
or a pharmaceutically acceptable salt thereof, wherein
the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰, NHR¹, —CR²R¹⁰OR¹, —CR²R¹⁰SR¹ or —COR²;
D is nitrogen and is single bonded to all atoms to which it is attached, or D is carbon and is either double bonded to E in formulas I and II or double bonded to the adjacent carbon atom common to both fused rings in formula III, or D is CH and is single bonded to E in formulas I and II;
E is nitrogen, CH or carbon;
F is oxygen, sulfur, CHR⁴ or NR⁴ when it is single bonded to E and F is nitrogen or CR⁴ when it is double bonded to E;
G, when single bonded to E, is hydrogen, C₁-C₄ alkyl, —S(C₁-C₄ alkyl), —O(C₁-C₄ alkyl), NH₂, —NH(C₁-C₄ alkyl) or —N(C₁-C₂ alkyl)(C₁-C₄ alkyl), wherein each of the C₁-C₄ alkyl groups of G may optionally be substituted with one hydroxy, —O(C₁-C₂ alkyl) or fluoro group; G, when double bonded to E, is oxygen, sulfur or NH; and G, when E is nitrogen and double bonded to D or F, is absent;
R¹ is hydrogen, C₁-C₆ alkyl optionally substituted with one or two substituents R⁸ independently selected from hydroxy, fluoro, chloro, bromo, iodo, C₁-C₄ alkoxy, CF₃, —C(═O)O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C_{1-C}₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)—CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;
R³ is hydrogen, C₁-C₄ alkyl, —O(C₁-C₄ alkyl), chloro, fluoro, bromo, iodo, —CN, —S(C₁-C₄ alkyl) or —SO₂(C₁-C₄ alkyl) wherein each of the (C₁-C₄ alkyl) moieties in the foregoing R³ groups may optionally be substituted with one substituent R⁹ selected from hydroxy, fluoro and (C₁-C₂ alkoxy);
each R⁴ is, independently, hydrogen, (C₁-C₆ alkyl), fluoro, chloro, bromo, iodo, hydroxy, cyano, amino, nitro, —O(C₁-C₄ alkyl), —N(C₁-C₄ alkyl)(C₁-C₂ alkyl), —S(C₁-C₄ alkyl), —SO(C₁-C₄ alkyl), —SO₂(C₁-C₄)alkyl, —CO(C₁-C₄ alkyl), —C(═O)H or —C(═O)O(C₁-C₄alkyl), wherein each of the (C₁-C₆ alkyl) and (C₁-C₄ alkyl) moieties in the foregoing R⁴ groups may optionally contain one or two double or triple bonds and may optionally be substituted with one or two substituents independently selected from hydroxy, amino, C₁-C₃ alkoxy, dimethylamino, methylamino, ethylamino, —NHC(═O)CH₃, fluoro, chloro, C₁-C₃ thioalkyl, —CN, —COOH, —C(═O)O(C₁-C₄ alkyl), —C(═O)(C₁-C₄ alkyl) and —NO₂;
R⁵ is phenyl, naphthyl, thienyl, benzothienyl, pyridyl, quinolyl, pyrazinyl, furanyl, benzofuranyl, benzothiazolyl, benzisothiazolyl, benzisoxazolyl, benzimidazolyl, indolyl, benzoxazolyl or C₃-C₈ cycloalkyl wherein one or two of the carbon atoms of said cycloalkyl rings that contain at least 5 ring members may optionally and independently be replaced by an oxygen or sulfur atom or by NZ⁴ wherein Z⁴ is hydrogen, C₁-C₄ alkyl or benzyl; and wherein each of the foregoing R⁵ groups is substituted with from one to four substituents R¹² wherein one to three of said substituents may be selected, independently, from chloro, C₁-C₆ alkyl and —O(C₁-C₆ alkyl) and one of said substituents may be selected from bromo, iodo, formyl, —CN, —CF₃, —NO₂, —NH₂, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, C₁-C₄alkyl, halo, cyano, hydroxy, —O(C₁-C₄ alkyl) —C(=O)(C₁-C₄ alkyl), —C(═O)O(C₁-C₄ alkyl), —OCF₃, —CF₃, —CH₂0H, —CH₂O (C₁-C₄ alkyl)
R¹⁰ is hydrogen, hydroxy, methoxy or fluoro;
R¹¹ is hydrogen or C₁-C₄ alkyl; and
Z is NH, oxygen, sulfur, —N(C₁-C₄ alkyl), —NC(═O)(C₁-C₂ alkyl), NC(═O)O(C₁-C₂ alkyl) or CR¹³R¹⁴ wherein R¹³ and R¹⁴ are independently selected from hydrogen, trifluoromethyl and methyl with the exception that one of R¹³ and R¹⁴ can be cyano;
with the proviso that: (a) in the five membered rings of structures I, II and III, there can not be two double bonds adjacent to each other; and (b) when R⁴ is attached to nitrogen, it is not halo, cyano or nitro;
or a pharmaceutically acceptable salt of such compound.

7. A pharmaceutical composition according to claim 1 wherein said corticotropin releasing factor antagonist is a compound of formula 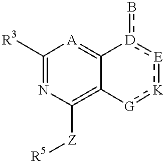
wherein the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR^{R}²R¹⁰ —SCR R²R¹⁰, —CR²R¹⁰NHR¹, —CR²R¹⁰OR¹, —CR²R¹⁰SR¹or —COR², and is single bonded to D; or B is —CR¹R², and is double bonded to D and D is carbon;
D is nitrogen or CR⁴ and is single bonded to all atoms to which it is attached, or D is carbon and is double bonded to E or double bonded to B;
E is oxygen, nitrogen, sulfur, C═O, C═S, CR⁶R¹², NR⁶ or CR⁶; or E is a two atom spacer, wherein one of the atoms is oxygen, nitrogen, sulfur, C═O, C═S, CR⁶R¹², NR⁶ or CR⁶, and the other is CR⁶R¹² or CR⁹;
K and G are each, independently, C═O, C═S, sulfur, oxygen, CHR⁸ or NR⁸ when single bonded to both adjacent ring atoms, or nitrogen or CR⁸ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆alkyl optionally substituted with from one or two substituents independently selected from hydroxy, fluoro, chloro, bromo, iodo, C₁-C₄ alkoxy, CF₃, —C(═O)(C₁-C₄ alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl) (C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)—CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁—C₄ alkyl)(C₁-C₂ alkyl);
—NR¹R² or CR R²R may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ³ wherein Z³ is hydrogen or C₁-C₄ alkyl;
R³ is hydrogen, C₁-C₄ alkyl, —O(C₁—C₄ alkyl), chloro, fluoro, bromo, iodo, —S(C₁-C₄ alkyl) or —SO₂(C₁-C₄ alkyl);
R⁴ is hydrogen, C₁-C₂ alkyl, hydroxy or fluoro;
each R⁶, R⁸ and R⁹ that is attached to a carbon atom is selected, independently, from hydrogen, C₁-C₂ alkyl, fluoro, chloro, bromo, iodo, hydroxy, hydroxymethyl, formyl, trifluoromethyl, cyano, amino, nitro, —O(C₁-C₂ alkyl), —N(C₁-C₂ alkyl)(C₁-C₂ alkyl), —S(C₁-C₂ alkyl), —CO(C₁-C₂ alkyl), —C(═O)H or —C(═O)O (C₁-C₂ alkyl), wherein each of the C₁-C₂ alkyl moieties in the foregoing R⁶, R⁸, and R⁹ groups may optionally contain one double or triple bond; and each R⁶, R⁸, and R⁹ that is attached to a nitrogen atom is selected, independently, from hydrogen and C₁-C₄ alkyl;
R⁵ is substituted phenyl, naphthyl, pyridyl or pyrimidyl, wherein each of the foregoing R⁵ groups is substituted with from two to four substituents R¹⁵, wherein from one to three of said substituents may be selected, independently, from chloro, C₁-C₆ alkyl, —O(C₁-C₆ alkyl) and —(C₁-C₆ alkylene)O(C₁-C₆ alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁C₆ alkyl), and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo, hydroxy, methoxy, —C(═O)(C₁-C₂ alkyl), —C(═O)O(C₁-C₂ alkyl), trifluoromethoxy, hydroxymethyl, trifluoromethyl or formyl;
R¹⁰ is hydrogen, hydroxy, methoxy or fluoro;
R¹¹ is hydrogen or C₁-C₄ alkyl;
R¹² is hydrogen or methyl; and
Z is NH, oxygen, sulfur, —N(C₁-C₄ alkyl), or CR¹³R¹⁴ wherein R¹³ and R¹⁴ are independently selected from hydrogen, and methyl with the exception that one of R¹³ and R¹⁴ may optionally be cyano;
with the proviso that: (a) in the six or seven membered rings of structures in formula 1, there can not be two double bonds adjacent to each other; and (b) when D is carbon and is double bonded to B, then B is CR¹R²;
or a pharmaceutically acceptable salt of such compound.

8. A pharmaceutical composition according to claim 1 wherein said corticotropin releasing factor antagonist is a compound of formula 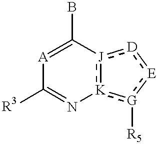
or a pharmaceutically acceptable salt thereof, wherein
the dashed lines represent optional double bonds;
A is nitrogen or CR⁷;
B is —NR¹R²—CR¹R²R¹⁰—C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or—COR²;
J and K are each independently nitrogen or carbon and both J and K are not nitrogens;
D and E are each selected, independently, from nitrogen, CR⁴, C═O, C═S, sulfur, oxygen, CR⁴R⁶ and NR⁸;
G is nitrogen or carbon;
the ring containing D, E, G, K, and J in formula I may be a saturated or unsaturated 5-membered ring and may optionally contain one or two double bonds and may optionally contain from one to three heteroatoms in the ring and may optionally have one or two C═O or C═S groups;
R¹ is C₁-C₆ alkyl optionally substituted with one or two substituents independently selected from hydroxy, fluoro, chloro, bromo, iodo, —O—(C₁-C₄ alkyl), CF₃, —C(═O)O—(C₁-C₄ alkyl), —OC(═O)(C₁-C₄ alkyl), —OC(═O)N (C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), -SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl), alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, —OC(═O)(C₁-C₆ alkyl), —OC(═O)N(C₁-C₄ alkyl) (C₁-C₂ alky), —S(C₁-C₆ alkyl), amino, —NH(C₁-C₂ alkyl), —N(C₁-C₂ alkyl) (C₁-C₄ alkyl), —N(C₁-C₄alkyl)—CO-(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl),
—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;
R³ is hydrogen, C₁-C₄ alkyl, —O(C₁-C₄ alkyl), chloro, fluoro, bromo, iodo, (C₁-C₂ alkylene)—O—(C₁-C₂ alkyl), (C₁-C₂ alkylene)-OH, or —S(C₁-C₄ alkyl);
each R⁴ is, independently, hydrogen, (C₁-C₆ alkyl), fluoro, chloro, bromo, iodo, hydroxy, cyano, amino, (C₁-C₂ alkylene)-OH, CF₃, CH₂SCH₃, nitro, —O(C₁-C₄ alkyl), —N(C₁-C₄ alkyl)(C₁-C₂ alkyl), —S(C₁-C₄ alkyl), —CO(C₁-C₄ alkyl), —C(═O)H or —C(═O)O(C₁-C₄ alkyl);
R⁶ is hydrogen, methyl or ethyl;
R⁸ is hydrogen or C₁-C₄ alkyl;
R⁵ is phenyl, pyridyl, pyrazinyl, pyrimidyl, pyridazinyl and wherein each of the foregoing R⁵ groups is substituted with from one to four substituents R¹³ wherein one to three of said substituents may be selected, independently, from fluoro, chloro, C₁-C₆ alkyl and —O(C₁-C₆ alkyl) and one of said substituents may be selected from bromo, iodo, formyl, OH, (C₁-C₄ alkylene)—OH, (C₁-C₄ alkylene)—O—(C₁-C₂ alkyl), —CN, —CF₃, —NO₂, —NH₂, —NH(C₁-C₄ alkyl), —N(C₁-C₂ alkyl) (C₁-C₆ alkyl), —OCO(C₁-C₄ alkyl), (C₁-C₄ alkylene)—O—(C₁-C₄ alkyl), —S(C₁-C₆ alkyl), (C₁-C₄alkylene)—S—(C₁-C₄ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl, and —SO₂(C₁-C₆ alkyl), and wherein each of the C₁-C₄ alkyl and C₁-C₆alkyl moieties in the foregoing R⁵ groups may optionally have one or two double bonds;
R⁷ is hydrogen, C₁-C₄ alkyl, halo (e.g., chloro, fluoro, iodo or bromo), hydroxy, —O(C₁-C₄ alkyl), —C(═O)(C₁-C₄ alkyl), —C(═O)O(C₁-C₄ alkyl), —OCF₃, —CF₃, —CH₂OH or —CH₂O(C₁-C₂ alkyl);
R¹⁰ is hydrogen, hydroxy, methoxy or fluoro;
R¹¹ is hydrogen or C₁-C₄ alkyl; and
with the proviso that: a) when both J and K are carbons and D is CR⁴ and E is nitrogen, then G can not be nitrogen; (b) when both J and K are carbons and D and G are nitrogens, then E can not be CR⁴ or C═O or C═S; (c) when both J and K are carbons and D and E are carbons, then G can not be nitrogen; (d) when G is carbon, it must be double banded to E; and (e) in the ring containing J, K, D, E and G, there can not be two double bonds adjacent to each other;
and the pharmaceutically acceptable salts of such compounds.

9. A pharmaceutical composition according to claim 1 wherein said corticotropin releasing factor antagonist is a compound of formula 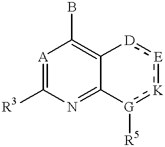
wherein the dashed lines represent optional double bonds;
A is nitrogen or CR⁷;
B is —NR¹R²—CR¹R²R¹⁰, —C(═CR²R¹¹)R¹, —NHCR¹R²R¹⁰, —OCR¹R²R¹⁰—SCR R²R¹⁰, —CR²R¹⁰NHR¹, —CR²R¹⁰OR¹, —CR²R¹⁰SR¹ or —COR²,
G is nitrogen or CR⁴ and is single bonded to all atoms to which it is attached, or G is carbon and is double bonded to K;
K is nitrogen or CR⁶ when double bonded to G or E, or K is oxygen, sulfur, C═O, C═S, CR⁶R¹² or NR⁸ when single bonded to both adjacent ring atoms, or K is a two atom spacer, wherein one of the two ring atoms of the spacer is oxygen, nitrogen, sulfur, C═O, C═S, CR⁶R¹², NR⁶ or CR⁶, and the other is CR⁶R¹² or CR⁹;
D and E are each, independently, C═O, C═S, sulfur, oxygen, CR⁴R⁶ or NR⁸ when single bonded to both adjacent ring atoms, or nitrogen or CR⁴ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄ alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₄ alkyl), each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur atom or by NZ wherein Z is hydrogen, C₁-C₄ alkyl or benzyl, and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)—CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl) (C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ² wherein Z² is hydrogen, benzyl or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
each R⁸, R⁹ and R¹² is selected, independently, from hydrogen and C₁-C₂ alkyl;
each R⁴ and R⁶ that is attached to a carbon atom is selected, independently, from hydrogen and C₁-C₆ alkyl, fluoro, chloro, bromo, iodo, hydroxy, hydroxy (C₁-C₂ alkyl), trifluoromethyl, cyano, amino, nitro, —O(C₁-C₄ alkyl), —N(C₁-C₄ alkyl)(C₁-C₂ alkyl), CH₂SCH₃, —S(C₁-C₄ alkyl), —CO(C₁-C₄ alkyl), -C(═O)H or —C(═O)O(C₁-C₄ alkyl), wherein each of the C₁-C₂ alkyl moieties in the foregoing R⁴ and R⁶ groups may optionally contain one double or triple bond; and R⁶, when attached to a nitrogen atom, is selected from hydrogen and C₁-C₄ alkyl;
R⁵ is substituted phenyl, naphthyl, pyridyl or pyrimidyl, wherein each of the foregoing R⁵ groups is substituted with from two to four substituents R¹³, wherein up to three of said substituents may be selected, independently, from chloro, C₁-C₆ alkyl, —O(C₁-C₆ alkyl) and —(C₁-C₆ alkylene)O(C₁-C₆ alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —(C₀-C₁ alkylene)-S—(C₁-C₂ alkyl), —(C₀-C₁ alkylene)-SO—(C₁-C₂ alkyl), -(C₀-C₁ alkylene)—SO₂—(C₁-C₂ —(C₁-C₄ alkylene)-OH, and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo (e.g., chloro, fluoro, iodo or bromo), hydroxy, methoxy, —C(═O)(C₁-C₂ alkyl), —C(═O)O(C₁-C₂ alkyl), hydroxymethyl, trifluoromethyl or formyl;
R¹⁰ is hydrogen, hydroxy, methoxy or fluoro; and
R¹¹ is hydrogen or C₁-C₄ alkyl;
with the proviso that in the ring containing D, E, K and G of formula I, there can not be two double bonds adjacent to each other;
and the pharmaceutically acceptable salt of such compound.

10. A pharmaceutical composition according to claim 1 wherein said corticotropin releasing factor antagonist is a compound of formula 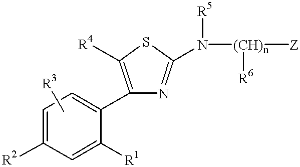
wherein each of R¹ and R² is independently a halogen atom; a C₁-C₅ hydroxyalkyl radical; C₁-C₅ alkyl; C₇-C₁₀ aralkyl; C₁-C₅ alkoxy; trifluoromethyl; nitro; nitrile; a group —SR where R is hydrogen, a C₁-C₅ alkyl radical or a C₇-C₁₀ aralkyl radical; a group S—CO—R where R is a C₁-C₅ alkyl radical or aralkyl in which the aryl portion is C₆-C₈ and the alkyl portion is C₁-C₄; a group —COOR′ where R′ is hydrogen or C₁-C₅ alkyl; a group —CONR′R″ where R′ and R″ are as defined above for R′; a group —NR′R″ where R′ and R″ are as previously defined for R′; a group —CONRaRb or NRaRb, where Ra and Rb, taken together with the nitrogen atom to which they are attached, form a 5- to 7-membered heterocyclic ring; or a group —NHCO—NR′R″, where R′ and R″ are as defined above for R′; R³ is hydrogen or as defined for R¹ and R² is a hydrogen atom; C₁₋₅ alkyl; halogen; a hydroxymethyl group; or a formyl group; R⁵ is C₁-C₅ alkyl; a C₃-C₇ cycloalkyl group; a cycloalkylalkyl group in which the cycloalkyl portion is C₃-C₇ and the alkyl portion is C₁-C₅; or C₅-C₆ alkenyl; n is 0 or 1; R⁶ is C₁₅alkyl; alkoxyalky in which the alkyl portions are C₁-C₅; C₃-C₇ cycloalkyl; a cycloalkylalkyl group in which the cycloalkyl portion is C₃-C₇ and the alkyl portion is C₁-C₅; a cycloalkyloxyalkyl radical in which the cycloalkyl is C₃-C₇ and the alkyl is C₁-C₄; a hydroxyalkyloxyalkyl radical in which the alkyls are C₂-C₁₀; or an alkoxyalkyloxyalkyl radical in which the alkyls are C₃-C₁₂; and Z is an optionally substituted bi- or tricyclic aromatic or heteroaromatic group; and stereoisomers and/or addition salts thereof.

11. A pharmaceutical composition according to claim 1 wherein said corticotropin releasing factor antagonist is a compound of formula 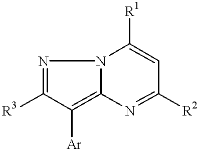
including the stereoisomers and the pharmaceutically acceptable acid addition salt forms thereof, wherein
R¹ is NR⁴R⁵ or OR⁵;
R² is C₁-C₆ alkyl, C₁-C₆ alkyloxy or C₁-C₆ alkylthio,
R³ is hydrogen, C₁-C₆ alkyl, C₁-C₆ alkylsulfonyl, C₁-C₆alkylsulfoxy or C₁-C₆alkylthio;
R⁴ is hydrogen, C₁-C₆ alkyl, mono- or di(C₃-C₆cyloalkylmethyl, C₃-C₆cyloalkyl, C₃-C₆alkenyl, hydroxyC₁-C₆alkyl, C₁-C₆akylcarbonyloxyC₁-C₆alkyl or C₁-C₆ alkyloxyC₁-C₆ alkyl;
R⁵ is C₁-C₈ alkyl, mono- or di(C₃-C₆cycloalkyl)methyl, Ar¹CH₂, C₃-C₆ alkenyl, C₁-C₆ alkyloxyC₁-C₆ alkyl, hydroxyC₁-C₆ alkyl, thienylmethyl, furanylmethyl, C₁-C₆ alkylthioC₁-C₆ alkyl, morpholinyl, mono- or di(C₁-C₆ alkyl)aminoC₁₋₆ alkyl, di(C₁-C₆ alkyl)amino, C₁-C₆ alkylcarbonylC₁-C₆ alkyl, C₁-C₆ alkyl substituted with imidazolyl; or a radical of formula -Alk-O—CO—Ar¹;
or R⁴ and R⁵ taken together with the nitrogen atom to which they are attached may form a pyrrolidinyl, piperidinyl, homopiperidinyl or morpholinyl group, optionally substituted with C₁-C₆ alkyl or C₁-C₆ alkyloxy C₁-C₆ alkyl; and
Ar is phenyl; phenyl substituted with 1, 2 or 3 substituents independently selected from halo, C₁-C₆ alkyl, trifluoromethyl, hydroxy, cyano, C₁-C₆ alkyloxy, benzyloxy, C₁-C₆ alkylthio, nitro, amino and mono- or di(C₁-C₆ alkyl)amino; pyridinyl; pyridinyl substituted with I˜2 or 3 substituents independently selected from halo, C₁-C₆ alkyl, trifluoromethyl, hydroxy, cyano, C₁-C₆ alkyloxy, benzyloxy, C₁-C₆ alkylthio, nitro, amino, mono- or di(C₁-C₆ alkyl)amino and piperidinyl; and wherein said substituted phenyl may optionally be further substituted with one or more halogens;
Ar¹ is phenyl; phenyl substituted with 1, 2 or 3 substituents each independently selected from halo, C₁-C₆ alkyl, C₁-C₆ alkyloxy, di(C₁-C₆alkyl)aminoC₁-C₆ alkyl, trifluoromethyl and C₁-C₆ alkyl substituted with morpholinyl; or pyridinyl; and Alk is C₁-C₆ alkanediyl;
with the proviso that 5-methyl-3-phenyl-7-(phenylmethoxy)-pyrazolo[1,5-a]-pyrimidine and 2,5-dimethyl-7-(methylamino)-3-phenyl-pyrazolo[1,5-a]pyrimidine are not included.

12. A pharmaceutical composition according to claim 1 wherein said corticotropin releasing factor antagonist is a compound of formula 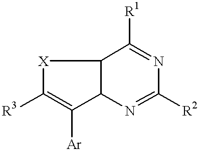
including the stereoisomers and the pharmaceutically acceptable acid addition salt forms thereof, wherein
X is S, SO or S0₂;
R is NR⁴R⁵or OR⁵;
R² is C₁-C₆ alkyl, C₁-C₆ alkyloxy or C₁-C₆ alkylthio;
R³ is hydrogen, C₁-C₆ alkyl, C₁-C₆ alkylsulfonyl, C₁-C₆ alkylsulfoxy or C₁-C₆ alkylthio.
R⁴ is hydrogen, C₁₋₆ alkyl, mono- or di(C₃-C₆cycloalkyl)methyl, C₃-C₆cycloalkyl, C₃-C₆ alkenyl, hydroxyC₁-C₆ alkyl, C₁-C₆ alkylcarbonyloxy C₁-C₆ alkyl or C₁-C₆ alkyloxyC₁-C₆ alkyl;
R⁵ is C₁-C₈ alkyl, mono- or di(C₃-C₆cycloalkyl)methyl, Ar¹CH₂, C₃-C₆ alkenyl, C₁-C₆ alkyloxyC₁-C₆ alkyl, hydroxyC₁-C₆ alkyl, thienyl methyl, furanylmethyl, C₁-C₆ alkythioC₃-C₆ alkyl, morpholinyl, mono- or di(C₁-C₆ alkyl)amino C₁-C₆ alkyl, di(C₁-C₆ alkyl)amino, C₁-C₆ alkylcarbonylC₁-C₆ alkyl, C₁-C₆ alkyl substituted with imidazolyl; or a radical of formula -Alk-O—CO—Ar I;
or R⁴ and R⁵ taken together with the nitrogen atom to which they are attached may form a pyrrolidinyl, piperidinyl, homopiperidinyl or morpholinyl group, optionally substituted with C₁-C₆ alkyl or C₁-C₆ alkyloxyC₁-C₆ alkyl;
Ar is phenyl; phenyl substituted with 1, 2 or 3 substituents independently 5 selected from halo, C₁-C₆ alkyl, trifluoromethyl, hydroxy, cyano, C₁-C₆ alkyloxy, benzyloxy, C₁-C₆ alkylthio, nitro, amino and mono- or di(C₁-C₆ alkyl)amino; pyridinyl;
pyridinyl substituted with 1, 2 or 3 substituents independently selected from halo, C₁-C₆ alkyl, trifluoromethyl, hydroxy, cyano, C₁-C₆ alkyloxy, benzyloxy, C₁-C₆ alkylthio, nitro, amino, mono- or di(C₁-C₆ alkyl)amino and piperidinyl; and wherein said substituted phenyl may optionally be further substituted with one or more halogens;
Ar ¹ is phenyl; phenyl substituted with 1, 2 or 3 substituents each independently selected from halo, C₁-C₆ alkyl, C₁-C₆ alkyloxy, di(C₁-C₆ alkyl)aminoC₁-C₆ alkyl trifluoromethyl, and C₁-C₆ alkyl substituted with morpholinyl; or pyridinyl; and Alk is C₁-C₆ alkanediyl.

13. A pharmaceutical composition according to claim 1 wherein said corticotropin releasing factor antagonist is a compound selected from the group consisting of: 4-(1-ethyl-propoxy)-3,6-dimethyl-2-(2,4,6-trimethylphenoxy)-pyridine;
butyl-[2,5-dimethyl-7-(2,4,6-trimethylphenyl)-6,7-dihydro-5H-pyrrolo[2,3-d]pyrimidin-4-yl]-ethyl-amine;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-NN-(2,4,6-trimethylphenyl)-pyrimidine-4,6-diamine;
[4-(1-ethyl-propoxy)-3,6-dimethyl-pyridin-2-yl]-(2,4,6-trimethylphenyl)-amine;
6-(ethyl-propyl-amino)-2,7-dimethyl-9-(2,4,6-trimethylphenyl)-7,9-dihydro-purin-8-one;
3-{(4-methyl-benzyl)-[3,6-dimethyl-1-(2,4,6-trimethylphenyl)-1H-pyrazolo[3,4-d]pyrimidin-4-yl]-amino}-propan-1-ol;
diethyl-[6-methyl-3-methylsulfanyl-1-(2,4,6-trichlorophenyl)-1H-pyrazolo[3,4-d]pyrimidin-4-yl]-amine;
2-{butyl-[6-methyl-3-methylsulfanyl-1-(2,4,6-trichlorophenyl)-1H-pyrazolo[3,4-d]pyrimidin-4-yl]-amino}-ethanol;
dibutyl-[6-methyl-3-methylsulfanyl-1-(2,4,6-trichlorophenyl)-1H-pyrazolo[3,4-d]pyrimidin-4-yl}-amine;
butyl-ethyl-[6-methyl-3-methylsulfanyl-1-(2,4,6-trichlorophenyl)-1H-pyrazolo[3,4-d]pyrimidin-4-yl]-amine;
butyl-ethyl-[6-methyl-3-methylsulfonyl-1-(2,4,6-trichlorophenyl)-1H-pyrazolo[3,4-d]pyrimidin-4-yl]-amine;
butyl-cyclopropylmethyl-[6-methyl-3-methylsulfanyl-1-(2,4,6-trichlorophenyl)-1H-pyrazolo[3,4-d]pyrimidin-4-yl]-amine;
di-1-propyl-[6-methyl-3-methylsulfanyl-1-(2,4,6-trichlorophenyl)-1H-pyrazolo[3,4-d]pyrimidin-4-yl]-amine;
diallyl-[6-methyl-3-methylsulfanyl-1-(2,4,6-trichlorophenyl)-1H-pyrazolo[3,4-d]pyrimidin-4-yl]-amine;
butyl-ethyl-[6-chloro-3-methylsulfanyl-1-(2,4,6-trichlorophenyl)-1H-pyrazolo[3,4-d]pyrimidin-4-yl]-amine;
butyl-ethyl-[6-methoxy-3-methylsulfanyl-1-(2,4,6-trichlorophenyl)-1H-pyrazolo[3,4-d]pyrimidin-4-yl]-amine;
propyl-ethyl-[3,6-dimethyl-1-(2,4,6-trimethylphenyl)-1H-pyrazolo[3,4-d]pyrimidin-4-yl]-amine;
4-(1-ethyl-propyl)-6-methyl-3-methylsulfanyl-1-(2,4,6-trimethylphenyl)-1H-pyrazolo[3,4-d]pyrimidine;
n-butyl-ethyl-[2,5-dimethyl-7-(2,4,6-trimethylphenyl)-7H-pyrrolo[2,3-d]pyrimidin-4-yl]amine;
di-n-propyl-[2,5-dimethyl-7-(2,4,6-trimethylphenyl)-7H-pyrrolo[2,3-d]pyrimidin-4-yl]amine;
ethyl-n-propyl-[2,5-dimethyl-7-(2,4,6-trimethylphenyl)-7H-pyrrolo[2,3-d]pyrimidin-4-yl]amine;
diethyl-2,5-dimethyl-7-(2,4,6-trimethylphenyl)-7H-pyrrolo[2,3-d]pyrimidin-4-yl]amine;
n-butyl-ethyl-[2,5,6-trimethyl-7-(2,4,6-trimethylphenyl)-7H-pyrrolo[2,3-d]pyrimidin-4-yl]amine;
2-{N-n-butyl-N-[2,5-dimethyl-7-(2,4,6-trimethylphenyl)-7H-pyrrolo[2,3-d]pyrimidin-4-yl]amino}-ethanol;
4-(1-ethyl-propyl)-2,5,6-trimethyl-7-(2,4,6-trimethylphenyl)-7H-pyrrolo[2,3-d]pyrimidine;
n-butyl-ethyl-[2,5-dimethyl-7-(2,4-dimethylphenyl)-7H-pyrrolo[2,3-d]pyrimidin-4-yl]amine;
2,5-dimethyl-7-(2,4,6-trimethylphenyl)-7H-pyrrolo[2,3-d]pyrimidyl-4-yl]-(1-ethyl-propyl)amine;
butyl-[3,6-dimethyl-1-(2,4,6-trimethylphenyl)-1H-pyrazolo[3,4-b]pyridin-4-yl]-ethylamine;
[3,6-dimethyl-1-(2,4,6-trimethylphenyl)-1H-pyrazolo[3,4,b]pyridin-4-yl]-(1-methoxymethylpropyl)-amine;
4-(1-methoxymethylpropoxy)-3,6-dimethyl-1-(2,4,6-trimethylphenyl)-1H-pyrazolo[3,4-b]pyridine;
(1-ethylpropyl)-[3,5,6-trimethyl-1-(2,4,6-trimethylphenyl)-1H-pyrazolo[3,4-b]pyridin-4-yl]-amine;
4-(1-ethylpropoxy)-2,5-dimethyl-7-(2,4,6-trimethylphenyl)-7H-pyrrolo[2,3-b]pyridine;
4-(1-ethylpropoxy)-2,5,6-trimethyl-7-(2,4,6-trimethylphenyl)-7H-pyrrolo[2,3-b]pyridine;
4-(1-ethylpropoxy)-2,5-dimethyl-7-(2,6-dimethyl4-bromophenyl)-7H-pyrrolo[2,3-b]pyridine;
2,5,6-trimethyl-7-(1-propylbutyl)-4-(2,4,6-trimethylphenoxy)-7H-pyrrolo[2,3-d]pyrimidine;
1-(1-ethylpropyl)-6-methyl-4-(2,4,6-trimethylphenylamino)-1,3-dihydro-imidazo[4,5-c]pyridin-2-one;
9-(1-ethylpropyl)-2-methyl-6-(2,4,6-trimethylphenylamino)-7,9-dihydro-purin-8-one;
1-(1-ethylpropyl)-6-methyl-4-(2,4,6-trimethylphenoxy)-1,3-dihydro-imidazo[4,5-c]pyridin-2-one;
1-(1-ethylpropyl)-6-methyl-4-(2,4,6-trimethylphenoxy)-1H-imidazo[4,5-c]pyridine;
1-(1-ethylpropyl)-3,6-dimethyl-4-(2,4,6-trimethylphenoxy)-1,3-dihydro-imidazo[4,5-c]pyridin-2-one;
1-(1-ethylpropyl)-3,6-dimethyl-4-(2,4,6-trimethylphenylamino)-1,3-dihydro-imidazo[4,5-c]pyridin-2-one;
1-(1-ethyl-propyl)4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,2,3,4-tetrahydro-pyrido[3,4-b]pyrazine;
1-(1-ethyl-propyl)-7-methyl-5-(2,4,6-trimethyl-phenoxy)-1,2,3,4-tetrahydro-pyrido[3,4-b]pyrazine;
1-(1-ethyl-propyl)-7-methyl-2-oxo-5-(2,4,6-trimethyl-phenoxy)-1,2,3,4-tetra-hydro-[1,6]naphthyridine-3-carboxylic acid methyl ester;
1-(1-ethyl-propyl)-7-methyl-2-oxo-5-(2,4,6-trimethyl-phenoxy)-1,2,3,4-tetra-hydro-[1,6]naphthyridine-3-carboxylic acid isopropyl ester;
1-(1-ethyl-propyl)-7-methyl-5-(2,4,6-trimethyl-phenoxy)-3,4-dihydro-1H-[1,6]naphthyridin-2-one;
1-(1-ethyl-propyl)-7-methyl-5-(2,4,6-trimethyl-phenoxy)-1,2,3,4-tetrahydro-[1,6]naphthyridine;
1-(1-ethyl-propyl)-7-methyl-5-(2,4,6-trimethyl-phenoxy)-1,4-dihydro-2H-3-oxa-1,6-diaza-naphthalene;
1-(1-ethyl-propyl)-4,7-dimethyl-5-(2,4,6-trimethyl-phenoxy)-1,4-dihydro-2H-3-oxa-1,6-diaza-naphthalene;
1-(1-ethyl-propyl)-3,7-dimethyl-5-(2,4,6-trimethyl-phenoxy)-3,4-dihydro-1H-3-oxa-[1,6]-naphthyridin-2-one;
1-(1-ethyl-propyl)-3,3,6-trimethyl-4-(2,4,6-trimethyl-phenoxy)-2,3-dihydro-1H-pyrrolo[3,2-c]pyridine;
7-(1-ethyl-propoxy)-5-methyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-(1-ethyl-propyl)-amine;
(1-ethyl-propyl)-[5-methyl-3-(2,4,6-trimethyl-phenyl)-pyrazolo[1,5-a]pyrimidin-7-yl]-amine;
7-(1-ethyl-propoxy)-2,5-dimethyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-ethyl-propyl-amine;
[6-bromo-5-bromomethyl-3-(2,4,6-trimethyl-phenyl)-3H-[1,2,3]triazolo[4,5-b]pyridin-7-yl]-(1-ethyl-propyl)-amine;
(1-ethyl-propyl)-[5-methyl-3-(2,4,6-trimethyl-phenyl)-3H-[1,2,3]triazolo[4,5-b]pyridin-7-yl]-amine;
[6-bromo-5-methyl-3-(2,4,6-trimethyl-phenyl)-3H-[1,2,3]triazolo[4,5-b]pyridin-7-yl]-(1-ethyl-propyl)-methyl-amine;
7-(1-ethyl-propoxy)-5-methyl-3-(2,4,6-trimethyl-phenyl)-3H-[1,2,3]triazolo[4,5-b]pyridine;
4-(1-ethyl-propoxy)-2,5-dimethyl-7-(2,4,6-trimethyl-phenyl)-5H-pyrrolo[3,2-d]pyrimidine;
(+)-2,5-dimethyl-4-(tetrahydro-furan-3-yloxy)-7-(2,4,6-trimethyl-phenyl)-5H-pyrrolo-[3,2-d]pyrimidine;
2,5-dimethyl-4-(S)-(tetrahydro-furan-3-yloxy)-7-(2,4,6-trimethyl-phenyl)-5H-pyrrolo-[3,2-d]pyrimidine;
2,5-dimethyl-4-(1-propyl-butoxy)-7-(2,4,6-trimethyl-phenyl)-5H-pyrrolo[3,2-d]pyrimidine;
4-sec-butylsulfanyl-2,5-dimethyl-7-(2,4,6-trimethyl-phenyl)-5H-pyrrolo[3,2-d]pyrimidine;
4-(butyl-ethyl-amino)-2,6-dimethyl-8-(2,4,6-trimethyl-phenyl)-5,8-dihydro-6H-pyrido[2,3-d]pyrimidin-7-one;
8-(1-ethyl-propoxy)-6-methyl-4-(2,4,6-trimethyl-phenyl)-3,4-dihydro-1H-pyrido[2,3-b] pyrazin-2-one;
8-(1-ethyl-propoxy)-6-methyl-4-(2,4,6-trimethyl-phenyl)-1,2,3,4-tetrahydro-pyrido [2,3-b]pyrazine;
4-(1-ethyl-propoxy)-2-methyl-8-(2,4,6-trimethyl-phenyl)-quinoline;
5-(1-ethyl-propoxy)-7-methyl-1-(2,4,6-trimethyl-phenyl)-1,4-dihydro-2H-3-oxa-1,8-diaza-naphthalene;
5-(1-ethyl-propoxy)-7-methyl-1-(2,4,6-trimethyl-phenyl)-1,2-dihydro-3-oxa-1,8-diaza-naphthalen-4-one;
8-(1-ethyl-propoxy)-1,6-dimethyl-4-(2,4,6-trimethyl-phenyl)-1,2,3,4-tetrahydro-pyrido[2,3-b] pyrazine;
(1-ethyl-propyl)-[2-methyl-8-(2,4,6-trimethyl-phenyl)-quinoin-4-yl]-amine;
4-(butyl-ethyl-amino)-2,6-dimethyl-8-(2,6-dimethyl-4-bromo-phenyl)-5,8-dihydro-6H-pyrido[2,3-d]pyrimidin-7-one;
4-(butyl-ethyl-amino)-2-methyl-8-(2,6-dimethyl-4-bromo-phenyl)-5,8-dihydro-6H-pyrido[2,3-d]pyrimidin-7-one;
4-(1-ethyl-propoxy)-2-methyl-8-(2,6-dimethyl-4-bromo-phenyl)-5,8-dihydro-6H-pyrido[2,3-d]pyrimidin-7-one;
(butyl-ethyl)-[2-methyl-8-(2,6-dimethyl-4-bromo-phenyl)-5 ,6 ,7,8-tetrahydro-pyrido[2,3-d]pyrimidin-4-yl]-amine;
(propyl-ethyl)-[2-methyl-8-(2,6-dimethyl-4-bromo-phenyl)-5,6,7,8-tetrahydro-pyrido[2,3-d]pyrimidin-4-yl]-amine;
(diethyl)-[2-methyl-8-(2,6-dimethyl4-bromo-phenyl)-5,6,7,8-tetrahydro-pyrido[2,3-d]pyrimidin-4-yl]-amine;
(1-ethyl-propyl)-[2-methyl-8-(2,6-dimethyl-4-bromo-phenyl)-5,6,7,8-tetrahydro-pyrido[2,3-d]pyrimidin-4-yl]-amine;
(1-ethyl-propoxy)-2-methyl-8-(2,6-dimethyl4-bromo-phenyl)-5,6,7,8-tetrahydro-pyrido[2,3-d]pyrimidine;
4-(butyl-ethyl-amino)-2-methyl-8-(2,4,6-trimethyl-phenyl)-5,8-dihydro-6H-pyrido[2,3-d]pyrimidin-7-one;
4-(1-ethyl-propoxy)-2-methyl-8-(2,4,6-trimethyl-phenyl)-5,8-dihydro-6H-pyrido[2,3-d]pyrimidin-7-one;
(butyl-ethyl)-[2-methyl-8-(2,4,6-trimethyl-phenyl)-5,6,7,8-tetrahydro-pyrido[2,3-d] pyrimidin-4-yl]-amine;
(propyl-ethyl)-[2-methyl-8-(2,4,6-trimethyl-phenyl)-5,6,7,8-tetrahydro-pyrido-[2,3-d] pyrimidin-4-yl]-amine;
(diethyl)-[2-methyl-8-(2,4,6-trimethyl-phenyl)-5,6,7,8-tetrahydro-pyrido[2,3-d] pyrimidin4-yl]-amine;
(1-ethyl-propyl)-[2-methyl-8-(2,4,6-trimethyl-phenyl)-5,6,7,8-tetrahydro-pyrido[2,3-d] pyrimidin4-yl]-amine;
(1-ethyl-propoxy)-2-methyl-8-(2,4,6-trimethyl-phenyl)-5,6,7,8-tetrahydro-pyrido[2,3-d] pyrimidine;
8-(1-ethyl-propoxy)-6-methyl-4-(2,6-dimethyl-4-bromo-phenyl)-3,4-dihydro-1H-pyrido [2,3-b]pyrazin-2-one;
8-(1-ethyl-propoxy)-6-methyl-4-(2,6-dimethyl-4-bromo-phenyl)-1,2,3,4-tetrahydro-pyrido[2,3-b]pyrazine;
4-(1-ethyl-propoxy)-2-methyl-8-(2,6-dimethyl-4-bromo-phenyl)-quinoline;
5-(1-ethyl-propoxy)-7-methyl-1-(2,6-dimethyl4-bromo-phenyl)-1,4-dihydro-2H-3-oxa-1,8-diaza-naphthalene;
5-(1-ethyl-propoxy)-7-methyl-1-(2,6-dimethyl-4-bromo-phenyl)-1,2-dihydro-3-oxa-1,8-diaza-naphthalen-4-one;
8-(1-ethyl-propoxy)-1,6-dimethyl-4-(2,6-dimethyl-4-bromo-phenyl)-1,2,3,4-tetrahydro-pyrido[2,3-b]pyrazine;
(1-ethyl-propyl)-[2-methyl-8-(2,6-dimethyl4-bromo-phenyl)-quinolin-4-yl]-amine;
4-(butyl-ethyl-amino)-2,6-dimethyl-8-(2,6-dimethyl-4-chloro-phenyl)-5,8-dihydro-6H-pyrido[2,3-d]pyrimidin-7-one;
8-(1-ethyl-propoxy)-6-methyl-4-(2,6-dimethyl-4-chloro-phenyl)-3,4-dihydro-1H-pyrido[2,3-b]pyrazin-2-one;
8-(1-ethyl-propoxy)-6-methyl4-(2,6-dimethyl-4-chloro-phenyl)-1,2,3,4-tetrahydro- pyrido[2,3-b]pyrazine;
4-(1-ethyl-propoxy)-2-methyl-8-(2,6-dimethyl-4-chloro-phenyl)-quinoline;
5-(1-ethyl-propoxy)-7-methyl-1-(2,6-dimethyl-4-chloro-phenyl)-1,4-dihydro-2H-3-oxa-1,8-diaza-naphthalene;
5-(1-ethyl-propoxy)-7-methyl-1-(2,6-dimethyl-4-chloro-phenyl)-1,2-dihydro-3-oxa-1,8-diaza-naphthalen-4-one;
8-(1-ethyl-propoxy)-1,6-dimethyl4-(2,6-dimethyl-4-chloro-phenyl)-1,2,3,4-tetrahydro-pyrido[2,3-b]pyrazine;
(1-ethyl-propyl)-[2-methyl-8-(2,6-dimethyl4-chloro-phenyl)-quinolin-4-y]-amine;
8-(1-hydroxymethyl-propoxy)-6-methyl-4-(2,4,6-trimethyl-phenyl)-3,4-dihydro-1H-pyrido[2,3-b]pyrazin-2-one;
8-(1-hydroxymethyl-propylamino)-6-methyl-4-(2,4,6-trimethyl-phenyl)-3,4-dihydro-1H-pyrido[2,3-b]pyrazin-2-one;
8-(1-ethyl-propylamino)-6-methyl4-(2,4,6-trimethyl-phenyl)-3,4-dihydro-1H-pyrido[2,3-b]pyrazin-2-one;
8-diethylamino-6-methyl-4-(2,4,6-trimethyl-phenyl)-3,4-dihydro-1H-pyrido-[2,3-b] pyrazin-2-one;
8-(ethyl-propyl-amino)-6-methyl4-(2,4,6-trimethyl-phenyl)-3,4-dihydro-1H-pyrido[2,3-b]pyrazin-2-one;
8-(butyl-ethyl-amino)-6-methyl-4-(2,4,6-trimethyl-phenyl)-3,4-dihydro-1H-pyrido [2,3-b]pyrazin-2-one;
8-(1-hydroxymethyl-propoxy)-6-methyl4-(2,4,6-trimethyl-phenyl)-1,2,3,4-tetrahydro-pyrido[2,3-b]pyrazine;
8-(1-hydroxymethyl-propylamino)-6-methyl4-(2,4,6-trimethyl-phenyl)-1,2,3,4-tetrahydro-pyrido[2,3-b]pyrazine;
8-(1-ethyl-propylamino)-6-methyl-4-(2,4,6-trimethyl-phenyl)-1,2,3,4-tetrahydro-pyrido[2,3-b]pyrazine;
8-diethylamino-6-methyl-4-(2,4,6-trimethyl-phenyl)-1,2,3,4-tetrahydro-pyrido[2,3-b]pyrazine;
8-(ethyl-propyi-amino)-6-methyl-4-(2,4,6-trimethyl-phenyl)-1,2,3,4-tetrahydro-pyrido[2,3-b]pyrazine;
8-(butyl-ethyl-amino)-6-methyl-4-(2,4,6-trimethyl-phenyl)-1,2,3,4-tetrahydro-pyrido[2,3-b]pyrazine;
4-(1-hydroxymethyl-propoxy)-2-methyl-8-(2,4,6-trimethyl-phenyl)-quinoline;
4-(1-hydroxymethyl-propylamino)-2-methyl-8-(2,4,6-trimethyl-phenyl)-quinoline;
4-(1-ethyl-propylamino)-2-methyl-8-(2,4,6-trimethyl-phenyl)-quinoline;
4-diethylamino-2-methyl-8-(2,4,6-trimethyl-phenyl)-quinoline;
4-(ethyl-propyl-amino)-2-methyl-8-(2,4,6-trimethyl-phenyl)-quinoline;
4-(butyl-ethyl-amino)-2-methyl-8-(2,4,6-trimethyl-phenyl)-quinoline;
5-(1-hydroxymethyl-propoxy)-7-methyl-1-(2,4,6-trimethyl-phenyl)-1,4-dihydro-2H-3-oxa-1,8-diaza-naphthalene;
5-(1-hydroxymethyl-propylamino)-7-methyl-1-(2,4,6-trimethyl-phenyl)-1,4-dihydro-2H-3-oxa-1,8-diaza-naphthalene;
5-(1-ethyl-propylamino)-7-methyl-1-(2,4,6-trimethyl-phenyl)-1,4-dihydro-2H-3-oxa-1,8-diaza-naphthalene;
5-diethylamino-5-methyl-1-(2,4,6-trimethyl-phenyl)-1,4-dihydro-2H-3-oxa-1,8-diaza-naphthalene;
5-(ethyl-propyl-amino)-7-methyl-1-(2,4,6-trimethyl-phenyl)-1,4-dihydro-2H-3-oxa-1,8-diaza-naphthalene;
8-(butyl-ethyl-amino)-6-methyl-4-(2,4,6-trimethyl-phenyl)-1,4-dihydro-2H-3-oxa-1,8-diaza-naphthalene;
4-(2,4-dichlorophenyl)-5-methyl-2-[N-(1-(methoxymethyl)-1-(naphth-2-yl) methyl)-N-propylamino]thiazole;
oxalate of 4-(2,4-dichlorophenyl)-5-methyl-2-[N-(6-methoxyisoquinol-5-yl)-N-propylamino]thiazole;
oxalate of 4-(2-chloro4-methoxyphenyl)-5-methyl-2-[N-(6-methylisoquinol-5-yl)-N-propylamino]thiazole;
4-(2-chloro-4-methoxyphenyl)-5-methyl-2-[N-(1-methoxycarbonylmethylindol-5-yl)-N-propylamino]thiazole;
oxalate of 4-(2-chloro-4-methoxyphenyl)-5-methyl-2-[N-(6-methoxyisoquinol-5-yl)-N-propylamino]thiazole;
oxalate of 4-(2-chloro-4-methoxyphenyl)-5-methyl-2-[N-(6-chloroisoquinol-5-yl)-N-propylamino]thiazole;
oxalate of 4-(2-chloro-4-methoxyphenyl)-5-methyl-2-[N-(6-methoxyisoquinol-5-yl)-N-propylamino]thiazole;
4-(2-chloro-4-methoxyphenyl)-5-methyl-2-[N-1-methoxynaphth-2-yl)-N-propylamino]thiazole;
oxalate of 4-(2-chloro4-trifluoromethylphenyl)-5-methyl-2-[N-6-methoxyisoquinol-5-yl)-N-propylamino]thiazole;
chlorhydrate of 4-(2-chloro4-methoxyphenyl)-5-methyl-2-[N-(2-ethoxynaphth-1-yl)-N- propylamino]thiazole;
chlorhydrate of 4-(2-chloro4-methoxyphenyl)-5-methyl-2[N-(2,3-dimethylnaphth-1-yl)-N-propylamino]thiazole;
chlorhydrate of 4-(2-chloro-4-methoxyphenyl)-5-methyl-2-[N-(6-bromo-2-methoxynaphth-1-yl)-N-propylamino]thiazole;
chlorhydrate of 4-(2-chloro-4-methoxyphenyl)-5-methyl-2-[N-(2,6-dimethylnaphth-1-yl)-N-propylamino]thiazole;
chlorhydrate of 4-(2-chloro-4-methoxyphenyl)-5-methyl-2-[N-(1-(methoxymethyl)-1-(naphth-2-yl)methyl)-N-propylamino]thiazole;
chlorhydrate of 4-(2-chloro-4-methoxyphenyl)-5-methyl-2-[N-(1-(cyclopropyl)-1-(naphth-2-yl)methyl)-N-propylamino]thiazole;
3-(2,4-dichlorophenyl)-5-methyl-7(N-propyl-N-cyclopropanemethylamino)-pyrazolo[2,3-a]pyrimidine;
3-(2,4-dichlorophenyl)-5-methyl-7-(N-allyl-N-cyclopropanemethylamino)-pyrazolo[2,3-a]pyrimidine;
2-methylthio-3-(2,4-dichlorophenyl)-5-methyl-7-(N,N-diallylamino)-pyrazolo[2,3-a]pyrimidine;
2-methylthio-3-(2,4-dichlorophenyl)-5-methyl-7-(N-butyl-N-cyclopropane-methyl-amino)pyrazolo[2,3-a]pyrimidine;
2-methylthio-3-(2,4-dichlorophenyl)-5-methyl-7-(N-propyl-N-cyclopropane-methyl-amino)pyrazolo[2,3-a]pyrimidine;
2-methyl-3-(4-chlorophenyl)-5-methyl-7-(N, N-dipropylamino)-pyrazolo[2,3-a]pyrimidine;
3-[6-(dimethylamino)-3-pyridinyl-2,5-dimethyl-N, N-dipropylpyrazolo[2,3-a]pyrimidin-7-amine;
3-[6-(dimethylamino)-4-methyl-3-pyridinyl]-2,5-dimethyl-N,N-dipropyl-pyrazolo[2,3-a]pyrimidine-7-amine;
3-(2,4-dimethoxyphenyl)-2,5-dimethyl-7-(N-propyl-N-methyloxyethylamino)-pyrazolo(2,3-a)pyrimidine;
7-(N-diethylamino)-2,5-dimethyl-3-(2-methyl-4-methoxyphenyl-[1,5-a]-pyrazolopyrimidine;
7-(N-(3-cyanopropyl)-N-propylamino-2,5,dimethyl-3-(2,4-dimethylphenyl)-[1,5-a]-pyrazolopyrimidine;
[3,6-dimethyl-2-(2,4,6-trimethyl-phenoxy)-pyridin-4-yl]-(1-ethyl-propyl)-amine;
[2-(4-chloro-2,6-dimethyl-phenoxy)-3,6-dimethyl-pyridin-4-yl]-(1-ethyl-propyl)-amine;
cyclopropylmethyl-[3-(2,4-dimethyl-phenyl)-2,5-dimethyl-pyrazolo[1,5-a]pyrimidin-7-yl]-propyl-amine;
cyclopropylmethyl-[3-(2-methyl-4-chloro-phenyl)-2,5-dimethyl-pyrazolo[1,5-a]pyrimidin-7-yl]-propyl-amine;
cyclopropylmethyl-[3-(2,4-di-chloro-phenyl)-2,5-dimethyl-pyrazolo[1,5-a]pyrimidin-7-yl]-propyl-amine;
[3-(2-methyl-4-chloro-phenyl)-2,5-dimethyl-pyrazolo[1,5-a]pyrimidin-7-yl]-di-propyl-amine;
[2,5-dimethyl-3-(2,4-dimethyl-phenyl)-pyrazolo[1,5-a]pyrimidin-7-yl]-(1-ethyl-propyl)-amine;
[2,5-dimethyl-3-(2,4-dichloro-phenyl)-pyrazolo[1,5-a]pyrimidin-7-yl]-(1-ethyl-propyl)-amine;
4-(1-ethyl-propylamino)-6-methyl-2-(2,4,6-trimethyl-phenoxy)-nicotinic acid methyl ester;
3-[6-(dimethylamino)-4-methyl-3-pyridinyl]-2,5-dimethyl-N-propyl-N-cyclopropylmethyl-pyrazolo[2,3-a]pyrimidin-7-amine; and
3-[6-(dimethylamino)-4-methyl-3-pyridinyl]-2,5-dimethyl-N-ethyl-N- cyclopropylmethyl-pyrazolo[2,3-a]pyrimidin-7-amine.

14. A pharmaceutical composition according to claim 13 wherein said corticotropin releasing factor antagonist is a compound selected from the group consisting of:
4-(1-ethyl-propoxy)-3,6-dimethyl-2-(2,4,6-trimethylphenoxy)-pyridine;
4-(1-ethylpropoxy)-2,5-dimethyl-6-(2,4,6-trimethylphenoxy)-pyrimidine;
[4-(1-ethyl-propoxy)-3,6-dimethyl-pyridin-2-yl]-(2,4,6-trimethylphenyl)-amine;
3-{(4-methyl-benzyl)-[3,6-dimethyl-1-(2,4,6-trimethylphenyl)-1H-pyrazolo[3,4-d]pyrimidin-4-yl]-amino}-propan-1-ol;
propyl-ethyl-[3,6-dimethyl-1-(2,4,6-trimethylphenyl)-1H-pyrazolo[3,4-d]pyrimidin-4-yl]-amine;
ethyl-n-propyl-[2,5-dimethyl-7-(2,4,6-trimethylphenyl)-7H-pyrrolo[2,3-d]pyrimidin-4-yl]amine;
2-{N-n-butyl-N-[2,5-dimethyl-7-(2,4,6-trimethylphenyl)-7H-pyrrolo[2,3-d]pyrimidin-4-yl]amino}-ethanol;
[3,6-dimethyl-1-(2,4,6-trimethylphenyl)-1H-pyrazolo[3,4, b]pyridin-4-yl]-(1-methoxymethylpropyl)-amine;
4-(1-ethylpropoxy)-2,5-dimethyl-7-(2,4,6-trimethylphenyl)-7H-pyrrolo[2,3-b]pyridine;
2,5,6-trimethyl-7-(1-propylbutyl)-4-(2,4,6-trimethylphenoxy)-7H-pyrrolo[2,3-d]pyrimidine;
1-(1-ethylpropyl)-6-methyl-4-(2,4,6-trimethylphenoxy)-1,3-dihydro-imidazo[4,5-c]pyridin-2-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,2,3,4-tetrahydro-pyrido[3,4-b]pyrazine;
1-(1-ethyl-propyl)-7-methyl-2-oxo-5-(2,4,6-trimethyl-phenoxy)-1,2,3,4-tetra-hydro-[1,6]naphthyridine-3-carboxylic acid isopropyl ester;
1-(1-ethyl-propyl)-7-methyl-5-(2,4,6-trimethyl-phenoxy)-1,4-dihydro-2H-3-oxa-1,6-diaza-naphthalene;
(1-ethyl-propyl)-[5-methyl-3-(2,4,6-trimethyl-phenyl)-pyrazolo[1,5-a]pyrimidin-7-yl]-amine;
7-(1-ethyl-propoxy)-2,5-dimethyl-3-(2,4,6-trimethyl-phenyl)-pyrazolo[1,5-a]pyrimidine;
4-(1-ethyl-propoxy)-2,5-dimethyl-7-(2,4,6-trimethyl-phenyl)-5H-pyrrolo[3,2-d]pyrimidine;
4-(butyl-ethyl-amino)-2,6-dimethyl-8-(2,4,6-trimethyl-phenyl)-5,8-dihydro-6H-pyrido[2,3-d]pyrimidin-7-one;
8-(1-ethyl-propoxy)-6-methyl-4-(2,4,6-trimethyl-phenyl)-1,2,3,4-tetrahydro-pyrido [2,3-b]pyrazine;
4-(1-ethyl-propoxy)-2-methyl-8-(2,4,6-trimethyl-phenyl)-quinoline;
(1-ethyl-propyl)-[2-methyl-8-(2,4,6-trimethyl-phenyl)-quinolin-4-yl]-amine; -15 (propyl-ethyl)-[2-methyl-8-(2,4,6-trimethyl-phenyl)-5,6,7,8-tetrahydro-pyrido-[2,3-d] pyrimidin-4-yl]-amine;
(1-ethyl-propoxy)-2-methyl-8-(2,4,6-trimethyl-phenyl)-5,6,7,8-tetrahydro-pyrido[2,3-d] pyrimidine;
8-(1-hydroxymethyl-propylamino)-6-methyl-4-(2,4,6-trimethyl-phenyl)-3,4-dihydro-1H-pyrido[2,3-b]pyrazin-2-one;
4-(1-hydroxymethyl-propylamino)-2-methyl-8-(2,4,6-trimethyl-phenyl)-quinoline;
5-(1-hydroxymethyl-propylamino)-7-methyl-1-(2,4,6-trimethyl-phenyl)-1,4-dihydro-2H-3-oxa-1,8-diaza-naphthalene;
[3,6-dimethyl-2-(2,4,6-trimethyl-phenoxy)-pyridin-4-yl]-(1-ethyl-propyl)-amine;
cyclopropylmethyl-[3-(2,4-dimethyl-phenyl)-2,5-dimethyl-pyrazolo[1,5-a]pyrimidin-7-yl]-propyl-amine;
[2,5-dimethyl-3-(2,4-dimethyl-phenyl)-pyrazolo[1,5-a]pyrimidin-7-yl]-(1-ethyl-propyl)-amine;
3-[6-(dimethylamino)-3-pyridinyl-2,5-dimethyl-N,N-dipropylpyrazolo[2,3-a] pyrimidin-7-amine;
3-[6-(dimethylamino)-4-methyl-3-pyridinyl]-2,5-dimethyl-N,N-dipropyl-pyrazolo[2,3-a]pyrimidine-7-amine;
3-(2,4-dimethoxyphenyl)-2,5-dimethyl-7-(N-propyl-N-methyloxyethylamino)-pyrazolo(2,3-a)pyrimidine;
7-(N-diethylamino)-2,5-dimethyl-3-(2-methyl-4-methoxyphenyl-[1,5-a]-pyrazolopyrimidine; and
7-(N-(3-cyanopropyl)-N-propylamino-2,5,dimethyl-3-(2,4-dimethylphenyl)-[1,5-a]-pyrazolopyrimidine.

15. A pharmaceutical composition according to claim 1 wherein said growth hormone secretagogue is a compound of formula IV: 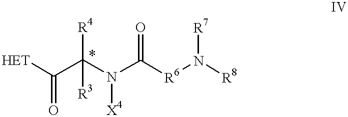
or a stereoisomeric mixture thereof, a diastereomerically enriched, diastereomerically pure, enantiomerically enriched, or enantiomerically pure isomer thereof, or a prodrug of such compound, mixture, or isomer thereof, or a pharmaceutically acceptable salt of the compound, mixture, isomer, or prodrug, wherein:
HET is a heterocyclic moiety selected from the group consisting of [chemical structure]
dis 0, 1,or2;
e is 1 or 2;
f is 0 or 1;
n and w are 0, 1, or 2, provided that n and w cannot both be 0 at the same time;
Y² is oxygen or sulfur;
A is a divalent radical, wherein the left hand side of the radical as shown below is connected to C″ and the right hand side of the radical as shown below is connected to C′, selected from the group consisting of —NR²—CO—NR²—, —NR²—SO₂—NR²—, —O—CO—NR²—, —NR²—CO₂—, —CO—NR²—CO—,—CO—NR²—C(R⁹R¹⁰)—, —C(R⁹R¹⁰)—NR²—CO—,—C(R⁹R¹⁰)—C(R⁹R¹⁰)—C(R⁹R¹⁰)—, —SO₂—C(R⁹R¹⁰)—C(R⁹R¹⁰)—, —C(R⁹R¹⁰)—O—CO—,—C(R⁹R¹⁰)—O—C(R⁹R¹⁰)—, —NR²—CO—C(R⁹R¹⁰)—, —O—CO—C(R⁹R¹⁰)—,l —C(R⁹R¹⁰)—CO—NR²—, —CO—NR²—CO—,—C(R⁹R¹⁰)—CO₂—, —CO—NR²—C(R⁹R¹⁰)—C(R⁹R¹⁰)—, —CO₂—C(R⁹R¹⁰)—, —C(R⁹R¹⁰)—C(R⁹R¹⁰)—C(R⁹R¹⁰)—C(R⁹R¹⁰)—, ——SO₂—NR²—C(R⁹R¹⁰)—, C(R⁹R¹⁰)—,—C(R⁹R¹⁰)—C(R⁹R¹⁰)—NR 2—Co—,—C(R⁹R¹⁰)—C(R⁹R¹⁰)—O—CO—, —NR²—CO—C(R⁹R¹⁰)—C(R⁹R¹⁰)—, —NR²—SO₂—C(R⁹R¹⁰)—C(R⁹R¹⁰)—, —O—CO—C(R⁹R¹⁰)—C(R⁹R¹⁰)—, —C(R⁹R¹⁰)—C(R⁹R¹⁰)—CO—NR², —C(R⁹R¹⁰)—C(R⁹R¹⁰)—CO—, —C(R⁹R¹⁰)—NR²—CO₂—, —C(R⁹R¹⁰)—O—CO—NR², —C(R⁹R¹⁰)—NR²—CO—NR²—, —NR²—CO₂—C(R⁹R¹⁰)—, —NR²—CO—NR²——C(R⁹R¹⁰)—, —NR²—SO₂—NR²—C(R⁹R¹⁰)—, —O—CO—NR²—C(R⁹R¹⁰)—, —CO—N═C(R¹¹)—NR²—, —CO—NR²—C(R¹¹)═N—, —C(R⁹R¹⁰)—NR¹²—C(R⁹R¹⁰)—, —NR¹²—C(R⁹R¹⁰)—, —NR¹²—C(R⁹R¹⁰)—C(R⁹R¹⁰)—, —CO₂—C(R⁹R¹⁰)—C(R⁹R¹⁰)—, —NR²—C(R¹¹)═N—CO—, —C(R⁹R¹⁰)—C(R⁹R¹⁰)—N(R¹²)—, —C(R⁹R¹⁰)—NR¹²—, —N═C(R¹¹)—NR²CO—, —C(R⁹R¹⁰) —C(R⁹R¹⁰)—NR²—SO₂, —C(R⁹R¹⁰)—C(R⁹R¹⁰)—SO₂—NR²—, —C(R⁹R¹⁰)—C(R⁹R¹⁰)—CO₂—, —C(R⁹R¹⁰)—SO₂—C(R⁹R¹⁰)—, —C(R⁹R¹⁰)—C(R⁹R¹⁰)—SO₂—, —O—C(R⁹R¹⁰)—C(R⁹R¹⁰)—, —C(R⁹R¹⁰)—C(R⁹R¹⁰)—O—, —C(R⁹R¹⁰)—CO—C(R⁹R¹⁰)—, —CO—C(R⁹R¹⁰)—C(R⁹R¹⁰)—, and —C(R⁹R¹⁰)—NR²—SO₂—NR²—;
Q is a covalent bond or CH₂;
W is CH or N;
X is CR⁹R¹⁰, C═CH₂, or C═O;
Y is CR⁹R¹⁰, O, or NR²;
Z is C═O, C═S, or SO₂;
G¹ is hydrogen, halo, hydroxy, nitro, amino, cyano, phenyl, carboxyl, —CONH₂, —C₁-C₄ alkyl optionally independently substituted with one or more phenyl, one or more halogen, or one or more hydroxy groups, —C₁-C₄ alkoxy optionally independently substituted with one or more phenyl, one or more halogen, or one or more hydroxy groups, —C₁-C₄ alkylthio, phenoxy, —CO₂—(C₁-C₄ alkyl), N,N-di-(C₁-C₄ alkylamino), —C₂-C₆ alkenyl optionally independently substituted with one or more phenyl, one or more halogen, or one or more hydroxy groups, —C₂-C₆ alkynyl optionally independently substituted with one or more phenyl, one or more halogen, or one or more hydroxy groups, —C₃-C₆ cycloalkyl optionally independently substituted with one or more C₁-C₄ alkyl groups, one or more halogen, or one or more hydroxy groups, —C₁-C₄ alkylamino carbonyl, or di-C₁-C₄ alkylamino) carbonyl;
G² and G³ are each independently selected from the group consisting of hydrogen, halo, hydroxy, —C₁-C₄ alkyl optionally independently substituted with one to three halo groups, and —C₁-C₄ alkoxy optionally independently substituted with one to three halo groups;
R¹ is hydrogen, —CN, —(CH₂)_{q}NX⁶COX⁶, —(CH₂)_{q}NX⁶, —CO(CH₂)ₜ—A¹, —(CH₂)_{q}NX⁶SO₂(CH₂)ₜ—A¹, (CH₂)_{q}NX⁶SO₂X⁶, —(CH₂)_{q}NX⁶CONX⁶(CH₂)ₜ—A¹, —(CH₂)_{q}NX⁶CONX⁶X⁶, —(CH₂)_{q}CONX⁶X⁶, —(CH₂)_{q}CONX⁶(CH₂)ₜ—A¹, —(CH₂)_{q}CO₂X⁶, —(CH₂)_{q}CO₂(CH₂)ₜ—A¹, —(CH₂)_{q}OX⁶, —(CH₂)_{q}OCOX⁶, —(CH₂)_{q}OCO(CH₂)ₜ—A¹, —(CH₂)_{q}OCONX⁶(CH₂)ₜ—A¹, —(CH₂)_{q}OCONX⁶X⁶, —(CH₂)_{q}COX⁶, —(CH₂)_{q}CO(CH₂)ₜ—A¹, —(CH₂)_{q}NX⁶CO₂X⁶, —(CH₂)_{q}NX6SO₂NX⁶X⁶, —(CH₂)_{q}SOₘX⁶, (CH₂)_{q}SOₘ(CH₂)ₜ—A¹, —C₁-C₁₀ alkyl, —(CH₂)ₜ—A¹, —(CH₂)_{q}—(C₃-C₇ cycloalkyl), —(CH₂)_{q}—Y¹—(C₁-C₆ alkyl ), —(CH₂)_{q}—Y¹—(CH₂)ₜ—A¹, or —(CH₂)_{q}—Y¹—(CH₂)ₜ—(C₃-C₇ cycloalkyl);
wherein the alkyl and cycloalkyl groups in the definition of R¹ are optionally substituted with C₁-C₄ alkyl, hydroxy, C₁-C₄ alkoxy, carboxyl, —CONH₂, —SOₘ, —(C₁-C₆ alkyl), —CO₂—(C₁-C₄ alkyl) ester, 1H-tetrazol-5-yl, or 1, 2, or 3 fluoro groups;
Y¹ is O, SOₘ, —CONX⁶—, —CH═CH—, —C≡C—, —NX⁶CO—, —CONX⁶—, —CO₂—, —OCONX⁶— or —OCO—;
q is b 0, 1,2,3, or4;
t is 0, 1, 2, or 3;
said (CH₂)_{q} group and (CH₂)ₜ group in the definition of R¹ are optionally independently substituted with hydroxy, C₁-C₄ alkoxy, carboxyl, —CONH₂, —SOₘ—(C₁-C₆ alkyl), —CO₂—(C₁-C₄ alkyl) ester, 1H-tetrazol-5-yl, 1, 2, or 3 fluoro groups, or 1 or 2 C₁-C₄ alkyl groups;
R^{1A} is selected from the group consisting of hydrogen, F, Cl, Br, I, C₁-C₆ alkyl, phenyl-(C₁-C₃ alkyl), pyridyl-(C₁-C₃alkyl), thiazolyl-(C₁-C₃alkyl), and thienyl-(C₁-C₃ alkyl), provided that R^{1A} is not F, Cl, Br, or I when a heteroatom is vicinal to C″;
R² is hydrogen, C₁-C₈ alkyl, —(C₀-C₃ alkyl)-(C₃-C₈ cycloalkyl), —(C₁-C₄ alkyl)-A¹or A¹, wherein the alkyl groups and the cycloalkyl groups in the definition of R² are optionally substituted with hydroxy, —CO₂X⁶, —CONX⁶X⁶, —NX⁶X⁶, —SOₘ(C₁-C₆ alkyl), —COA¹, —COX⁶, CF₃, CN, or 1, 2, or 3 independently selected halo groups;
R³ is selected from the group consisting of A¹, C₁-C₁₀alkyl, —(C₁-C₆ alkyl)-A¹, —(C₁-C₆ alkyl)-(C₃-C₇ cycloalkyl), —(C₁-C₅ alkyl)-X¹—(C₁-C₅ alkyl), —(C₁-C₅ alkyl)-X¹—(C₀-C₅ alkyl)-A¹, and —(C₁-C₅ alkyl)-X¹—(C₁-C₅ alkyl)-(C₃-C₇ cycloalkyl);
wherein the alkyl groups in the definition of R³ are optionally substituted with —SOₘ(C₁-C₆ alkyl), —CO₂X³, 1, 2, 3, 4, or 5 independently selected halo groups, or 1, 2, or 3 independently selected —OX³ groups;
X¹ is O, SOₘ, —NX²CO—, —CONX²—, —OCO—, —CO₂—, —CX²═CX²—, —NX²CO₂—, —OCONX²—, or —C≡C—;
R⁴ is hydrogen, C₁-C₆ alkyl, or C₃-C₇ cycloalkyl, or R⁴ taken together with R³ and the carbon atom to which they are attached form C₅-C₇ cycloalkyl, C₅-C₇ cycloalkenyl, a partially saturated or fully saturated 4- to 8-membered ring having 1 to 4 heteroatoms independently selected from the group consisting of oxygen, sulfur, and nitrogen, or a bicyclic ring system consisting of a partially saturated or fully saturated 5- or 6-membered ring, fused to a partially saturated, fully unsaturated, or fully saturated 5- or 6-membered ring, optionally having 1 to 4 heteroatoms independently selected from the group consisting of nitrogen, sulfur, and oxygen;
X⁴ is hydrogen or C₁-C₆ alkyl, or X⁴ is taken together with R⁴ and the nitrogen atom to which X⁴ is attached and the carbon atom to which R⁴ is attached and form a five to seven membered ring;
R⁶ is a bond or is 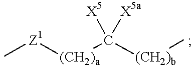
wherein a and b are each independently 0, 1, 2, or 3;
X⁵ and X^{5a} are each independently selected from the group consisting of hydrogen, CF₃, A¹, and C₁-C₆ alkyl optionally substituted with A¹, OX², —SOₘ—(C₁-C₆ alkyl), —CO₂X², C₃-C₇ cycloalkyl, —NX²X², or —CONX²X²;
or the carbon bearing X⁵ or X^{5a} forms one or two alkylene bridges with the nitrogen atom bearing R⁷ and R⁸ wherein each alkylene bridge contains 1 to 5 carbon atoms, provided that when one alkylene bridge is formed then only one of X⁵ or X^{5a} is on the carbon atom and only one of R⁷ or R⁸ is on the nitrogen atom, and further provided that when two alkylene bridges are formed then X⁵ and X^{5a} cannot be on the carbon atom and R⁷ and R⁸ cannot be on the nitrogen atom;
or X⁵ taken together with X^{5a} and the carbon atom to which they are attached form a partially saturated or fully saturated 3- to 7-membered ring, or a partially saturated or fully saturated 4- to 8-membered ring having 1 to 4 heteroatoms independently selected from the group consisting of oxygen, sulfur, and nitrogen;
or X⁵ taken together with X^{5a} and the carbon atom to which they are attached form a bicyclic ring system consisting of a partially saturated or fully saturated 5- or 6-membered ring, optionally having 1 or 2 heteroatoms independently selected from the group consisting of nitrogen, sulfur, and oxygen, fused to a partially saturated, fully saturated, or fully unsaturated 5- or 6-membered ring, optionally having 1 to 4 heteroatoms independently selected from the group consisting of nitrogen, sulfur, and oxygen;
Z¹ is a bond, O, or N—X², provided that when a and b are both 0 then Z¹ is not N—X² or O;
R⁷ and R⁸ are each independently hydrogen or C₁-C₆ alkyl optionally independently substituted with A¹, —CO₂—(C₁-C₆ alkyl), —SOₘ(C₁-C₆ alkyl), 1 to 5 halo groups, 1 to 3 hydroxy groups, 1 to 3 —O—CO(C₁-C₁₀ alkyl) groups, or 1 to 3 C_{-C}₆ alkoxy groups; or
R⁷ and R⁸ can be taken together to form —(CH₂)ᵣ—L—(CH₂)ᵣ—, wherein L is CX²X², SOₘ, or NX²;
R⁹ and R¹⁰ are each independently selected from the group consisting of hydrogen, fluoro, hydroxy, and C₁-C₅ alkyl optionally independently substituted with 1-5 halo groups;
R¹¹ is selected from the group consisting of C₁-C₅ alkyl and phenyl optionally substituted with 1-3 substituents each independently selected from the group consisting of C₁-C₅ alkyl, halo, and C₁-C₅ alkoxy;
R¹² is selected from the group consisting of C₁-C₅ alkylsulfonyl, C₁-C₅ alkanoyl, and C₁-C₅ alkyl wherein the alkyl portion is optionally independently substituted by 1-5 halo groups;
A¹ for each occurrence is independently selected from the group consisting of C₅-C₇ cycloalkenyl, phenyl, a partially saturated, fully saturated, or fully unsaturated 4- to 8-membered ring optionally having 1 to 4 heteroatoms independently selected from the group consisting of oxygen, sulfur, and nitrogen, and a bicyclic ring system consisting of a partially saturated, fully unsaturated, or fully saturated 5- or 6-membered ring, optionally having 1 to 4 heteroatoms independently selected from the group consisting of nitrogen, sulfur, and oxygen, fused to a partially saturated, fully saturated, or fully unsaturated 5- or 6-membered ring, optionally having 1 to 4 heteroatoms independently selected from the group consisting of nitrogen, sulfur, and oxygen;
A¹ for each occurrence is independently optionally substituted, on one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —CONX⁶X⁶, —CO₂X⁶, oxo, C₁-C₆ alkyl, nitro, cyano, benzyl, —SOₘ(C₁-C₆ alkyl), 1H-tetrazol-5-yl, phenyl, phenoxy, phenylalkyloxy, halophenyl, methylenedioxy, —NX⁶X⁶, —NX⁶COX⁶, —SO₂NX⁶X⁶, —NX⁶SO₂-phenyl, NX⁶SO₂X⁶, —CONX¹¹X¹², —SO₂NX¹¹X¹², —NX⁶SO₁₂X¹², —NX⁶CONX¹¹X¹², —NX⁶SO₂NX¹¹X¹², —NX⁶COX¹², imidazolyl, thiazolyl, and tetrazolyl, provided that if A¹ is optionally substituted with methylenedioxy then it can only be substituted with one methylenedioxy;
wherein X¹¹ is hydrogen or C₁-C₆ alkyl optionally independently substituted with phenyl, phenoxy, C₁-C₆ alkoxycarbonyl, —SOₘ(C₁-C₆ alkyl), 1 to 5 halo groups, 1 to 3 hydroxy groups, 1 to 3 C₁-C₁₀ alkanoyloxy groups, or 1 to 3 C₁-C₆ alkoxy groups;
X¹² is hydrogen, C₁-C₆ alkyl, phenyl, thiazolyl, imidazolyl, furyl, or thienyl, provided that when X¹² is not hydrogen, the X¹² group is optionally substituted with one to three substituents independently selected from the group consisting of Cl, F, CH₃, OCH₃, OCF₃, and CF₃;
or X¹¹ and X¹² are taken together to form —(CH₂)ᵣ—L¹—(CH₂)ᵣ—, wherein L¹ is CX²X², O, SOₘ, or NX²;
r for each occurrence is independently 1, 2, or 3;
X² for each occurrence is independently hydrogen, optionally substituted C₁-C₆ alkyl, or optionally substituted C₃-C₇ cycloalkyl, wherein the optionally substituted C₁-C₆ alkyl and optionally substituted C₃-C₇ cycloalkyl in the definition of X² are optionally independently substituted with —SOₘ(C₁-C₆ alkyl), —CO₂X³, 1 to 5 halo groups, or 1-3 OX³ groups;
X³ for each occurrence is independently hydrogen or C₁-C₆ alkyl;
X⁶ for each occurrence is independently hydrogen, optionally substituted C₁-C₆ alkyl, halogenated C₂-C₆ alkyl, optionally substituted C₃-C₇ cycloalkyl, halogenated C₃-C₇ cycloalkyl, wherein the optionally substituted C₁-C₆ alkyl and optionally substituted C₃-C₇ cycloalkyl in the definition of X⁶ are optionally independently mono- or di-substituted with C₁-C₄ alkyl, hydroxy, C₁-C₄ alkoxy, carboxyl, CONH₂, —SOₘ(C₁-C₆ alkyl), carboxylate (C₁-C₄ alkyl) ester, or 1H-tetrazol-5-yl; or when there are two X⁶ groups on one atom and both X⁶ are independently C₁-C₆ alkyl, the two C₁-C₆ alkyl groups may be optionally joined, and together with the atom to which the two X⁶ groups are attached, form a 4- to 9- membered ring optionally having oxygen, sulfur, or NX⁷ as a ring member, wherein X⁷ is hydrogen or C₁-C₆ alkyl optionally substituted with hydroxy;
m for each occurrence is independently 0, 1, or 2; with the provisos that:
X⁶ and X¹² cannot be hydrogen when attached to CO or SO₂ in the form COX⁶, COX¹², SO₂X⁶ or SO₂X¹²; and
when R⁶ is a bond then L is NX² and each r in the definition —(CH₂)ᵣL—(CH₂)ᵣ— is independently 2 or 3.

16. A pharmaceutical composition according to claim 15 wherein said growth hormone secretagogue is
2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide;
2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide;
2-amino-N-{1 (R)-benzyloxymethyl-2-[1,3-dioxo-8a(S)-pyridin-2-ylmethyl-2-(2,2,2-trifluoro-ethyl)-hexahydro-imidazo[1,5-a]pyrazin-7-yl]-2-oxo-ethyl}-2-methyl-propionamide;
N-(1 (R)-((1,2-dihydro-1-methanesulfonyl-spiro(3H-indole-3,4′-piperidin)-1′-yl)carbonyl)-2-(phenylmethyloxy)ethyl)-2-amino-2-methyl-propanamide; or
a prodrug of any of these compounds or a pharmaceutically acceptable salt of any of said compounds or said prodrugs.

17. A pharmaceutical composition according to claim 13 wherein said growth hormone secretagogue is a compound of formula IV: 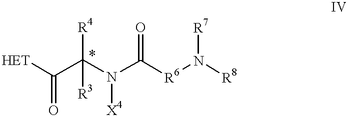
or a stereoisomeric mixture thereof, a diastereomerically enriched, diastereomerically pure, enantiomerically enriched, or enantiomerically pure isomer thereof, or a prodrug of such compound, mixture, or isomer thereof, or a pharmaceutically acceptable salt of the compound, mixture, isomer, or prodrug, wherein:
HET is a heterocyclic moiety selected from the group consisting of [chemical structure]
d is 0,1, or 2;
e is 1 or 2;
f is 0 or 1;
n and w are 0, 1, or 2, provided that n and w cannot both be 0 at the same time;
y² is oxygen or sulfur;
A is a divalent radical, wherein the left hand side of the radical as shown below is connected to C″ and the right hand side of the radical as shown below is connected to C′, selected from the group consisting of —NR²—CO—NR²—, —NR²—SO₂—NR²—, —O—CO—NR²—, —NR²—CO₂—, —CO—NR²—CO—, —CO—NR²—C(R⁹R¹⁰)—, —C(R⁹R¹⁰)—NR²—CO—, —C(R⁹R¹⁰)—C(R⁹R¹⁰)—C(R⁹R¹o)—, —SO₂—C(R⁹R¹⁰)—C(R⁹R¹⁰)—, —C(R⁹R¹⁰)—O—CO—, —C(R⁹R¹⁰)—O—C(R⁹R¹⁰)—, —NR²—CO—C(R⁹R¹⁰)—, —O—CO—C(R⁹R¹⁰)—, —C(R⁹R¹⁰)—CO—NR²—, —CO—NR²—CO—, —C(R⁹R¹⁰)—CO₂—, —CO—NR²—C(R⁹R¹⁰)—C(R⁹R¹⁰)—, —CO₂—C(R⁹R¹⁰)—, —C(R⁹R¹⁰)—C(R⁹R¹⁰)—C(R⁹R¹⁰) —C(R⁹R¹⁰)—, —SO₂ —NR²—C(R⁹R¹⁰)—, C(R⁹R¹⁰)—, —C(R⁹R¹⁰)—C(R⁹R¹⁰)—NR²—CO—, —C(R⁹R¹⁰)—C(R⁹R¹⁰)—O—CO—, NR²—CO—C(R⁹R¹⁰)—C(R⁹R¹⁰)—, —NR²—SO₂—C(R⁹R¹⁰)—C(R⁹¹⁰)—, —O—CO—C(R⁹R¹⁰)—C(R⁹¹⁰)—, —C(R⁹R¹⁰)—C(R⁹R¹⁰)—CO—NR²—, —C(R⁹R¹⁰)—C(R⁹R¹⁰)—CO—, —C(R⁹R¹⁰)—NR²—CO₂—, —C(R⁹R¹⁰)—O—CO—NR², —C(R⁹R¹⁰)—NR²—CO—NR²—, —NR²—CO₂—C(R⁹R¹⁰)—, —NR²—CO—NR²C(R⁹R¹⁰)—, —NR²—SO₂—NR²—C(R⁹R¹⁰)—, —O—CO—NR²—C(R⁹R¹⁰)—, —CO—N═C(R¹¹)—NR²—, —CO—NR²—C(R¹¹)═N—, —C(R⁹R¹⁰)—NR¹²—C(R⁹R¹⁰)—, —NR¹²—C(R⁹R¹⁰)—, —NR¹²—C(R⁹¹⁰)—C(R⁹R¹⁰)—, —CO₂—C(R⁹R¹⁰)—C(R⁹R¹⁰)—, —NR²—C(R¹¹)═N—CO—, —C(R⁹R¹⁰)—C(R⁹R¹⁰)—N(R¹²)—, —C(R⁹R¹⁰)—NR¹²—, —N═C(R¹¹)—NR²—CO—, —C(R⁹R¹⁰)—C(R⁹R¹⁰)—NR²—SO₂—, —C(R⁹R¹⁰)—C(R⁹R¹⁰)—SO₂—NR²—, —C(R⁹R¹⁰)—C(R⁹R¹⁰)—CO₂—, —C(R⁹R¹⁰)—SO₂—C(R⁹R¹⁰)—, —C(R⁹¹⁰)—C(R⁹R¹⁰)—SO₂—, —O—C(R⁹R¹⁰)—C(R⁹R¹⁰)—, —C(R⁹R¹⁰)—C(R⁹R¹⁰)—O—, —C(R⁹R¹⁰)—CO—C(R⁹R¹⁰)—, —CO—C(R⁹R¹⁰)—C(R⁹R¹⁰)—, and —C(R⁹R¹⁰)—NR²—SO₂—NR²—;
Q is a covalent bond or CH₂;
W is CH or N;
X is CR⁹R¹⁰, C═CH₂, or C═O;
Y is CR⁹R¹⁰, O, or NR²;
Z is C═O, C═S, or SO₂;
G¹ is hydrogen, halo, hydroxy, nitro, amino, cyano, phenyl, carboxyl, —CONH₂, —C₁-C₄ alkyl optionally independently substituted with one or more phenyl, one or more halogen, or one or more hydroxy groups, -C₁-C₄ alkoxy optionally independently substituted with one or more phenyl, one or more halogen, or one or more hydroxy groups, —C₁-C₄ alkylthio, phenoxy, CO₂—(C₁-C₄ alkyl), N,N-di-(C₁-C₄ alkylamino), —C₂-C₆ alkenyl optionally independently substituted with one or more phenyl, one or more halogen, or one or more hydroxy groups, —C₂-C₆ alkynyl optionally independently substituted with one or more phenyl, one or more halogen, or one or more hydroxy groups, —C₃-C₆ cycloalkyl optionally independently substituted with one or more C₁-C₄ alkyl groups, one or more halogen, or one or more hydroxy groups, —C₁-C₄ alkylamino carbonyl, or di-C₁—C₄ alkylamino) carbonyl;
G² and G³ are each independently selected from the group consisting of hydrogen, halo, hydroxy, —C₁-C₄ alkyl optionally independently substituted with one to three halo groups, and —C₁-C₄ alkoxy optionally independently substituted with one to three halo groups;
R¹ is hydrogen, —CN, —(CH₂)_{q}NX⁶COX⁶, —(CH₂)_{q}NX⁶CO(CH₂)ₜ—A¹, —(CH₂)_{q}NX⁶SO₂(CH₂)ₜ—A¹, —(CH₂)_{q}NX⁶SO₂X⁶, —(CH₂)_{q}NX⁶CONX⁶(CH₂)ₜ—A¹, —(CH₂)_{q}NX⁶CONX⁶X⁶, —(CH₂)_{q}CONX⁶X⁶, —(CH₂)_{q}CONX⁶(CH₂)ₜ—A¹, —(CH₂)_{q}CO₂X⁶, —(CH₂)_{q}CO₂(CH₂X—A¹, —(CH₂)_{q}OX⁶, —(CH₂)_{q}OCOX⁶, —(CH₂)_{q}OCO(CH₂)ₜ—A¹, —(CH₂)_{q}OCONX⁶(CH₂)ₜ—A¹, —(CH₂)_{q}OCONX⁶X⁶, —(CH₂)_{q}COX⁶, —(CH₂)_{q}CO(CH₂)ₜ—A¹, —(CH₂)_{q}NX⁶CO₂X⁶, —(CH₂)_{q}NX⁶SO₂NX⁶X⁶, —(CH₂)_{q}SOₘX⁶, —(CH₂)_{q}SOₘ(CH₂)ₜ—A¹, —C₁-C₁₀ alkyl, —(CH₂)ₜ—A¹, —(CH₂)_{q}—(C₃- C₇ cycloalkyl), —(CH₂)_{q}—Y¹—(C₁-C₆ alkyl), —(CH₂)_{q}—Y¹—(CH₂)ₜ—A¹, or —(CH₂)_{q}—Y¹—(CH₂)ₜ—(C₃—C₇ cycloalkyl);
wherein the alkyl and cycloalkyl groups in the definition of R¹ are optionally substituted with C₁-C₄ alkyl, hydroxy, C₁-C₄ alkoxy, carboxyl, —CONH₂, —SOₘ—(C₁-C₆ alkyl), —CO₂—(C₁-C₄ alkyl) ester, 1H-tetrazol-5-yl, or 1, 2, or 3 fluoro groups;
Y¹ is O, SOₘ, —CONX⁶—, —CH═CH—, —C≡C—, —NX⁶CO—, —CONX⁶—, —CO₂—, —OCONX⁶— or —OCO—;
q is 0, 1,2,3, or 4;
t is 0, 1, 2, or 3;
said (CH₂)_{q} group and (CH₂)ₜ group in the definition of R¹ are optionally independently substituted with hydroxy, C₁-C₄ alkoxy, carboxyl, -CONH₂, —SOₘ—(C₁-C₆ alkyl), —CO₂—(C₁-C₄ alkyl) ester, 1H-tetrazol-5-yl, 1, 2, or 3 fluoro groups, or 1 or 2 C₁-C₄ alkyl groups;
R^{1A} is selected from the group consisting of hydrogen, F, Cl, Br, I, C₁-C₆ alkyl, phenyl-(C₁-C₃ alkyl), pyridyl-(C₁-C₃alkyl), thiazolyl-(C₁-C₃alkyl), and thienyl-(C₁-C₃ alkyl), provided that R^{1A} is not F, Cl, Br, or I when a heteroatom is vicinal to C″;
R² is hydrogen, C₁-C₈ alkyl, —(C₀-C₃ alkyl)-(C₃-C₈ cycloalkyl), —(C₁-C₄ alkyl)—A¹ or A¹, wherein the alkyl groups and the cycloalkyl groups in the definition of R² are optionally substituted with hydroxy, —CO₂X⁶, —CONX⁶X⁶, —NX⁶X⁶, —SOₘ(C₁-C₆ alkyl), —COA¹, -COX⁶, CF₃, CN, or 1, 2, or 3 independently selected halo groups;
R³ is selected from the group consisting of A¹, C₁-C₁₀alkyl, —(C₁-C₆ alkyl)—A¹, —(C₁-C₆ alkyl)-(C₃-C₇ cycloalkyl), —(C₁-C₅ alkyl)-X¹-(C₁-C₅ alkyl), —(C₁-C₅ alkyl), -X¹—(C₀-C₅ alkyl)-A¹, and -(C₁-C₅ alkyl)-X¹—(C₁-C₅ alkyl)-(C₃-C₇ cycloalkyl);
wherein the alkyl groups in the definition of R³ are optionally substituted with —SOₘ(C₁-C₆ alkyl), —CO₂X³, 1, 2, 3, 4, or 5 independently selected halo groups, or 1, 2, or 3 independently selected -OX³ groups;
X¹ is O, SOₘ, —NX²CO—, —CONX²—, —OCO—, —CO₂—, —CX²═CX²—, —NX²CO₂—, —OCONX²—, or —C≡C—;
R⁴ is hydrogen, C₁-C₆ alkyl, or C₃-C₇ cycloalkyl, or R⁴ taken together with R³ and the carbon atom to which they are attached form C₅-C₇ cycloalkyl, C₅-C₇ cycloalkenyl, a partially saturated or fully saturated 4- to 8-membered ring having 1 to 4 heteroatoms independently selected from the group consisting of oxygen, sulfur, and nitrogen, or a bicyclic ring system consisting of a partially saturated or fully saturated 5- or 6-membered ring, fused to a partially saturated, fully unsaturated, or fully saturated 5- or 6-membered ring, optionally having 1 to 4 heteroatoms independently selected from the group consisting of nitrogen, sulfur, and oxygen;
X⁴ is hydrogen or C₁-C₆ alkyl, or X⁴ is taken together with R⁴ and the nitrogen atom to which X⁴ is attached and the carbon atom to which R⁴ is attached and form a five to seven membered ring;
R⁶ is a bond or is 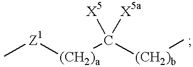
wherein a and b are each independently 0, 1, 2, or 3;
X⁵ and X^{5a} are each independently selected from the group consisting of hydrogen, CF₃, A¹, and C₁-C₆ alkyl optionally substituted with A¹, OX², —SOₘ—(C₁-C₆ alkyl), —CO₂X², C₃-C₇ cycloalkyl, —NX²X², or —CONX²X²;
or the carbon bearing X⁵ or X⁵a forms one or two alkylene bridges with the nitrogen atom bearing R⁷ and R⁸ wherein each alkylene bridge contains 1 to 5 carbon atoms, provided that when one alkylene bridge is formed then only one of X⁵ or X^{5a} is on the carbon atom and only one of R⁷ or R⁸ is on the nitrogen atom, and further provided that when two alkylene bridges are formed then X⁵ and X^{5a} cannot be on the carbon atom and R⁷ and R⁸ cannot be on the nitrogen atom;
or X⁵ taken together with X^{5a} and the carbon atom to which they are attached form a partially saturated or fully saturated 3- to 7-membered ring, or a partially saturated or fully saturated 4- to 8-membered ring having 1 to 4 heteroatoms independently selected from the group consisting of oxygen, sulfur, and nitrogen;
or X⁵ taken together with X^{5a} and the carbon atom to which they are attached form a bicyclic ring system consisting of a partially saturated or fully saturated 5- or 6-membered ring, optionally having 1 or 2 heteroatoms independently selected from the group consisting of nitrogen, sulfur, and oxygen, fused to a partially saturated, fully saturated, or fully unsaturated 5- or 6-membered ring, optionally having 1 to 4 heteroatoms independently selected from the group consisting of nitrogen, sulfur, and oxygen;
Z¹ is a bond, O, or N-X², provided that when a and b are both 0 then Z¹ is not N—X² or O;
R⁷ and R⁸ are each independently hydrogen or C₁-C₆ alkyl optionally independently substituted with A¹, —CO₂—(C₁-C₆ alkyl), —SOₘ(C₁-C₆ alkyl), 1 to 5 halo groups, 1 to 3 hydroxy groups, 1 to 3—O—CO(C₁-C₁₀ alkyl) groups, or 1 to 3 C₁-C₆ alkoxy groups; or
R⁷ and R⁸ can be taken together to form —(CH₂)ᵣ—L—(CH₂)r-, wherein L is CX²X², SOₘ, or NX²;
R⁹ and R¹⁰ are each independently selected from the group consisting of hydrogen, fluoro, hydroxy, and C₁-C₅ alkyl optionally independently substituted with 1-5 halo groups;
R¹¹ is selected from the group consisting of C₁-C₅ alkyl and phenyl optionally substituted with 1-3 substituents each independently selected from the group consisting of C₁-C₅ alkyl, halo, and C₁-C₅ alkoxy;
R¹² is selected from the group consisting of C₁-C₅ alkylsulfonyl, C₁-C₅ alkanoyl, and C₁-C₅ alkyl wherein the alkyl portion is optionally independently substituted by 1-5 halo groups;
A¹ for each occurrence is independently selected from the group consisting of C₅-C₇ cycloalkenyl, phenyl, a partially saturated, fully saturated, or fully unsaturated 4-to 8-membered ring optionally having 1 to 4 heteroatoms independently selected from the group consisting of oxygen, sulfur, and nitrogen, and a bicyclic ring system consisting of a partially saturated, fully unsaturated, or fully saturated 5- or 6-membered ring, optionally having 1 to 4 heteroatoms independently selected from the group consisting of nitrogen, sulfur, and oxygen, fused to a partially saturated, fully saturated, or fully unsaturated 5- or 6-membered ring, optionally having 1 to 4 heteroatoms independently selected from the group consisting of nitrogen, sulfur, and oxygen;
A¹ for each occurrence is independently optionally substituted, on one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —CONX⁶X⁶, —CO₂X⁶, oxo, C₁-C₆ alkyl, nitro, cyano, benzyl, —SOₘ(C₁-C₆ alkyl), 1H-tetrazol-5-yl, phenyl, phenoxy, phenylalkyloxy, halophenyl, methylenedioxy, -NX⁶X⁶,—NX⁶COX⁶, —SO₂NX⁶X⁶, —NX⁶SO₂-phenyl, NX⁶SO₂X⁶, —CONX¹¹X¹², SO₂ X¹¹X¹², —NX⁶SO₂X¹², —NX⁶CONX¹¹X², —NX⁶SO₂NX¹¹X¹², —NX⁶COX¹², imidazolyl, thiazolyl, and tetrazolyl, provided that if A¹ is optionally substituted with methylenedioxy then it can only be substituted with one methylenedioxy;
wherein X¹¹ is hydrogen or C₁-C₆ alkyl optionally independently substituted with phenyl, phenoxy, C₁-C₆ alkoxycarbonyl, —SOₘ(C₁-C₆ alkyl), 1 to 5 halo groups, 1 to 3 hydroxy groups, 1 to 3 C₁-C₁₀ alkanoyloxy groups, or 1 to 3 C₁-C₆ alkoxy groups;
X¹² is hydrogen, C₁-C₆ alkyl, phenyl, thiazolyl, imidazolyl, furyl, or thienyl, provided that when X¹² is not hydrogen, the X¹² group is optionally substituted with one to three substituents independently selected from the group consisting of Cl, F, CH₃, OCH₃, OCF₃, and CF₃;
or X¹¹ and X¹² are taken together to form —(CH₂)ᵣ—L¹—(CH₂)ᵣ—, wherein L¹ is CX²X², O, SOₘ, or NX²;
r for each occurrence is independently 1, 2, or 3;
X² for each occurrence is independently hydrogen, optionally substituted C₁-C₆ alkyl, or optionally substituted C₃-C₇ cycloalkyl, wherein the optionally substituted C₁-C₆ alkyl and optionally substituted C₃-C₇ cycloalkyl in the definition of X² are optionally independently substituted with —SOₘ(C₁-C₆ alkyl), —CO₂X³, 1 to 5 halo groups, or 1-3 OX³ groups;
X³ for each occurrence is independently hydrogen or C₁-C₆ alkyl;
X⁶ for each occurrence is independently hydrogen, optionally substituted C₁-C₆ alkyl, halogenated C₂-C₆ alkyl, optionally substituted C₃-C₇ cycloalkyl, halogenated C₃-C₇ cycloalkyl, wherein the optionally substituted C₁-C₆ alkyl and optionally substituted C₃-C₇ cycloalkyl in the definition of X⁶ are optionally independently mono- or di-substituted with C₁-C₄ alkyl, hydroxy, C₁-C₄ alkoxy, carboxyl, CONH₂, —SOₘ(C₁-C₆ alkyl), carboxylate (C₁-C₄ alkyl) ester, or 1H-tetrazol-5-yl; or
when there are two X⁶ groups on one atom and both X⁶ are independently C₁-C₆ alkyl, the two C₁-C₆ alkyl groups may be optionally joined, and together with the atom to which the two X⁶ groups are attached, form a 4- to 9- membered ring optionally having oxygen, sulfur, or NX⁷ as a ring member, wherein X⁷ is hydrogen or C₁-C₆ alkyl optionally substituted with hydroxy;
m for each occurrence is independently 0, 1, or 2; with the provisos that:
X⁶ and X¹² cannot be hydrogen when attached to CO or SO₂ in the form COX⁶, COX¹², SO₂X⁶ or SO₂X¹²; and
when R⁶ is a bond then L is NX² and each r in the definition —(CH₂)ᵣ—L—(CH₂)ᵣ—is independently 2 or 3.

18. A pharmaceutical composition according to claim 17 wherein said growth hormone secretagogue is
2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide;
2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide;
2-amino-N-{1(R)-benzyloxymethyl-2-[1,3-dioxo-8a(S)-pyridin-2-ylmethyl-2-(2,2,2-trifluoro-ethyl)-hexahydro-imidazo[1,5-a]pyrazin-7-yl]-2-oxo-ethyl}-2-methyl-propionamide;
N-(1 (R)-((1,2-dihydro-1-methanesulfonyl-spiro(3H-indole-3,4′-piperidin)-1′-yl)carbonyl)-2-(phenylmethyloxy)ethyl)-2-amino-2-methyl-propanamide; or
a prodrug of any of these compounds, or a pharmaceutically acceptable salt of any of these compounds or prodrugs.

19. A pharmaceutical composition according to claim 18 wherein said corticotropin releasing factor antagonist is 4-(1-ethyl-propoxy)-3,6-dimethyl-2-(2,4,6-trimethylphenoxy)-pyridine and said growth hormone secretagogue is 2-amino-N-[2-(3a(R)-benzyl-2-methyl-3-oxo-2,3,3a,4,6,7-hexahydro-pyrazolo-[4,3-c]pyridin-5-yl)-1(R)-benzyloxymethyl-2-oxo-ethyl]-isobutyramide.

20. A pharmaceutical composition according to claim 18 wherein said corticotropin releasing factor antagonist is 4-(1-ethyl-propoxy)-3,6-dimethyl-2-(2,4,6trimethylphenoxy)-pyridine and said growth hormone secretagogue is 2-amino-N-(1(R)-(2,4-difluoro-benzyloxymethyl)-2-oxo-2-(3-oxo-3a(R)-(pyridin-2-ylmethyl)-2-(2,2,2-trifluoro-ethyl)-2,3,3a,4,6 ,7-hexahydro-pyrazolo-[4,3-c]pyridin-5-yl)-ethyl)-2-methyl-propionamide.

21. A pharmaceutical composition according to claim 18 wherein said corticotropin releasing factor antagonist is (3,6-dimethyl-2-(2,4,6-trimethyl-phenoxy)-pyridin-4-yl)-(1-ethyl-propyl)-amine and said growth hormone secretagogue is 2-amino-N-[2-(3a(R)-benzyl-2-methyl-3-oxo-2,3,3a,4,6,7-hexahydro-pyrazolo-[4,3-c]pyridin-5-yl)-1(R)-benzyloxymethyl-2-oxo-ethyl]-isobutyramide.

22. A pharmaceutical composition according to claim 18 wherein said corticotropin releasing factor antagonist is (3,6-dimethyl-2-(2,4,6-trimethyl-phenoxy)-pyridin-4-yl)-(1-ethyl-propyl)-amine and said growth hormone secretagogue is 2-amino-N-(1(R)-(2,4-difluoro-benzyloxymethyl)-2-oxo-2-(3-oxo-3a(R)-(pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide.

23. A method for treating or preventing osteoporosis or frailty associated with aging or obesity, said method comprising administering to a human or other animal an amount of a pharmaceutical composition according to claim 1, which is effective in treating or preventing osteoporosis or frailty associated with aging or obesity.

24. A method for treating or preventing a cardiovascular or heart related disease, said method comprising administering to a human or other animal an amount of a pharmaceutical composition according to claim 1, which is effective in treating or preventing the cardiovascular or heart related disease.

25. A method according to claim 24 wherein the cardiovascular or heart related disease is hypertension, tachycardia, or congestive heart failure.

26. A method for accelerating bone fracture repair, attenuating protein catabolic response after a major operation, reducing cachexia and protein loss due to chronic illness, accelerating wound healing, or accelerating the recovery of burn patients or of patients having undergone major surgery, said method comprising administering to a human or other animal an amount of a pharmaceutical composition according to claim 1, which is effective in accelerating bone fracture repair, attenuating protein catabolic response after a major operation, reducing cachexia and protein loss due to chronic illness, accelerating wound healing, or accelerating the recovery of burn patients or of patients having undergone major surgery.

27. A method for treating or preventing osteoporosis, frailty associated with aging or obesity, cardiovascular or heart related disease, for accelerating bone fracture repair, attenuating protein catabolic response after a major operation, reducing cachexia and protein loss due to chronic illness, accelerating wound healing, or accelerating the recovery of burn patients or of patients having undergone major surgery, said method comprising administering to a human or other animal an amount of a corticotropin releasing factor antagonist and an amount of a growth hormone secretagogue or growth hormone.

28. The method of claim 27 wherein said corticotropin releasing factor antagonist and said growth hormone secretagogue or growth hormone are administered simultaneously or in a specifically timed manner.

29. A kit comprising:
a. an amount of a corticotropin releasing factor antagonist, in a first unit dosage form;
b. an amount of a growth hormone secretagogue or growth hormone, in a second unit dosage form; and
c. a container.

30. A kit comprising:
a. an amount of a corticotropin releasing factor antagonist as defined in claim 13, in a first unit dosage form;
b. an amount of a growth hormone secretagogue or growth hormone, in a second unit dosage form; and
c. a container.

31. A kit comprising:
a. an amount of a corticotropin releasing factor antagonist as defined in claim 14, in a first unit dosage form;
b. an amount of a growth hormone secretagogue or growth hormone, in a second unit dosage form; and
c. a container.

32. A kit comprising:
a. an amount of a corticotropin releasing factor antagonist, in a first unit dosage form;
b. an amount of a growth hormone secretagogue as defined in claim 15, in a second unit dosage form; and
c. a container.

33. A kit according to claim 29 wherein said corticotropin releasing factor antagonist is 4-(1-ethyl-propoxy)-3,6-dimethyl-2-(2,4,6-trimethylphenoxy)-pyridine or [3,6-dimethyl-2-(2,4,6-trimethyl-phenoxy)-pyridin-4-yl]-(1-ethyl-propyl)-amine, and said growth hormone secretagogue is 2-amino-N-[2-(3a(R)-benzyl-2-methyl-3-oxo-2,3,3a,4,6,7-hexahydro-pyrazolo-[4,3-c]pyridin-5-yl)-1(R)-benzyloxymethyl-2-oxo-ethyl]-isobutyramide or 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide.

34. A kit, comprising
a. a pharmaceutical composition, comprising an amount of a growth hormone or growth hormone secretagogue;
b. a package containing the above composition; and
c. a package insert that may be integral with said package;
wherein it is stated on the package insert that the pharmaceutical composition is to be administered simultaneously or in a specifically timed manner with a pharmaceutical composition containing at least one corticotropin releasing factor antagonist.

35. A kit, comprising
a. a pharmaceutical composition, comprising an amount of a corticotropin releasing factor antagonist;
b. a package containing the above composition; and
c. a package insert that may be integral with said package;
wherein it is stated on the package insert that the pharmaceutical composition is to be administered simultaneously or in a specifically timed manner with a pharmaceutical composition containing at least one growth hormone or growth hormone secretagogue.
